(19) United States
(12) Patent Application Publication
(10) Pub. No.: US 2025/0186354 A1
(43) Pub. Date: Jun. 12, 2025

(54) Steroidal Testosterone Ester Compositions and Preparation Thereof

(71) Applicant: Lipocine Inc., Salt Lake City, UT (US)

(72) Inventors: Joel Frank, Salt Lake City, UT (US); Samuel Akapo, Salt Lake City, UT (US); Mahesh V. Patel, Salt Lake City, UT (US); Nachiappan Chidambaram, Salt Lake City, UT (US)

(73) Assignee: Lipocine Inc., Salt Lake City, UT (US)

(21) Appl. No.: 18/598,151

(22) Filed: Mar. 7, 2024

Related U.S. Application Data
(60) Provisional application No. 63/489,811, filed on Mar. 13, 2023.

(51) Int. Cl. A61K 9/48; A61K 31/568

(52) U.S. Cl. CPC A61K 9/4833; A61K 9/4858; A61K 9/4866; A61K 31/568

(57) ABSTRACT

Provided herein are steroid containing compositions suitable for providing therapeutically effective amounts of at least one steroid to individuals. Also provided herein are compositions comprising testosterone and/or testosterone derivatives suitable for providing therapeutically effective and safe amounts of testosterone over periods of time. Further provided are methods of treating andro- and/or testosterone deficiency in individuals by administering to the individuals compositions described herein. Further provided are methods of preparing steroidal testosterone ester containing compositions and products.

### CROSS REFERENCE TO RELATED APPLICATIONS

This nonprovisional utility patent application claims the benefit under 35 USC § 119(e) of U.S. provisional application No. 63/489,811 filed Mar. 13, 2023, which is expressly incorporated herein in its entirety by this reference. This application is also related to U.S. Pat. No. 11,052,096 issued Jul. 6, 2021, which is incorporated herein in its entirety by reference.

### FIELD OF THE INVENTION

The present invention relates to testosterone undecanoate containing pharmaceutical compositions and oral dosage units as well as associated methods of preparation and use. Accordingly, this invention involves the fields of chemistry, pharmaceutical sciences, medicine and other health sciences.

### BACKGROUND OF THE INVENTION

Testosterone is an androgenic compound crucial for human health. Certain embodiments of the invention described herein generally relate to compositions for the administration of testosterone, testosterone analogs, other steroids and related compounds.

### BRIEF SUMMARY OF THE INVENTION

Provided in certain embodiments herein is a delayed release oral dosage form comprising a therapeutically effective amount of one or more testosterone alkyl ester and at least one pharmaceutically acceptable carrier, wherein a single dose of the delayed release oral dosage form provides a mean plasma Cₘₐₓ of testosterone that is at least 5%, at least 10% or at least 15% lower than the mean plasma Cₘₐₓ of testosterone that is provided by a single dose of an immediate release oral dosage form having an identical amount of the testosterone alkyl ester. In some embodiments, provided herein is a delayed release oral dosage form, wherein a single dose of the delayed release oral dosage form provides a mean plasma Cₘₐₓ of testosterone alkyl ester that is at least 5%, at least 10% or at least 15% lower than the mean plasma Cₘₐₓ of testosterone alkyl ester that is provided by a single dose of an immediate release oral dosage form having an identical amount of the testosterone alkyl ester. In certain embodiments, a single dose of the delayed release oral dosage form described herein provides a mean plasma Cₘₐₓ of that is at least 5% lower than the mean plasma Cₘₐₓ of dihydrotestosterone provided by a single dose of an immediate release oral dosage form having an identical amount of the testosterone alkyl ester. In some embodiments, a delayed release oral dosage form described herein provides a mean plasma Cₘₐₓ at steady state of testosterone alkyl ester that is at least 5%, at least 10% or at least 15% lower than the mean plasma Cₘₐₓ of testosterone alkyl ester at steady state provided by an immediate release oral dosage form having an identical amount of the testosterone alkyl ester. In certain embodiments, a delayed release oral dosage form described herein provides a fluctuation index of testosterone at steady state that is at least 5%, or at least 10% lower than a fluctuation index of testosterone at steady state of an immediate release oral dosage form having an identical amount of the testosterone alkyl ester. In some embodiments, a delayed release oral dosage form described herein provides a fluctuation index of testosterone alkyl ester at steady state that is at least 5%, or at least 10% lower than a fluctuation index of testosterone alkyl ester at steady state of an immediate release oral dosage form having an identical amount of the testosterone alkyl ester. In some embodiments, a single dose of the delayed release oral dosage form provides a mean plasma concentration of testosterone provided 1 hour after oral administration of the delayed release oral dosage form that is at least 20% lower than a mean plasma concentration of testosterone provided 1 hour after oral administration of a single dose of an immediate release oral dosage form having an identical amount of the testosterone alkyl ester.

Provided in certain embodiments herein is a pharmaceutical composition comprising a therapeutically effective amount of one or more testosterone alkyl ester and at least one pharmaceutically acceptable carrier. In some embodiments, a single dose of a pharmaceutical composition described herein provides a mean plasma Cₘₐₓ of testosterone that is about 15 ng/mL or less; or about 19 ng/mL or less upon oral administration. In certain embodiments, a single dose of a pharmaceutical composition described herein provides a mean plasma Cₘₐₓ of dihydrotestosterone that is about 4.5 ng/mL, or about 3.6 ng/mL or less upon oral administration. In some embodiments, a pharmaceutical composition described herein provides a testosterone mean plasma Cₘₐₓ at steady state of about 1300 ng/dL or less. In certain embodiments, a pharmaceutical composition described herein provides a testosterone mean plasma Cₘᵢₙ at steady state of about 200 ng/dL or more. In some embodiments a pharmaceutical composition provides with administration to an individual a ratio of the testosterone equivalent dose from the alkyl ester, to a mean steady state testosterone Cₘₐₓ, the ratio being about 500×10⁶ mL or less. In certain embodiments, the difference between the mean plasma Cₘₐₓ of testosterone at steady state and mean plasma Cₘᵢₙ of testosterone at steady state provided by a pharmaceutical composition described herein is about 11 ng/mL or less, or about 16 ng/mL or less. In some embodiments, the difference between the mean plasma Cₘₐₓ at steady state and mean plasma Cₘᵢₙ at steady state of testosterone alkyl ester provided by a pharmaceutical composition described herein is about 275 ng/mL or less; or about 200 ng/mL or less. In certain embodiments, a single dose of a pharmaceutical composition described herein provides a mean plasma concentration of testosterone after 1 hour that is about 150 ng/dL or less upon oral administration. In some embodiments, a single dose of a pharmaceutical composition described herein provides a mean plasma concentration of testosterone after 2 hours that is about 500 ng/dL or less upon oral administration.

In certain embodiments, a pharmaceutical composition described herein releases about 50% or less of the testosterone alkyl ester after 1 hour and/or about 80% or less of the testosterone alkyl ester after about 30 minutes in an aqueous medium. In some embodiments, a pharmaceutical composition described herein releases about 20% or less of the testosterone alkyl ester after 30 minutes in an aqueous medium. In certain embodiments, a pharmaceutical composition described herein releases less than 95% of the testosterone alkyl ester after 3 hours in an aqueous medium. In some embodiments, a pharmaceutical composition described herein releases more than 80% of the testosterone alkyl ester within 12 hours in an aqueous medium. In some instances, the aqueous medium is present in a USP Type-II (paddle) apparatus with conditions at 37±0.5° C. and at 100 rpm. In more specific instances, the aqueous medium is about 1 L of DI water having 8% w/v of Triton X-100.

In certain embodiments, described herein is a delayed release oral dosage form comprising a testosterone alkyl ester (e.g., testosterone alkyl ester formulated in solid PEG). In some embodiments, a pharmaceutical composition described herein is a delayed release oral dosage form. In certain embodiments, the delayed release oral dosage form is formulated in any suitable manner. In some embodiments, a single dose of a delayed release oral dosage form described herein provides a mean plasma Cₘₐₓ of testosterone that is at least about 5%, at least 10% or at least 15% lower than the mean plasma Cₘₐₓ of testosterone that is provided by a single dose of an immediate release oral dosage form having an identical amount of the testosterone alkyl ester. In certain embodiments, a single dose of a delayed release oral dosage form described herein provides a mean plasma Cₘₐₓ of the testosterone alkyl ester that is at least about 5%, at least 10% or at least 15% lower than the mean plasma Cₘₐₓ of testosterone alkyl ester that is provided by a single dose of an immediate release oral dosage form having an identical amount of the testosterone alkyl ester. In some embodiments, a single dose of a delayed release oral dosage form described herein provides a mean plasma Cₘₐₓ of that is at least 5% lower than the mean plasma Cₘₐₓ of dihydrotestosterone provided by a single dose of an immediate release oral dosage form having an identical amount of the testosterone alkyl ester. In certain embodiments, a delayed release oral dosage form described herein provides a mean plasma Cₘₐₓ at steady state of testosterone alkyl ester that is at least about 5%, at least 10% or at least 15% lower than the mean plasma Cₘₐₓ of testosterone alkyl ester at steady state provided by an immediate release oral dosage form having an identical amount of the testosterone alkyl ester. In some embodiments, a delayed release oral dosage form described herein provides a fluctuation index of testosterone at steady state that is at least 10% lower than a fluctuation index of testosterone at steady state of an immediate release oral dosage form having an identical amount of the testosterone alkyl ester. In certain embodiments, a delayed release oral dosage form described herein provides a fluctuation index of testosterone alkyl ester at steady state that is at least 10% lower than a fluctuation index of testosterone alkyl ester at steady state of an immediate release oral dosage form having an identical amount of the testosterone alkyl ester. In some embodiments, a single dose of a delayed release oral dosage form described herein provides a mean plasma concentration of testosterone provided 1 hour after oral administration of the delayed release oral dosage form that is at least 20% lower than a mean plasma concentration of testosterone provided 1 hour after oral administration of a single dose of an immediate release oral dosage form having an identical amount of the testosterone alkyl ester.

In some embodiments, the one or more testosterone alkyl ester provided in any pharmaceutical composition or oral dosage form described herein is or comprises testosterone undecanoate (TU). In certain embodiments, any pharmaceutical composition or oral dosage form described herein comprises about 10 mg to about 400 mg, or about 10 mg to about 1000 mg of testosterone alkyl ester. In some embodiments, any pharmaceutical composition or oral dosage form described herein comprises about 10 mg to about 300 mg of testosterone alkyl ester. In certain embodiments, any pharmaceutical composition or oral dosage form described herein comprises about 10 mg to about 240 mg of testosterone alkyl ester. In some embodiments, any pharmaceutical composition or oral dosage form described herein comprises about 10 mg to about 150 mg of testosterone alkyl ester. In some embodiments, any pharmaceutical composition or oral dosage form described herein comprises about 120 mg of testosterone alkyl ester.

In certain embodiments, the at least one pharmaceutically acceptable carrier of any pharmaceutical composition or oral dosage form described herein comprises at least one hydrophilic carrier. In some embodiments, the at least one pharmaceutically acceptable carrier of any pharmaceutical composition or oral dosage form described herein comprises at least one lipophilic carrier. In certain embodiments, the at least one pharmaceutically acceptable carrier of any pharmaceutical composition or oral dosage form described herein comprises at least one viscosity enhancer or solidifying agent. In some embodiments, the at least one hydrophilic carrier comprises a hydrophilic triglyceride. In specific embodiments, the hydrophilic triglyceride is a polyoxylated castor oil, or a polyoxylated hydrogenated castor oil.

Provided in some embodiments herein is a method of treating androgen deficiency in an individual in need thereof by administering to the individual any oral dosage form or pharmaceutical composition described herein. In some embodiments, a pharmaceutical composition or oral dosage form described herein is administered b.i.d. In certain embodiments, a pharmaceutical composition or oral dosage form described herein is administered with a meal.

Provided in certain embodiments herein is an oral testosterone undecanoate therapy that provides to a human in need of androgen therapy by orally delivering to the human a composition comprising a therapeutically effective amount of testosterone undecanoate. In some embodiments, the oral testosterone undecanoate therapy provides in a human (e.g., a male human) a mean Cₘₐₓ of testosterone that is less than about 15 ng/mL; or less than about 19 ng/mL after a single administration of the composition. In certain embodiments, the oral testosterone undecanoate therapy provides to a human (e.g., a male human) a mean plasma Cₘₐₓ of dihydrotestosterone that is about 3.6 ng/mL or less; or about 4.5 ng/mL or less after a single administration of the composition. In some embodiments, the oral testosterone undecanoate therapy provides to a human (e.g., a male human) a testosterone mean plasma Cₘₐₓ at steady state of about 1300 ng/dL or less. In certain embodiments, the oral testosterone undecanoate therapy provides to a human (e.g., a male human) a testosterone mean plasma Cₘᵢₙ at steady state of about 200 ng/dL or more. In some embodiments, the oral testosterone undecanoate therapy provides to a human (e.g., a male human) a mean Cₘₐₓ of testosterone at steady state to dose ratio of about 15 or less. In specific embodiments, the ratio is 15 or less, or 13 or less. In some embodiments provided herein is a pharmaceutical composition that provides with administration to an individual a ratio of a testosterone C₂-C₁₃ alkyl ester dose, in mg, to a mean steady state testosterone Cₘₐₓ, in mg/mL, the ratio of testosterone equivalent dose from the testosterone alkyl ester to a mean steady state testosterone Cₘₐₓ, the ratio being about 500×10⁶ mL or less (e.g., with b.i.d. or q.d. administration to an otherwise testosterone deficient individual). In certain embodiments, the oral testosterone undecanoate therapy provides to a human (e.g., a male human) a difference between a mean plasma Cₘₐₓ of testosterone at steady state and mean plasma Cₘᵢₙ of testosterone at steady state of about 11 ng/mL or less, or about 16 ng/mL or less. In some embodiments, the oral testosterone undecanoate therapy provides to a human (e.g., a male human) a difference between a mean plasma Cₘₐₓ at steady state and mean plasma Cₘᵢₙ at steady state of testosterone alkyl ester of about 200 ng/mL or less; or about 275 ng/mL or less. In certain instances, when a mean plasma concentration is utilized, the value is obtained from a statistically significant population of individuals.

Provided in certain embodiments herein is a pharmaceutical composition comprising (i) a therapeutically effective amount of one or more testosterone C₂-C₁₃ alkyl ester; and (ii) at least one pharmaceutically acceptable carrier; the pharmaceutical composition releasing about 80% or less of the testosterone C₂-C₁₃ alkyl ester after 30 minutes in an aqueous medium. In certain instances, the aqueous medium comprises 8% w/v octoxynol-9 in water at about 37° C. In some embodiments, any aqueous medium described herein is 1 L deionized water comprising 8% w/v Triton X-100 (e.g., octylphenol ethylene oxide condensate; octoxynol-9; t-octylphenoxypolyethoxyethanol; t-oct-C₆H₄—(OCH₂CH₂)ₓOH, x=9-10; CAS No. 9002-93-1; Triton X-100 was a registered trademark formerly owned by Rohm and Haas Co., but now owned by Union Carbide) at 37±0.5° C. and subjected to a paddle method at 100 rpm and 37±0.5° C. for the designated period of time (USP App 2). In some embodiments, the testosterone C₂-C₁₃ alkyl ester is testosterone undecanoate. In certain embodiments, the pharmaceutical composition comprises about 10 mg to about 1000 mg of testosterone C₂-C₁₃ alkyl ester.

In some embodiments, a single dose of any pharmaceutical composition provided herein provides a mean plasma Cₘₐₓ of testosterone that is about 15 ng/mL or less; or about 19 ng/mL or less upon oral administration (e.g., to a testosterone deficient individual). In certain embodiments, a single dose of any pharmaceutical composition provided herein provides a mean plasma Cₘₐₓ of dihydrotestosterone that is about 4.5 ng/mL or less; or about 3.6 ng/mL or less upon oral administration (e.g., to a testosterone deficient individual). In some embodiments, any pharmaceutical composition provided herein provides a testosterone mean plasma Cₘₐₓ at steady state of about 1300 ng/dL or less with oral administration (e.g., with b.i.d. or q.d. administration to an otherwise testosterone deficient individual). In certain embodiments, any pharmaceutical composition provided herein provides a testosterone mean plasma Cₘᵢₙ at steady state of about 200 ng/dL or more with oral administration (e.g., with b.i.d. or q.d. administration to an otherwise testosterone deficient individual). In some embodiments, any pharmaceutical composition provided herein provides with administration to an individual (e.g., oral administration) a ratio of testosterone equivalent dose from the testosterone alkyl ester to a mean a mean steady state testosterone Cₘₐₓ, the ratio being about 500×10⁶ mL, or less (e.g., with b.i.d. or q.d. administration to an otherwise testosterone deficient individual).

In some embodiments, the difference between the mean plasma Cₘₐₓ of testosterone at steady state and mean plasma Cₘᵢₙ of testosterone at steady state is about 11 ng/mL or less, or about 16 ng/mL or less (e.g., with b.i.d. or q.d. administration to an otherwise testosterone deficient individual). In some embodiments, the difference between the mean plasma Cₘₐₓ at steady state and mean plasma Cₘᵢₙ at steady state of testosterone C₂-C₁₃ alkyl ester is about 200 ng/mL or less; or about 275 ng/mL or less (e.g., with b.i.d. or q.d. administration to an otherwise testosterone deficient individual). In some embodiments, a single dose of any pharmaceutical composition provided herein provides a mean plasma concentration of testosterone after 1 hour that is about 150 ng/dL or less upon oral administration. In certain embodiments, a single dose of any pharmaceutical composition provided herein provides a mean plasma concentration of testosterone after 2 hours that is about 500 ng/dL or less upon oral administration.

In certain embodiments, the at least one pharmaceutically acceptable carrier of any pharmaceutical composition provided herein comprises at least one hydrophilic carrier. In specific embodiments, the hydrophilic carrier is a hydrophilic triglyceride. In more specific embodiments, the hydrophilic triglyceride is a polyoxylated castor oil, or a polyoxylated hydrogenated castor oil. In some embodiments, any pharmaceutical composition provided herein consists essentially of a lipophilic carrier or combination of lipophilic carriers. In certain embodiments, any pharmaceutical composition provided herein comprises a lipophilic carrier and less than 10% w/w or less than 5% w/w of a hydrophilic carrier.

Provided in certain embodiments herein is a delayed release oral dosage form comprising (i) a therapeutically effective amount of one or more testosterone C₂-C₁₃ alkyl ester; and (ii) at least one pharmaceutically acceptable carrier; wherein a single dose of the delayed release oral dosage form provides a mean plasma Cₘₐₓ of testosterone that is at least 5% lower; or at least 10% lower than the mean plasma Cₘₐₓ of testosterone that is provided by a single dose of an immediate release oral dosage form having an identical amount of the testosterone C₂-C₁₃ alkyl ester. In some embodiments, the testosterone C₂-C₁₃ alkyl ester is testosterone undecanoate. In certain embodiments, the pharmaceutical composition comprises about 10 mg to about 1000 mg of testosterone C₂-C₁₃ alkyl ester.

In some embodiments, a single dose of any delayed release oral dosage form provided herein provides a mean plasma Cₘₐₓ of the that is at least 5%, at least 10% or at least 15% lower than the mean plasma Cₘₐₓ of testosterone C₂-C₁₃ alkyl ester that is provided by a single dose of an immediate release oral dosage form having an identical amount of the testosterone C₂-C₁₃ alkyl ester.

In some embodiments, a single administration to a human of a dose of the delayed release oral dosage form provides a ratio of testosterone equivalent dose from the C₂-C₁₃ alkyl ester present in the dose of the delayed release oral dosage form to mean plasma testosterone Cₘₐₓ provided by the single administration of the dose of the delayed oral release dosage form, the ratio being about 500×10⁶ mL or less. In certain embodiments, a single dose of any delayed release oral dosage form provided herein provides a mean plasma Cₘₐₓ of that is at least 5% lower than the mean plasma Cₘₐₓ of dihydrotestosterone provided by a single dose of an immediate release oral dosage form having an identical amount of the testosterone C₂-C₁₃ alkyl ester. In some embodiments, a single administration to a human a dose of the delayed release oral dosage form provides a ratio of testosterone equivalent dose from the C₂-C₁₃ alkyl ester to mean plasma dihydroxytestosterone Cₘₐₓ provided by the single administration of the dose of the delayed oral release dosage form, the ratio being about 350×10⁶ mL or less. In some embodiments, any delayed release oral dosage form provided herein provides a mean plasma Cₘₐₓ at steady state of testosterone C₂-C₁₃ alkyl ester that is at least 5% lower, or at least 10% lower than the mean plasma Cₘₐₓ of testosterone C₂-C₁₃ alkyl ester at steady state provided by an immediate release oral dosage form having an identical amount of the testosterone C₂-C₁₃ alkyl ester (e.g., when orally administered to a testosterone deficient individual b.i.d. or q.d.).

In certain embodiments, any delayed release oral dosage form provided herein comprises at least one pharmaceutically acceptable carrier that comprises at least one hydrophilic carrier. In specific embodiments, the hydrophilic carrier is a hydrophilic triglyceride. In more specific embodiments, the hydrophilic triglyceride is a polyoxylated castor oil, or a polyoxylated hydrogenated castor oil. In some embodiments, any delayed release oral dosage form provided herein consists essentially of a lipophilic carrier or combination of lipophilic carriers. In some embodiments, a lipophilic carrier selected from the group consisting of a monoglyceride, a diglyceride, a Vitamin E compound, a triglyceride, a fatty acid, polyoxylated fatty acid, polyoxylated triglyceride, polyoxylated vegetable oil, and a combination thereof. In certain embodiments, any delayed release oral dosage form provided herein comprises a lipophilic carrier and less than 10% w/w or less than 5% w/w of a hydrophilic carrier.

Provided in some embodiments herein is a pharmaceutical composition comprising (i) a therapeutically effective amount of one or more testosterone alkyl ester; and (ii) at least one pharmaceutically acceptable carrier; the pharmaceutical composition releasing about 60% to about 90%, about 60% to about 85%, or about 60% to about 80% of the testosterone alkyl ester after 1 hour in an aqueous medium. In certain instances, the aqueous medium comprises 8% w/v octoxynol-9 in water at about 37° C.

Provided in certain embodiments herein is a pharmaceutical composition comprising (i) a therapeutically effective amount of one or more testosterone alkyl ester; and (ii) at least one pharmaceutically acceptable carrier; the pharmaceutical composition releasing about 50% or less, about 45% or less, or about 40% or less of the testosterone alkyl ester after 6 hour in an aqueous medium. In certain instances, the aqueous medium comprises 8% w/v octoxynol-9 in water at about 37° C.

Provided in some embodiments herein is a method of treating androgen deficiency in an individual in need thereof by administering to the individual any pharmaceutical composition or dosage form described herein. In a specific embodiment, provided herein is a method of treating androgen deficiency in an individual in need thereof by administering to the individual a pharmaceutical composition comprising (i) a therapeutically effective amount of one or more testosterone C₂-C₁₃ alkyl ester; and (ii) at least one pharmaceutically acceptable carrier. In specific embodiments, the pharmaceutical composition releases about 80% or less; or 90% or less of the testosterone C₂-C₁₃ alkyl ester after 30 minutes in an aqueous medium. In some embodiments, the pharmaceutical composition releases about 50% or less of the testosterone C₂-C₁₃ alkyl ester after 1 hour in an aqueous medium. In certain embodiments, the pharmaceutical composition is administered with a meal. In some embodiments, the pharmaceutical composition is administered b.i.d. or q.d. In various embodiments, a method provided herein has a release or pharmacokinetic profile as described herein. In some embodiments, an oral testosterone undecanoate therapy described herein provides to a human a ratio of a testosterone equivalent dose from the testosterone C₂-C₁₃ alkyl ester to mean steady state testosterone Cₘₐₓ, the ratio being about 500×10⁶ mL or less.

Also provided in some embodiments herein is an oral testosterone undecanoate therapy that provides to a human in need of androgen therapy by orally delivering to the human a composition comprising a therapeutically effective amount of testosterone undecanoate. In some embodiments, the therapy provides to the human a mean Cₘₐₓ of testosterone that is less than about 15 ng/mL, or less than about 19 ng/mL after a single administration of the composition. In certain embodiments, the oral testosterone undecanoate therapy provides to the human a mean plasma Cₘₐₓ of dihydrotestosterone that is about 3.6 ng/mL, or less; or about 4.5 ng/mL, or less after a single administration of the composition. In some embodiments, the oral testosterone undecanoate therapy provides to the human a testosterone mean plasma Cₘₐₓ at steady state of about 1300 ng/dL or less after a single administration of the composition. In certain embodiments, the oral testosterone undecanoate therapy provides to the human a testosterone mean plasma Cₘᵢₙ at steady state of about 200 ng/dL or more after a single administration of the composition. In some embodiments, the oral testosterone undecanoate therapy provides to the human a ratio of a testosterone equivalent dose to a mean stead state testosterone Cₘₐₓ of about 500×10⁶ mL or less after a single administration of the composition. In certain embodiments, the oral testosterone undecanoate therapy provides to the human a difference between a mean plasma Cₘₐₓ of testosterone at steady state and mean plasma Cₘᵢₙ of testosterone at steady state of about 11 ng/mL or less; or about 16 ng/mL or less. In some embodiments, the oral testosterone undecanoate therapy provides to the human a difference between a mean plasma Cₘₐₓ at steady state and mean plasma Cₘᵢₙ at steady state of testosterone alkyl ester of about 200 ng/mL or less.

Provided in certain embodiments herein is a pharmaceutical composition comprising (i) a therapeutically effective amount of one or more testosterone alkyl ester; and (ii) a single (e.g., one and only one) lipid component solubilizing the testosterone alkyl ester. In some embodiments, provided herein is a pharmaceutical composition comprising a therapeutically effective amount of testosterone undecanoate; the pharmaceutical composition providing an increase in testosterone alkyl ester in plasma compared to an otherwise identical pharmaceutical composition comprising a testosterone alkyl ester other than testosterone undecanoate. In certain embodiments, provided herein is a pharmaceutical composition comprising (i) a therapeutically effective amount of one or more testosterone alkyl ester; and (ii) at least one pharmaceutically acceptable carrier; the pharmaceutical composition providing, when administered as a single dose to an individual, a dose of testosterone equivalent from the testosterone alkyl ester, to mean steady state AUC_{0-∞} ratio of about 500×10³ mL/h or less.

### BRIEF DESCRIPTION OF THE FIGURES

The novel features of the invention are set forth with particularity in the appended claims. A better understanding of the features and advantages of the present invention may be obtained by reference to the following detailed description that sets forth illustrative embodiments, in which the principles of the invention, in certain embodiments, are utilized, and the accompanying drawings of which:

FIG. 1 illustrates the release profiles of Capsules 1-4 subjected to USP Apparatus 2 at 37° C. and 100 rpm;

FIG. 2 illustrates the mean plasma testosterone concentrations following administration of several oral dosage forms described herein and an immediate release oral dosage;

FIG. 3 illustrates the mean plasma testosterone undecanoate concentrations following administration of several oral dosage forms described herein and an immediate release oral dosage;

FIG. 4 illustrates the mean plasma dihydrotestosterone concentrations following administration of several oral dosage forms described herein and an immediate release oral dosage; and

FIG. 5 illustrates first and second release profiles (curves) plotted on a graph having dissolution amount on a first axis and time on a second axis.

### DETAILED DESCRIPTION

Provided herein are pharmaceutical compositions and methods of using the same. In some embodiments, the pharmaceutical compositions are formulated for oral delivery as an oral dosage form. In certain embodiments, a pharmaceutical composition described herein comprises a steroidal compound and at least one pharmaceutically acceptable carrier. In some embodiments, a pharmaceutical composition described herein is an oral dosage form comprising a steroidal compound and at least one pharmaceutically acceptable carrier. In specific embodiments, the steroidal compound is a steroidal androgen (e.g., testosterone, dihydrotestosterone, analogs, or prodrugs thereof). In certain embodiments, analogs or prodrugs of testosterone include, e.g., esters of testosterone. In specific embodiments, the esters of testosterone include, e.g., alkyl (e.g., straight chain, branched, cyclic, unsaturated, partially saturated, fully saturated and the like) esters of testosterone. Specifically, alkyl esters of testosterone include, by way of non-limiting example, lower alkyl esters (e.g., testosterone C₂-C₁₃ alkyl esters such as testosterone propionate, testosterone enanthate, or testosterone undecanoate), or higher alkyl esters (e.g., testosterone C₁₄₊ alkyl esters such as testosterone palmitate).

In further embodiments, the alkyl esters of testosterone include, by way of non-limiting example, cycloalkylalkyl esters (e.g., testosterone cypionate), cycloalkyl esters, and alkylcycloalkyl esters. In more specific embodiments, the testosterone alkyl ester is testosterone undecanoate. In certain embodiments, alkyl groups of the alkyl esters and/or other positions of the steroidal compound (e.g., testosterone alkyl ester, such as testosterone undecanoate) described herein are optionally substituted, e.g., with one or more halogen, hydroxy group, amino group, or the like, or combinations thereof.

In various embodiments, the pharmaceutical compositions are formulated for androgen (e.g., testosterone) therapy. In certain instances, the androgen therapy is an androgen (e.g., testosterone) replacement therapy. In some embodiments, the androgen replacement therapy is utilized to treat individuals suffering from androgen deficiency (e.g., postmenopausal women, menopausal women, sexually dysfunctional women, andropausal men, hypogonadal men, and the like) or treat individuals in need of increased androgen levels. In some embodiments, the androgen (e.g., testosterone) replacement therapy is utilized for the treatment of individuals diagnosed with or exhibiting symptoms of androgen (e.g., testosterone) deficiency including, e.g., in aging men.

Provided in certain embodiments herein are pharmaceutical compositions that provide a plasma Cₘₐₓ of testosterone that is less than 1500 ng/dL in at least 85% of a population of individuals (following administration of a single dose and/or in the steady state) when administered to a population of individuals (e.g., adult and/or pubescent human males). In some embodiments a pharmaceutical composition described herein provides a plasma Cₘₐₓ of testosterone that is less than 1800 ng/dL in at least 95% of a population of individuals (following administration of a single dose and/or in the steady state) when administered to a population of individuals (e.g., adult and/or pubescent human males). In some embodiments the oral dosage forms provide a plasma Cₘₐₓ of testosterone that is less than 2500 ng/dL in all individuals (following administration of a single dose and/or in the steady state) when administered to a population of individuals (e.g., adult and/or pubescent human males). In some embodiments, the individuals are adult humans. In specific embodiments, the adult humans are adult hypogonadal or otherwise androdeficient male humans.

In certain instances, normal human male testes produce four to eight milligrams of testosterone daily and human females produce less. Within certain contexts of the invention described herein, it will be generally recognized by those of skill in the art that the physiological “normal” range of total testosterone in men is about 250 to about 1,100 nanograms per deciliter (ng/dL) and in healthy women is about 11 ng/dL to about 78 ng/dL. Journal of Clinical Endocrinology & Metabolism, 85(7):2395-401.

Provided in some embodiments herein are pharmaceutical compositions that provide a Cₘᵢₙ that is about 10 ng/dL or greater and a Cₘₐₓ that is about 100 ng/dL or less in at least 50%, at least 60%, at least 70%, at least 80%, at least 90%, at least 95% of adult female humans (e.g., postmenopausal or otherwise androdeficient female humans) when administered to a population of adult female humans (following administration of a single dose and/or in the steady state). Provided in some embodiments herein are pharmaceutical compositions that provide a Cₘᵢₙ that is about 12 ng/dL or greater and a Cₘₐₓ that is about 82 ng/dL or less in at least 50%, at least 60%, at least 70%, at least 80%, at least 90%, at least 95% of adult female humans (e.g., postmenopausal or otherwise androdeficient female humans) when administered to a population of adult female humans (following administration of a single dose and/or in the steady state).

In some embodiments, the pharmaceutical composition provided herein is a delayed release oral dosage form comprising a steroidal compound and at least one pharmaceutically acceptable carrier. In certain instances, the delayed release oral dosage forms release the active in an aqueous medium (e.g., water, gastric fluid, or an aqueous solution with a pH of about 5.8) at a rate slower than an immediate or fast release oral dosage form (e.g., as measured by the amount of active found in the aqueous medium). In some embodiments, delayed release oral dosage forms comprise a steroidal compound, at least one hydrophilic carrier. In further embodiments, delayed release oral dosage forms comprise a steroidal compound, at least one hydrophilic carrier, and at least one lipidic and/or lipophilic carrier. In still further embodiments, the delayed release oral dosage form comprises at least one steroidal compound, at least one hydrophilic carrier, at least one lipidic and/or lipophilic carrier, and at least one viscosity enhancer or solidifying agent. In still further embodiments, the delayed release oral dosage form comprises at least one steroidal compound and at least one viscosity enhancer or solidifying agent. In some embodiments, a pharmaceutical composition provided herein is formulated, e.g., with the viscosity enhancing agent or solidifying agent, to provide a solid, a semi-solid, a gel, a jelly, a paste, or the like. In specific embodiments, the delayed release oral dosage form is a capsule (e.g., a hard- or soft-gel capsule, a tablet or other solid dosage form). In some embodiments, the delayed release dosage form provided herein comprises the active (e.g., one or more testosterone alkyl ester such as testosterone undecanoate) in different release fractions (e.g., an immediate release portion and a delayed release portion). In specific embodiments, pharmaceutical compositions or dosage forms provided herein comprise one or more of an immediate release portions or fractions, fast release portions or fractions, or combinations thereof and an enteric-release portion or fraction, sustained-release portion or fraction, controlled-release portion or fraction, extended-release portion or fraction, pulsatile-release portion or fraction, timed-release portion or fraction, or combinations thereof.

### Pharmaceutical Compositions

In certain embodiments, provided herein is a pharmaceutical composition comprising at least one steroidal compound (e.g., testosterone, dihydrotestosterone, estradiol, or analogs or prodrugs thereof) and at least one pharmaceutically acceptable carrier. In specific embodiments, the steroidal compound is a steroidal androgen (e.g., testosterone, dihydrotestosterone, or prodrugs thereof). In some embodiments, the steroidal compound is an alkylated, hydroxy-alkylated and/or hydroxy-alkoylated natural steroid (e.g., testosterone alkyl ester, dihydrotestosterone alkyl ester, estradiol alkyl ester, or the like). In certain embodiments, analogs or prodrugs of testosterone include, e.g., esters of testosterone. In specific embodiments, the esters of testosterone include, e.g., alkyl (e.g., straight chain, branched, cyclic, unsaturated, partially saturated, fully saturated and the like) esters of testosterone. Specifically, alkyl esters of testosterone include, by way of non-limiting example, lower alkyl esters (e.g., testosterone C₂-C₁₃ alkyl esters such as testosterone propionate, testosterone enanthate, or testosterone undecanoate), or higher alkyl esters (e.g., testosterone C₁₄₊ alkyl esters such as testosterone palmitate). In further embodiments, the alkyl esters of testosterone include, by way of non-limiting example, cycloalkylalkyl esters (e.g., testosterone cypionate), cycloalkyl esters, and alkylcycloalkyl esters. In more specific embodiments, the testosterone alkyl ester is testosterone undecanoate. In some embodiments, the at least one steroidal compound comprises (1) a testosterone lower alkyl ester (e.g., testosterone propionate, testosterone enanthate, or testosterone undecanoate); and (2) a testosterone higher alkyl ester (e.g., testosterone palmitate). Generally, as used herein, a pharmaceutical composition comprising a steroidal compound includes the disclosure of a pharmaceutical composition comprising one or more steroidal compounds.

In certain embodiments, any pharmaceutical composition described herein comprises a therapeutically effective amount of at least one steroidal compound (e.g., a testosterone alkyl ester, such as testosterone undecanoate). In some embodiments, a therapeutically effective amount of a steroidal compound (e.g., a testosterone alkyl ester, such as testosterone undecanoate) is divided into one or more oral dosage form. In some embodiments, the one or more of the oral dosage forms described herein collectively comprise a therapeutically effective amount of a testosterone alkyl ester (e.g., testosterone undecanoate). Thus, in some embodiments, the therapeutically effective amount of a steroidal compound (e.g., a testosterone alkyl ester, such as testosterone undecanoate) within a pharmaceutical composition described herein may vary when the pharmaceutical composition is administered in combination with another therapy. Furthermore, therapeutically effective amounts of a formulation may depend on the specific formulation within which the at least one steroidal compound is found. For example, in some embodiments, more than one steroidal compound is present in a pharmaceutical composition described herein. Thus, when there is a combination of steroidal compounds, in certain instances one or both of the steroidal compounds present has a therapeutically effective amount that is lower than is required when the steroidal compounds are administered separately or alone. In some embodiments, a pharmaceutical composition described herein further comprises an adjuvant, which, in certain instances, allows for a lower amount of a steroidal compound to be utilized as a therapeutically effective amount.

In certain embodiments, a pharmaceutical composition described herein comprises about 1 mg to about 1.5 g, about 10 mg to about 1000 mg, or about 10 mg to about 200 mg of a steroidal compound (e.g., a testosterone alkyl ester, such as testosterone undecanoate). In specific embodiments, a pharmaceutical composition described herein comprises about 10 mg to about 50 mg, about 15 mg to about 40 mg, about 20 mg, to about 30 mg, or about 25 mg of steroidal compound (e.g., a testosterone alkyl ester, such as testosterone undecanoate). In other embodiments, a pharmaceutical composition described herein comprises about 70 mg to about 150 mg, about 80 mg to about 140 mg, about 90 mg to about 140 mg, about 100 mg to about 130 mg, about 110 mg to about 130 mg, or about 120 mg of a steroidal compound (e.g., a testosterone alkyl ester, such as testosterone undecanoate). In some embodiments, a pharmaceutical composition described herein comprises about 0.1 mg to about 5 mg of a steroidal compound (e.g., a testosterone alkyl ester such as testosterone undecanoate) per kg of an individual to whom the oral dosage form is to be administered. In certain embodiments, a pharmaceutical composition described herein comprises an amount of a steroidal compound (e.g., a testosterone alkyl ester, such as testosterone undecanoate) sufficient to provide about 1 mg to about 1 g, about 5 mg to about 500 mg, about 10 mg to about 300 mg, or about 20 to about 250 mg of a steroidal compound (e.g., a testosterone alkyl ester, such as testosterone undecanoate) to an individual upon once a day, twice a day, three times a day, or four times a day oral administration.

In some embodiments, the at least one pharmaceutically acceptable carrier is any carrier suitable for delivering an efficacious amount of a steroidal compound, e.g., a testosterone alkyl ester, to an individual. In some embodiments, the at least one pharmaceutically acceptable carrier is or comprises a hydrophilic carrier (e.g., a hydrophilic surfactant or hydrophilic additive). In certain embodiments, the at least one pharmaceutically acceptable carrier is a lipophilic carrier (e.g., a lipophilic surfactant or lipophilic additive). In some embodiments, the at least one pharmaceutically acceptable carrier is a hydrophilic carrier (e.g., a hydrophilic surfactant or hydrophilic additive) and a lipophilic carrier (e.g., a lipophilic surfactant or lipophilic additive). In certain embodiments, the hydrophilic carrier is a hydrophilic triglyceride. In specific embodiments, the hydrophilic triglyceride is a polyoxylated castor oil, or a polyoxylated hydrogenated castor oil. In some embodiments, any pharmaceutical composition provided herein consists essentially of a lipophilic carrier or combination of lipophilic carriers. In certain embodiments, any pharmaceutical composition provided herein comprises a lipophilic carrier and less than 10% w/w, less than 5% w/w or is substantially free of a hydrophilic carrier. In certain embodiments, any pharmaceutical composition provided herein comprises a lipophilic carrier and less than 10% w/w, less than 5% w/w or is substantially free of a hydrophilic carrier. In some embodiments, the pharmaceutical composition comprising a carrier (e.g., a hydrophilic carrier and/or a lipophilic carrier), the pharmaceutical composition is a solid, a semi-solid, a gel, a jelly, a paste, or the like. In certain embodiments, e.g., wherein a pharmaceutical composition comprising a hydrophilic carrier and/or a lipophilic carrier, a viscosity enhancing agent or a solidifying agent is utilized to afford a pharmaceutical composition that is a solid, a semi-solid, a gel, a jelly, a paste, or the like. Thus, in certain embodiments, the at least one pharmaceutically acceptable carrier is a hydrophilic carrier (e.g., a hydrophilic surfactant or hydrophilic additive) and a viscosity enhancing or solidifying agent. In certain embodiments, the at least one pharmaceutically acceptable carrier is a lipophilic carrier (e.g., a lipophilic surfactant or lipophilic additive) and a viscosity enhancing or solidifying agent. In some embodiments, the at least one pharmaceutically acceptable carrier is or comprises a hydrophilic carrier (e.g., a hydrophilic surfactant or hydrophilic additive), a lipophilic carrier (e.g., a lipophilic surfactant or lipophilic additive), and a viscosity enhancing or solidifying agent. In some embodiments, the at least one pharmaceutically acceptable carrier is or comprises an amphiphilic or zwitterionic carrier (e.g., an amphiphilic surfactant or amphiphilic additive). In certain embodiments, the pharmaceutically acceptable carrier is any carrier suitable for achieving one or more of the pharmacokinetic and/or pharmacodynamic profiles set forth herein.

Additives useful herein include chemical substances that are generally pharmacologically inactive. Further, the additive may be solid, liquid or semi-solid in nature at about ambient room temperature. Furthermore, the additive may be hydrophilic or lipophilic. In certain instances, a “hydrophilic additive” is a substance that has at least one polar side group in its chemical structure which will attract water; whereas a “lipophilic additive” exhibits a tendency to repel water.

In some embodiments, the hydrophilic or lipophilic additive is contained within the components forming a composition and/or pharmaceutical dosage form thereof. In certain embodiments, the hydrophilic or lipophilic additive is in an encapsulation coat in compositions. Alternatively, the additives can be comprised in the pharmaceutical composition but not as part of the composition itself. Specific, non-limiting examples of additives are described below.

Suitable additives include any additive that can facilitate the processes involving the preparation of a pharmaceutical composition and/or dosage form described herein. In some instances, such additives include those commonly utilized to facilitate the processes involving the preparation of a composition and/or a pharmaceutical dosage form described herein. These processes include agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. In certain instances, the additive is optionally pre-coated or encapsulated. Suitable additives are optionally utilized to influence the drug release from the composition and/or pharmaceutical dosage form.

Suitable additives utilized in various embodiments described herein include, by way of non-limiting example, adsorbing agents, anti-adherents, anticoagulants, antifoaming agents, antioxidants, anti-caking agents, anti-static agents, binders, bile acids, bufferants, bulking agents, chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agent sweeteners, stabilizers, surface area enhancing agents, suspending agent, thickeners, viscosity increasing agents, waxes and mixtures thereof.

Some non-limiting examples of the hydrophilic or lipophilic additives suitable for the current invention are as follows:

Alcohols and/or Polyols (e.g. ethanol, isopropanol, butanol, benzyl alcohol, ethylene glycol, propylene glycol, glycerol, sorbitol, mannitol, dimethyl isosorbide, polyethylene glycol (PEG), fatty acid alcohol, vinyl alcohol polypropylene glycol, polyvinylalcohol, tocopherols, cellulose cyclodextrins, other derivatives, forms, mixtures thereof, or the like); ethers of polyethylene glycols having an average molecular weight of about 200 to about 20,000 (e.g. tetrahydrofurfuryl alcohol PEG ether, methoxy PEG, or the like); Amides (e.g. 2-pyrrolidone, 2-piperidone, F-caprolactam, N-alkylpyrrolidone, N-hydroxyalkylpyrrolidone, N-alkylpiperidone, N-alkylcaprolactam, dimethylacetamide, polyvinylpyrrolidone and the like.); Esters (e.g. ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other additives known in the art, such as dimethyl acetamide, dimethyl isosorbide, N-methyl pyrrolidones, monooctanoin, diethylene glycol monoethyl ether, or the like); Amino acids (e.g. P-aminobenzamidine, sodium glycocholate) mesylate; Amino acids and modified amino acids (e.g. aminoboronic acid derivatives and n-acetylcysteine; Peptides and modified peptides (e.g. bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, amastatin, or the like); Polypeptide protease inhibitors; Mucoadhesive polymers (e.g. polyacrylate derivatives, chitosan, cellulosics, chitosan-EDTA, chitosan-EDTA-antipain, polyacrylic acid, carboxymethyl cellulose etc.); or the like; or combinations thereof.

Some more examples of suitable additives for compositions and/or dosage forms described herein include, by way of non-limiting example, talc, magnesium stearate, silica (e.g. fumed silica, micronized silica, magnesium aluminum silicate etc.) and/or derivatives, polyethylene glycols, surfactants, waxes, oils, cetyl alcohol, polyvinyl alcohol, stearic acid, stearic acid salts, stearic acid derivatives, starch, hydrogenated vegetable oils, hydrogenated castor oils, sodium benzoate, sodium acetate, leucine, PEG, alkyl sulfate salts; acetylated monoglycerides; long-chain alcohols; silicone derivatives; butylated hydroxy toluene (BHT), butylated hydroxyl anisole (BHA), gallic acid, propyl gallate, ascorbic acid, ascorbyl palmitate, 4-hydroxymethyl-2,6-di-tert-butyl phenol, dry starch, dry sugars, polyvinyl pyrrolidones, starch paste, methacrylic copolymers, bentonite, sucrose, polymericcellulose derivatives, shellac, sugar syrup; corn syrup; polysaccharides, acacia, tragacanth, guar gum, xanthan gums; alginates; gelatin; gelatin hydrolysate; agar; sucrose; dextrose; PEG, vinyl pyrrolidone copolymers, poloxamers; pregelatinized starch, sorbitol, glucose); acetic acid, hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, vinegar, pharmaceutically acceptable bases, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamin; salt of a pharmaceutically acceptable cation and an anion; EDTA and EDTA salts; titanium dioxide, food dyes, lakes, natural vegetable colorants, iron oxides, silicates, sulfates, magnesium hydroxide and aluminum hydroxide; halogenated hydrocarbons, trichloroethane, trichloroethylene, dichloromethane, fluorotrichloromethane, diethylether, trehelose, phosphates, citric acid, tartaric acid, gelatin, dextran and mannitol, lactose, mannitol, sodium chloride, potassium chloride, spray-dried lactose, hydrolyzed starches, directly compressible starch, microcrystalline cellulose, cellulosic derivatives, sorbitol, sucrose, sucrose-based materials, calcium sulfate, dibasic calcium phosphate, dextrose, croscarmellose sodium, starch, starch derivatives, clays, gums, cellulose, cellulose derivates, alginates, crosslinked polyvinylpyrrolidone, sodium starch glycolate and microcrystalline cellulose, magnesium oxide, magnesium carbonates; desensitizers, spray-dried flavors, essential oils, ethyl vanillin, styrene/divinyl benzene copolymers, quaternary ammonium compounds, polyethylene glycol, citrate esters (such as triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl sebacate, ascorbic acid, boric acid, sorbic acid, benzoic acid, and salts thereof, parabens, phenols, benzyl alcohol, and quaternary ammonium compounds; alcohols, ketones, esters, chlorinated hydrocarbons water; sweeteners, (e.g. maltose, sucrose, glucose, sorbitol, glycerin and dextrins, aspartame, saccharine, saccharine salts, glycyrrhizin), viscosity modifiers, sugars, polyvinylpyrrolidone, cellulosics, polymers, gums and/or alginates.

Additives can also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based polymers (e.g., ethyl cellulose, methyl cellulose, microcrystalline cellulose, sodium carboxymethyl cellulose, hydroxypropylmethyl cellulose, ethyl cellulose, hydroxypropyl cellulose, HPMC acid succinates, cellulose acetate, cellulose nitrate, cellulose acetate butyrate, cellulose acetate trimellitate, carboxymethylethyl cellulose, hydroxypropylmethyl cellulose phthalate), shellacs; inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there is considerable overlap between the above-listed additives in common usage, since a given hydrophilic or lipophilic additive is often classified differently by different practitioners in the field, or is commonly used for any of several different or overlapping functions. Thus, the above-listed hydrophilic or lipophilic additives should be taken as merely exemplary, and not limiting, of the types of additives that can be included in compositions of the present invention. In certain embodiments, the amounts of such additives are optionally adjusted and/or determined by one skilled in the art, according to the particular properties desired.

In certain embodiments, the at least one pharmaceutically acceptable carrier comprises at least one hydrophilic carrier (e.g., hydrophilic surfactant). In some embodiments, the hydrophilic carrier is a polyoxylated glyceride (e.g., mono-, di-, or tri-glyceride), a polyoxylated vegetable oil, a polyoxylated hydrogenated vegetable oil, a polyoxylated fatty acid (mono-, or di-substituted), combinations thereof, or the like. In certain embodiments, the at least one pharmaceutically acceptable carrier comprises or further comprises a lipophilic carrier. Lipophilic carriers are selected from, by way of non-limiting example, a lipophilic surfactant, a vegetable oil (e.g., castor oil), a fatty acid, a fatty alcohol, a glyceride (e.g., mono-, di-, or tri-glyceride), a hydrogenated vegetable oil, a Vitamin E compound (e.g., dl-α-tocopherol), a triglyceride, a fatty acid, polyoxylated fatty acid, polyoxylated triglyceride, polyoxylated vegetable oil, or combinations thereof. In some embodiments, polyoxylated compounds include polyethoxylated compounds.

In certain embodiments, the at least one hydrophilic carriers make up about 1% to about 99% w/w, about 2% to about 80% w/w, about 2% to about 50% w/w, or about 10% to about 40% w/w of any pharmaceutical composition described herein. In some embodiments, lipophilic carriers make up about 1% w/w to about 99% w/w, about 2% to about 80% w/w, about 10% w/w to about 80% w/w, about 30% w/w, to about 80% w/w, or about 40% to about 80% w/w of any pharmaceutical composition described herein.

In specific embodiments, provided herein is a pharmaceutical composition (e.g., a delayed release dosage form) comprising a hydrophilic carrier. In more specific embodiments, the hydrophilic carrier is or comprises a polyoxylated vegetable oil (e.g., a polyoxylated, hydrogenated vegetable oil). In still more specific embodiments, a polyoxylated vegetable oil is a polyoxylated castor oil (e.g., a polyoxylated, hydrogenated castor oil). In certain embodiments, the lipidic and/or lipophilic carrier is not a C₆-C₁₈ fatty acid. In some embodiments, the lipophilic carrier is a C₂₀₊ fatty acid. In some embodiments, the lipidic and/or lipophilic carrier is not a fatty acid or an un-modified (e.g., non-polyoxylated) vegetable oil. In more specific embodiments, the lipidic and/or lipophilic carrier is not oleic acid or castor oil. In certain specific embodiments provided herein is a pharmaceutical composition (e.g., a delayed release dosage form) comprising an amphiphilic carrier. In more specific embodiments, the amphiphilic carrier is or comprises a zwitterionic choline (e.g., phosphatidylcholine). In some specific embodiments, provided herein is a pharmaceutical composition (e.g., a delayed release dosage form) comprising a lipophilic carrier. In more specific embodiments, the lipophilic carrier is or comprises, by way of non-limiting example, a mono-, di- or triglyceride (e.g., glycerol monolinoleate).

In some embodiments, the at least one pharmaceutically acceptable carrier comprises at least one hydrophilic carrier, and at least one lipidic and/or lipophilic carrier. In further embodiments, the at least one pharmaceutically acceptable carrier comprises at least one hydrophilic carrier, at least one lipidic and/or lipophilic carrier, and at least one viscosity enhancer or solidifying agent. In some embodiments, the solidifying agent is a polyethylene glycol (e.g., a high molecular weight polyethylene glycol, such as PEG 8000). In specific embodiments, a pharmaceutical composition described herein comprises, along with a steroidal agent (e.g., a testosterone alkyl ester), a hydrogenated and polyoxylated castor oil and a polyethylene glycol. In more specific embodiments, the pharmaceutical composition comprising a hydrogenated and polyoxylated castor oil and a polyethylene glycol further comprises an additional lipidic and/or lipophilic carrier. In some embodiments, the additional lipidic and/or lipophilic carrier is a monoglyceride, a diglyceride, a Vitamin E compound, or a combination thereof.

In certain embodiments, pharmaceutical compositions described herein include oral dosage forms or delayed release oral dosage forms of any of Tables A to Q. In Tables A to Q, approximate weight percentages of the compositions formulated into the capsules are provided. In specific embodiments, the steroidal compound of any of Capsules A1 to Q2 comprises an alkyl ester testosterone (e.g., testosterone undecanoate). In certain instances, provided in the tables are non-limiting grades and/or sources of components utilized. Disclosure provided in Tables A to Q is not limited to the grades and/or sources described.

TABLE A

| | Capsule A1 | Capsule A2 |
|---|---|---|
| Component | % w/w | % w/w |
| Steroidal Compound (~10-1000 mg) | 1-50 | 10-30 |
| Hydrophilic Carrier | 1-90 | 10-30 |
| Lipophilic Carrier | 1-90 | 40-70 |
| Solidifying Agent (additive) | 1-20 | 5-10 |

TABLE B

| | Capsule B1 | Capsule B2 |
|---|---|---|
| Component | % w/w | % w/w |
| Testosterone undecanoate (~10-1000 mg) | 1-50 | 15 |
| Polyoxyl 40 Hydrogenated Castor Oil, NF | 1-50 | 16 |
| Glycerol Monolinoleate, NF (Maisine 35-1) | 30-90 | 63 |
| Polyethylene Glycol 8000, USP | 1-20 | 6 |

TABLE C

| | Capsule C1 | |
|---|---|---|
| Component | % w/w | Capsule C2 |
| Testosterone undecanoate (~10-1000 mg) | 1-50 | 25 |
| Polyoxyl 35 Castor Oil, NF | 1-50 | 21 |
| Vitamin E, USP (d,l-α-tocopherol) | 30-90 | 48 |
| Polyethylene Glycol 8000, USP | 1-20 | 6 |

TABLE D

| | Capsule D1 | Capsule D2 |
|---|---|---|
| Component | % w/w | % w/w |
| Steroidal Compound (~10-1000 mg) | 15 | 10-30 |
| Lauryl macrogol glyceride (Gelucire 44/14) | 51 | 20-90 |
| Stearoyl macrogol glyceride (Gelucire 50/13) | 34 | 10-90 |

TABLE E

| | Capsule E1 | Capsule E2 |
|---|---|---|
| Component | % w/w | % w/w |
| Steroidal Compound (~10-1000 mg) | 20 | 10-30 |
| C8-C18 macrogol glyceride (Gelucire 43/01) | 35 | 10-70 |
| Polyglyceryl-3-oleate (Caprol 3GO) | 45 | 5-60 |

TABLE F

| | Capsule F1 | Capsule F2 |
|---|---|---|
| Component | % w/w | % w/w |
| Steroidal Compound (~10-1000 mg) | 15 | 10-25 |
| Lauryl macrogol glyceride (Gelucire 44/14) | 40 | 5-80 |
| Vitamin E | 30 | 2-60 |
| Hypromellose (Methocel K100 M LV, CR) | 15 | 5-25 |

TABLE G

| | Capsule G1 | Capsule G2 |
|---|---|---|
| Component | % w/w | % w/w |
| Steroidal Compound (~10-1000 mg) | 15 | 10-30 |
| PEG-40 hydrogenated Castor Oil | 60 | 5-80 |
| (Cremophor ® RH40) | | |
| Polyethylene glycol 8000 | 15 | 5-40 |
| Hypromellose (Methocel K100 M LV, CR) | 10 | 5-25 |

TABLE H

| | Capsule H1 | Capsule H2 |
|---|---|---|
| Component | % w/w | % w/w |
| Steroidal Compound (~10-1000 mg) | 15 | 10-30 |
| Corn Glycerides (Maisine 35-1) | 60 | 5-90 |
| Polyethylene glycol 8000 | 20 | 5-70 |

TABLE I

| | Capsule I1 | Capsule I2 |
|---|---|---|
| Component | % w/w | % w/w |
| Steroidal Compound (~10-1000 mg) | 25 | 10-30 |
| PEG-40 hydrogenated Castor Oil | 15 | 5-80 |
| (Cremophor ® RH40) | | |
| Vitamin E | 20 | 2-60 |
| Corn Glycerides (Maisine 35-1) | 30 | 5-50 |
| Polyethylene Glycol 8000 | 10 | 5-20 |

TABLE J

| | Capsule J1 | Capsule J2 |
|---|---|---|
| Component | % w/w | % w/w |
| Steroidal Compound (~10-1000 mg) | 15 | 10-30 |
| Hydrogenated vegetable oil | 50 | 2-80 |
| Polyethylene glycol 8000 | 35 | 2-80 |

TABLE K

| | Capsule K1 | Capsule K2 |
|---|---|---|
| Component | % w/w | % w/w |
| Steroidal Compound (~10-1000 mg) | 50 | 30-60 |
| Corn Glycerides (Maisine 35-1) | 50 | 30-60 |

TABLE L

| | Capsule L1 | Capsule L2 |
|---|---|---|
| Component | % w/w | % w/w |
| Steroidal Compound (~10-1000 mg) | 40 | 30-60 |
| Fish Oil | 50 | 30-60 |
| Vitamin E | 10 | 3-15 |

TABLE M

| | Capsule M1 | Capsule M2 |
|---|---|---|
| Component | % w/w | % w/w |
| Steroidal Compound (~10-1000 mg) | 40 | 30-60 |
| Omega-3-acid esters | 50 | 30-60 |
| Polyethylene glycol 8000 | 5 | 3-15 |

TABLE N

| | Capsule N1 | Capsule N2 |
|---|---|---|
| Component | % w/w | % w/w |
| Testosterone undecanoate | 5-30 | 10-20 |
| Polyoxyl 40 Hydrogenated Castor Oil, NF | 5-30 | 10-20 |
| Glyceryl Monolinoleate, NF (Maisine 35-1) | 50-90 | 55-70 |
| Polyethylene Glycol 8000, USP | 1-15 | 3-8 |

TABLE O

| | Capsule O1 | Capsule O2 |
|---|---|---|
| Component | % w/w | % w/w |
| Testosterone undecanoate | 10-40 | 20-30 |
| Polyoxyl 35 Castor Oil, NF | 10-30 | 15-25 |
| Vitamin E, USP (d,l-α-tocopherol) | 30-70 | 40-55 |
| Polyethylene Glycol 8000, USP | 1-15 | 3-8 |

TABLE P

| | Capsule P1 | Capsule P2 |
|---|---|---|
| Component | % w/w | % w/w |
| Testosterone undecanoate | 10-40 | 20-25 |
| Vitamin E Polyethylene Glycol | 10-40 | 20-25 |
| Succinate, NF | | |
| Vitamin E, USP (d,l-tocopherol) | 15-60 | 30-40 |
| Polyethylene Glycol 8000, USP | 1-10 | 2-6 |
| Hypromellose (100 cP, K100 Premium LV) | 5-40 | 15-25 |

TABLE Q

| | Capsule Q1 | Capsule Q2 |
|---|---|---|
| Component | % w/w | % w/w |
| Testosterone undecanoate | 10-40 | 20-25 |
| Vitamin E Polyethylene Glycol | 10-40 | 20-25 |
| Succinate, NF | | |
| Vitamin E, USP (d,l-tocopherol) | 15-60 | 30-40 |
| Polyethylene Glycol 8000, USP | 1-10 | 2-6 |
| Hypromellose (4,000 cP, K4M) | 5-40 | 15-25 |

In certain embodiments, any pharmaceutical composition described herein, e.g., a pharmaceutical composition of any of Tables A to Q can be prepared by (i) combining and heating all ingredients until a molten mixture is obtained (e.g., 50-70° C.); and (ii) encapsulating an amount of molten mixture comprising a select dose (e.g., a therapeutically effective amount or a partial dose of a therapeutically effective amount) of steroidal compound to obtain an oral dosage form. In certain instances, the molten mixture is spray-congealed to obtain beads. In some instances, the molten mixture is sprayed onto inert cores (e.g., sugar spheres) to obtain coated cores. In certain embodiments, such beads, cores, or similar forms are encapsulated or otherwise formulated to provide an oral dosage form. In some instances, the molten mixture is admixed, uniformly dispersed, or granulated over a carrier and compressed into a tablet dosage form. In certain embodiments, prior to compression, the molten mixture/carrier composition is further mixed with one or more pharmaceutical aid including, by way of non-limiting example, glidants, lubricants, binders, or the like. In some embodiments, the carrier is a therapeutically inert carrier such as, by way of non-limiting example, microcrystalline cellulose, starch, lactose, or the like.

In some embodiments, compositions described herein (e.g., compositions set forth in Tables K to M), are optionally filled into a delayed release capsule or shell, or are otherwise coated or encapsulated with a delayed release coat.

### Pharmacokinetics and Pharmacodynamics

Provided in certain embodiments herein are androgen therapies (e.g., testosterone undecanoate therapies), pharmaceutical compositions and oral dosage forms that provide a plasma Cₘₐₓ of testosterone that is less than 1500 ng/dL in at least 85% of a population of individuals (following administration of a single dose and/or in the steady state) when administered to a population of individuals. In some embodiments the androgen therapies (e.g., testosterone undecanoate therapies), pharmaceutical compositions or oral dosage forms provide a plasma Cₘₐₓ of testosterone that is less than 1800 ng/dL in at least 95% of a population of individuals (following administration of a single dose and/or in the steady state) when administered to a population of individuals (e.g., adult and/or pubescent human males). In some embodiments the androgen therapies (e.g., testosterone undecanoate therapies), pharmaceutical compositions or oral dosage forms provide a plasma Cₘₐₓ of testosterone that is less than 2500 ng/dL in all or substantially all individuals (following administration of a single dose and/or in the steady state) when administered to a population of individuals (e.g., adult and/or pubescent human males). In some embodiments, the androgen therapies (e.g., testosterone undecanoate therapies), pharmaceutical compositions and oral dosage forms provides a plasma Cₘₐₓ of testosterone that is less than 1500 ng/dL in at least 85% and less than 1800 ng/dL in at least 95% of a population of individuals (following administration of a single dose and/or in the steady state) when administered to a population of individuals (e.g., adult and/or pubescent human males). In certain embodiments, the androgen therapies (e.g., testosterone undecanoate therapies), pharmaceutical compositions and oral dosage forms provides a plasma Cₘₐₓ of testosterone that is less than 1500 ng/dL in at least 85%, less than 1800 ng/dL in at least 95%, and less than 2500 ng/dL in at least 99% of a population of individuals (following administration of a single dose and/or in the steady state) when administered to a population of individuals (e.g., adult and/or pubescent human males). In some embodiments, the androgen therapies (e.g., testosterone undecanoate therapies), pharmaceutical compositions and oral dosage forms provides a plasma Cₘₐₓ of testosterone that is less than 1500 ng/dL in at least 85%, and less than 2500 ng/dL in at least 99% of a population of individuals (following administration of a single dose and/or in the steady state) when administered to a population of individuals (e.g., adult and/or pubescent human males). In some embodiments, the androgen therapies (e.g., testosterone undecanoate therapies), pharmaceutical compositions and oral dosage forms provides a plasma Cₘₐₓ of testosterone that is less than 1800 ng/dL in at least 95%, and less than 2500 ng/dL in at least 99% of a population of individuals (following administration of a single dose and/or in the steady state) when administered to a population of individuals (e.g., adult and/or pubescent human males). In some embodiments, as used in any description herein, individuals are adult humans. In specific embodiments, the adult humans are adult male humans. In certain embodiments, the individuals are adult hypogonadal adult male humans. Plasma concentrations described herein are optionally obtained by administering a composition described herein to human males, e.g., hypogonadal human males. In other instances, plasma concentrations are optionally obtained by administering the composition to testosterone deficient human subjects (e.g., postmenopausal women) who provide a population representative of the effects of testosterone therapy on individuals with low levels of testosterone. Clin. Endocrinology 2007, 66(4):570-85.

Provided in some embodiments herein are pharmaceutical compositions that provide a Cₘᵢₙ that is about 10 ng/dL or greater and a Cₘₐₓ that is about 100 ng/dL or less in at least 50%, at least 60%, at least 70%, at least 80%, at least 90%, at least 95% of adult female humans (e.g., postmenopausal or otherwise androdeficient female humans) when administered to a population of adult female humans (following administration of a single dose and/or in the steady state). Provided in some embodiments herein are pharmaceutical compositions that provide a Cₘᵢₙ that is about 12 ng/dL or greater and a Cₘₐₓ that is about 82 ng/dL or less in at least 50%, at least 60%, at least 70%, at least 80%, at least 90%, at least 95% of adult female humans (e.g., postmenopausal or otherwise androdeficient female humans) when administered to a population of adult female humans (following administration of a single dose and/or in the steady state). In some embodiments, the adult female humans are sexually dysfunctional adult female humans. In certain embodiments, the adult female humans are postmenopausal female humans.

Pharmaceutical compositions and oral dosage forms described herein are formulated, in various embodiments, to achieve the pharmacokinetic and pharmacodynamic profiles herein in any suitable manner. In certain instances, the desired pharmacokinetic and/or pharmacodynamic profile described herein are obtained via the modification of dosage form, the at least one pharmaceutically acceptable carrier, the amount of steroidal compound (e.g., a testosterone alkyl ester, such as testosterone undecanoate) present, combinations thereof, or the like. In certain embodiments, the population of individuals is one, one or more, two or more, or a statistically significant number of individuals.

Provided in certain embodiments herein are androgen therapies (e.g., testosterone undecanoate therapies), pharmaceutical compositions or oral dosage forms described herein that provide or are formulated to provide a plasma concentration of testosterone at steady state that is between about 200 ng/dL and 1300 ng/dL in at least 75%, at least 80%, at least 85%, at least 90%, at least 95%, or at least 99% of a population of individuals when administered to the population of individuals. In some embodiments androgen therapies (e.g., testosterone undecanoate therapies), pharmaceutical compositions or oral dosage forms described herein provide or are formulated to provide a plasma concentration of testosterone at steady state that is between about 200 ng/dL and 1100 ng/dL in at least 75%, at least 80%, at least 85%, at least 90%, at least 95%, or at least 99% of a population of individuals when administered to the population of individuals. In certain embodiments pharmaceutical compositions or oral dosage forms described herein provide or are formulated to provide a plasma concentration of testosterone at steady state that is between about 300 ng/dL and 1000 ng/dL in at least 50%, at least 60%, at least 70%, at least 75%, at least 80%, at least 85%, at least 90%, at least 95%, or at least 99% of a population of individuals when administered to the population of individuals.

In some embodiments, a pharmaceutical composition or oral dosage form described herein (e.g., for use in a steroidal, such as testosterone undecanoate, therapy) is formulated such that a single administration of the pharmaceutical composition or oral dosage form provides a mean plasma Cₘₐₓ of testosterone that is about 19 ng/mL or less, about 18 ng/mL or less, about 17 ng/mL or less, about 16 ng/mL or less, about 15 ng/mL or less, about 14 ng/mL or less, about 5 ng/mL to about 19 ng/mL, about 5 ng/mL to about 18 ng/mL, about 5 ng/mL to about 17 ng/mL, about 5 ng/mL to about 16 ng/mL, about 5 ng/mL to about 15 ng/mL, about 5 ng/mL to about 14 ng/mL, about 7 ng/mL to about 19 ng/mL, about 7 ng/mL to about 18 ng/mL, about 7 ng/mL to about 17 ng/mL, about 7 ng/mL to about 16 ng/mL, about 7 ng/mL to about 15 ng/mL, about 7 ng/mL to about 14 ng/mL, about 10 ng/mL to about 19 ng/mL, about 10 ng/mL to about 18 ng/mL, about 10 ng/mL to about 17 ng/mL, about 10 ng/mL to about 16 ng/mL, about 10 ng/mL to about 15 ng/mL, or about 10 ng/mL to about 14 ng/mL. In specific embodiments, an oral dosage form described herein is formulated such that a single administration of the oral dosage form provides a mean plasma Cₘₐₓ of testosterone that is about 15 ng/mL or less, about 19 ng/mL or less, about 5 ng/mL to about 19 ng/mL, or about 5 ng/mL to about 15 ng/mL. In certain embodiments, a pharmaceutical composition or oral dosage form described herein is formulated such that a single administration of the pharmaceutical composition or oral dosage form provides a mean plasma Cₘₐₓ of dihydrotestosterone that is about 4.5 ng/mL or less, about 4.3 ng/mL or less, about 4.2 ng/mL or less, about 4.1 ng/mL or less, about 4 ng/mL or less, about 3.9 ng/mL or less, about 3.8 ng/mL or less, about 3.7 ng/mL or less, about 3.6 ng/mL or less, about 3.5 ng/mL or less, about 1.5 ng/mL to about 4.5 ng/mL, about 1.5 ng/mL to about 3.9 ng/mL, about 1.5 ng/mL to about 3.8 ng/mL, about 1.5 ng/mL to about 3.7 ng/mL, about 1.5 ng/mL to about 3.6 ng/mL, about 1.5 ng/mL to about 3.5 ng/mL, about 2.0 ng/mL to about 4.5 ng/mL, about 2.0 ng/mL to about 3.9 ng/mL, about 2.0 ng/mL to about 3.8 ng/mL, about 2.0 ng/mL to about 3.7 ng/mL, about 2.0 ng/mL to about 3.6 ng/mL, about 2.0 ng/mL to about 3.5 ng/mL, about 2.5 ng/mL to about 3.9 ng/mL, about 2.5 ng/mL to about 3.8 ng/mL, about 2.5 ng/mL to about 3.7 ng/mL, about 2.5 ng/mL to about 3.6 ng/mL, or about 2.5 ng/mL to about 3.5 ng/mL. In specific embodiments, a pharmaceutical composition or oral dosage form described herein is formulated such that a single administration of the pharmaceutical composition or oral dosage form provides a mean plasma Cₘₐₓ of dihydrotestosterone that is about 3.6 ng/mL or less upon oral administration. In certain embodiments, a pharmaceutical composition or oral dosage form described herein is formulated such that a single administration of the pharmaceutical composition or oral dosage form provides a mean plasma Cₘₐₓ of testosterone alkyl ester (e.g., testosterone undecanoate) that is about 400 ng/mL or less, about 380 ng/mL or less, about 360 ng/mL or less, about 340 ng/mL or less, about 320 ng/mL or less, about 300 ng/mL or less, or about 280 ng/mL or less, about 100 ng/mL to about 400 ng/mL, about 100 ng/mL to about 380 ng/mL, about 100 ng/mL to about 360 ng/mL, about 100 ng/mL to about 340 ng/mL, about 100 ng/mL to about 320 ng/mL, about 100 ng/mL to about 300 ng/mL, about 100 ng/mL to about 280 ng/mL, about 150 ng/mL to about 400 ng/mL, about 150 ng/mL to about 380 ng/mL, about 150 ng/mL to about 360 ng/mL, about 150 ng/mL to about 340 ng/mL, about 150 ng/mL to about 320 ng/mL, about 150 ng/mL to about 300 ng/mL, about 150 ng/mL to about 280 ng/mL, about 200 ng/mL to about 400 ng/mL, about 200 ng/mL to about 380 ng/mL, about 200 ng/mL to about 360 ng/mL, about 200 ng/mL to about 340 ng/mL, about 200 ng/mL to about 320 ng/mL, about 200 ng/mL to about 300 ng/mL, or about 200 ng/mL to about 280 ng/mL. In specific embodiments, a pharmaceutical composition or oral dosage form described herein is formulated such that a single administration of the pharmaceutical composition or oral dosage form provides a mean plasma Cₘₐₓ of testosterone undecanoate that is about 380 ng/mL or less upon oral administration. In some embodiments, a pharmaceutical composition or oral dosage form described herein (e.g., for use in a steroidal, such as testosterone undecanoate, therapy) is formulated such that a single administration of the pharmaceutical composition or oral dosage form provides a mean plasma Cₘₐₓ of testosterone that is about 5 ng/mL to about 15 ng/mL, a mean plasma Cₘₐₓ of dihydrotestosterone that is about 1.5 ng/mL to about 3.8 ng/mL, and a mean plasma Cₘₐₓ of testosterone alkyl ester (e.g., testosterone undecanoate) that is about 100 ng/mL to about 380 ng/mL. In certain embodiments, a pharmaceutical composition or oral dosage form described herein (e.g., for use in a steroidal, such as testosterone undecanoate, therapy) is formulated such that a single administration of the pharmaceutical composition or oral dosage form provides a mean plasma Cₘₐₓ of testosterone that is about 5 ng/mL to about 19 ng/mL, a mean plasma Cₘₐₓ of dihydrotestosterone that is about 1.5 ng/mL to about 4.5 ng/mL, and a mean plasma Cₘₐₓ of testosterone alkyl ester (e.g., testosterone undecanoate) that is about 100 ng/mL to about 380 ng/mL.

In some embodiments, provided herein is a pharmaceutical composition or oral dosage form formulated such that it provides a mean plasma concentration of testosterone that is about 200 ng/dL or less, about 150 ng/dL or less, about 100 ng/dL or less, or about 75 ng/dL or less, about 5 ng/dL to about 200 ng/dL, about 5 ng/dL to about 150 ng/dL, about 5 ng/dL to about 100 ng/dL, about 5 ng/dL to about 75 ng/dL, about 10 ng/dL to about 200 ng/dL, about 10 ng/dL to about 150 ng/dL, about 10 ng/dL to about 100 ng/dL, about 10 ng/dL to about 75 ng/dL, about 15 ng/dL to about 200 ng/dL, about 15 ng/dL to about 150 ng/dL, about 15 ng/dL to about 100 ng/dL, or about 15 ng/dL to about 75 ng/dL 1 hour after a single oral administration. In certain embodiments, provided herein is a pharmaceutical composition or oral dosage form formulated such that it provides a mean plasma concentration of testosterone that is about 500 ng/dL or less, about 400 ng/dL or less, about 300 ng/dL or less, about 200 ng/dL or less, about 150 ng/dL or less, about 5 ng/dL to about 500 ng/dL, about 5 ng/dL to about 400 ng/dL, about 5 ng/dL to about 300 ng/dL, about 5 ng/dL to about 200 ng/dL, about 5 ng/dL to about 150 ng/dL, about 10 ng/dL to about 500 ng/dL, about 10 ng/dL to about 400 ng/dL, about 10 ng/dL to about 300 ng/dL, about 10 ng/dL to about 200 ng/dL, about 10 ng/dL to about 150 ng/dL, about 15 ng/dL to about 500 ng/dL, about 15 ng/dL to about 400 ng/dL, about 15 ng/dL to about 300 ng/dL, about 15 ng/dL to about 200 ng/dL, about 15 ng/dL to about 150 ng/dL 2 hour after a single oral administration. In some embodiments, provided herein is a pharmaceutical composition or oral dosage form formulated such that it provides a mean plasma concentration of testosterone that is about 5 ng/dL to about 150 ng/dL 1 hour after a single oral administration, and about 10 ng/dL to about 500 ng/dL 2 hours after a single oral administration.

In certain embodiments, pharmaceutical compositions described herein comprise or are formulated into one or more oral dosage form described herein. Therefore, in some embodiments, in order to arrive at the targeted plasma concentration (e.g., at a specific concentration at a given time, Cₘₐₓ, Cₘᵢₙ, or the like), a plurality of oral dosage forms described herein are optionally administered. Furthermore, as used herein, a mean plasma concentration (e.g., at a specific concentration at a given time, Cₘₐₓ, Cₘᵢₙ, or the like) is the mean of a plurality of concentration values obtained from the plasma of a plurality of individuals following oral administration of an oral dosage form described herein to the plurality of individuals. In some embodiments, the individuals are adult humans. In specific embodiments, the adult humans are adult male humans. In certain embodiments, the individuals are adult hypogonadal or otherwise androdeficient adult male humans. In some embodiments, the individuals are postmenopausal or otherwise androdeficient adult female humans. Furthermore, it is noted that concentrations of testosterone alkyl ester described herein include the concentration of the one or more testosterone alkyl ester administered.

In some embodiments, provided herein is a pharmaceutical composition or oral dosage form that releases or is formulated to release about 90% or less, about 80% or less, about 70% or less, about 60% or less, about 55% or less, about 50% or less, about 45% or less, about 40% or less, about 35% or less, about 5% to about 90%, about 5% to about 80%, about 5% to about 70%, about 5% to about 60%, about 5% to about 55%, about 5% to about 50%, about 5% to about 45%, about 5% to about 40%, about 5% to about 35%, about 20% to about 90%, about 20% to about 80%, about 20% to about 70%, about 20% to about 60%, about 20% to about 55%, about 20% to about 50%, about 20% to about 45%, about 20% to about 40%, or about 20% to about 35% of the testosterone alkyl ester (e.g., testosterone undecanoate) after 1 hour in an aqueous medium (e.g., in 1 L deionized water comprising 8% w/v Triton X-100). In certain embodiments, provided herein is a pharmaceutical composition or oral dosage form that releases or is formulated to release about 90% or less, about 80% or less, about 70% or less, about 60% or less, about 50% or less, about 40% or less, about 30% or less, about 20% or less, about 2% to about 90%, about 2% to about 80%, about 2% to about 70%, about 2% to about 60%, about 2% to about 50%, about 2% to about 40%, about 2% to about 30%, about 2% to about 20%, about 10% to about 90%, about 10% to about 80%, about 10% to about 70%, about 10% to about 60%, about 10% to about 50%, about 10% to about 40%, about 10% to about 30%, or about 10% to about 20% of the testosterone alkyl ester (e.g., testosterone undecanoate) after 30 minutes in an aqueous medium (e e.g., in 1 L deionized water comprising 8% w/v Triton X-100). In some embodiments, provided herein is a pharmaceutical composition or oral dosage form that releases or is formulated to release about 99% or less, about 98% or less, about 97% or less, about 96% or less, about 95% or less, about 90% or less, about 10% to about 99%, about 10% to about 98%, about 10% to about 97%, about 10% to about 96%, about 10% to about 95%, about 10% to about 90%, about 40% to about 99%, about 40% to about 98%, about 40% to about 97%, about 40% to about 96%, about 40% to about 95%, about 40% to about 90%, about 70% to about 99%, about 70% to about 98%, about 70% to about 97%, about 70% to about 96%, about 70% to about 95%, or about 70% to about 90% of the testosterone alkyl ester (e.g., testosterone undecanoate) after 3 hour in an aqueous medium (e.g., in 1 L deionized water comprising 8% w/v Triton X-100). In some embodiments, provided herein is a pharmaceutical composition or oral dosage form that releases or is formulated to release more than 80% of the testosterone alkyl ester (e.g., testosterone undecanoate) within 12, 10, 8, 6, 5, 4, 3, or 2 hours in an aqueous medium (e.g., in 1 L deionized water comprising 8% w/v Triton X-100). In specific embodiments, provided herein is a pharmaceutical composition or oral dosage form that releases or is formulated to release about 20% or less of the testosterone alkyl ester after 30 minutes, 50% or less of the testosterone alkyl ester (e.g., testosterone undecanoate) after 1 hour and about 95% or less of the testosterone alkyl ester after 3 hours in an aqueous medium (e.g., in 1 L deionized water comprising 8% w/v Triton X-100). In some embodiments, provided herein is a pharmaceutical composition or oral dosage form that releases or is formulated to release about 5% to about 60% of the testosterone alkyl ester (e.g., testosterone undecanoate) after 1 hour, about 2% to about 40% of the testosterone alkyl ester after 30 minutes, and about 10% to about 95% of the testosterone alkyl ester after 2 hours in an aqueous medium (e.g., in 1 L deionized water comprising 8% w/v Triton X-100). In certain specific embodiments, provided herein is a pharmaceutical composition or oral dosage form that releases or is formulated to release about 50% or less of the testosterone alkyl ester (e.g., testosterone undecanoate) after 1 hour, and 80% or less of the testosterone alkyl ester (e.g., testosterone undecanoate) within 2-12 hours (or after 12 hours, 10 hours, 8 hours, 6 hours, 5 hours, 4 hours, 3 hours, or 2 hours) in an aqueous medium (e.g., in 1 L deionized water comprising 8% w/v Triton X-100). In certain instances, the aqueous medium is 1 L deionized water comprising 8% w/v Triton X-100 (e.g., octylphenol ethylene oxide condensate; octoxynol-9; t-octylphenoxypolyethoxyethanol; t-oct-C₆H₄—(OCH₂CH₂)ₓOH, x=9-10; CAS No. 9002-93-1; Triton X-100 was a registered trademark formerly owned by Rohm and Haas Co., but now owned by Union Carbide) at 37±0.5° C. and the pharmaceutical composition or oral dosage form is deposited therein and subjected to a paddle method at 100 rpm and 37±0.5° C. for the designated period of time (USP App 2).

In some embodiments, provided herein is a pharmaceutical composition or oral dosage form that provides or is formulated to provide a testosterone (e.g., in human males, adult human males, pubescent human males, or the like) mean plasma Cₘₐₓ at steady state of about 1550 ng/dL or less, about 1500 ng/dL or less, about 1450 ng/dL or less, about 1400 ng/dL or less, about 1310 ng/dL or less, about 1300 ng/dL or less. In some embodiments, provided herein is a pharmaceutical composition or oral dosage form that provides or is formulated to provide a testosterone (e.g., in human males, adult human males, pubescent human males, or the like) mean plasma Cₘᵢₙ at steady state of about 100 ng/dL or more, about 150 ng/dL or more, about 200 ng/dL or more, about 250 ng/dL or more, or about 300 ng/dL or more. In specific embodiments, provided herein is a pharmaceutical composition or oral dosage form that provides or is formulated to provide a testosterone (e.g., in human males, adult human males, pubescent human males, or the like) mean plasma Cₘᵢₙ at steady state of about 200 ng/dL or more. In certain embodiments, provided herein is a pharmaceutical composition or oral dosage form that provides or is formulated to provide a testosterone (e.g., in human males, adult human males, pubescent human males, or the like) mean plasma concentration that ranges at steady state from about 100 ng/dL to about 1500 ng/dL, about 150 ng/dL to about 1400 ng/dL, about 200 ng/dL to about 1300 ng/dL or about 250 ng/dL to about 1200 ng/dL. In specific embodiments, provided herein is a pharmaceutical composition or oral dosage form that provides or is formulated to provide a testosterone (e.g., in human males, adult human males, pubescent human males, or the like) mean plasma concentration that ranges at steady state from about 200 ng/dL to about 1300 ng/dL.

In some embodiments, provided herein is a pharmaceutical composition or oral dosage form that provides or is formulated to provide a testosterone (e.g., in human females, adult human females, post-menopausal human females, or the like) mean plasma Cₘₐₓ at steady state of about 110 ng/dL or less, 100 ng/dL or less, about 95 ng/dL or less, about 90 ng/dL or less, about 85 ng/dL or less, or about 82 ng/dL or less. In some embodiments, provided herein is a pharmaceutical composition or oral dosage form that provides or is formulated to provide a testosterone (e.g., in human females, adult human females, post-menopausal human females, or the like) mean plasma Cₘᵢₙ at steady state of about 3 ng/dL or more, about 5 ng/dL or more, about 8 ng/dL or more, about 10 ng/dL or more, or about 12 ng/dL or more. In specific embodiments, provided herein is a pharmaceutical composition or oral dosage form that provides or is formulated to provide a testosterone (e.g., in human females, adult human females, post-menopausal human females, or the like) mean plasma Cₘᵢₙ at steady state of about 8 ng/dL or more. In certain embodiments, provided herein is a pharmaceutical composition or oral dosage form that provides or is formulated to provide a testosterone (e.g., in human males, adult human females, pubescent human females, postmenopausal human females, or the like) mean plasma concentration that ranges at steady state from about 5 ng/dL to about 110 ng/dL, about 8 ng/dL to about 100 ng/dL, about 10 ng/dL to about 90 ng/dL or about 12 ng/dL to about 82 ng/dL. In specific embodiments, provided herein is a pharmaceutical composition or oral dosage form that provides or is formulated to provide a testosterone (e.g., in human females, adult human females, post-menopausal human females, or the like) mean plasma concentration that ranges at steady state from about 10 ng/dL to about 90 ng/dL.

Provided in certain embodiments herein is a pharmaceutical composition or oral dosage form that provides or is formulated to provide upon oral administration to an individual (e.g., an androgen deficient human male) a testosterone equivalent (e.g., mass of testosterone that can be derived from a testosterone alkyl ester (e.g., C₂-C₁₃)) dose to mean steady state testosterone Cₘₐₓ ratio of about 500×10⁶ mL or less. In some embodiments, a testosterone equivalent dose to mean steady state testosterone Cₘₐₓ ratio is about 500×10⁶ mL, or less; about 4×10⁵ mL, or more; about 6×10⁵ mL, or more; about 8×10⁵ mL, or more; about 1×10⁶ mL, or more; about 3×10⁶ mL, or more; about 4×10⁶ mL, or more; about 5×10⁶ mL, or more; about 6×10⁶ mL, or more; 500×10⁶ mL, or less; 400×10⁶ mL, or less; 300×10⁶ mL, or less; 250×10⁶ mL, or less; 200×10⁶ mL, or less; 150×10⁶ mL, or less; 100×10⁶ mL, or less; about 25×10⁵ mL, or more; about 100×10⁵ mL, or more; about 250×10⁵ mL, or more; about 500×10⁵ mL, or more; about 4×10⁵ mL to about 500×10⁶ mL; about 4×10⁵ mL to about 400×10⁶ mL; about 4×10⁵ mL to about 300×10⁶ mL; about 4×10⁵ mL to about 250×10⁶ mL; about 4×10⁵ mL to about 200×10⁶ mL; about 4×10⁵ mL to about 150×10⁶ mL; about 20×10⁵ mL to about 500×10⁶ mL; about 20×10⁵ mL to about 400×10⁶ mL; about 20×10⁵ mL to about 300×10⁶ mL; about 20×10⁵ mL to about 250×10⁶ mL; about 20×10⁵ mL to about 200×10⁶ mL; about 20×10⁵ mL to about 150×10⁶ mL; about 50×10⁵ mL to about 500×10⁶ mL; about 50×10⁵ mL to about 400×10⁶ mL; about 50×10⁵ mL to about 300×10⁶ mL; about 50×10⁵ mL to about 250×10⁶ mL; about 50×10⁵ mL to about 200×10⁶ mL; about 50×10⁵ mL to about 150×10⁶ mL; about 200×10⁵ mL to about 500×10⁶ mL; about 200×10⁵ mL to about 400×10⁶ mL; about 200×10⁵ mL to about 300×10⁶ mL; about 200×10⁵ mL to about 250×10⁶ mL; about 200×10⁵ mL to about 200×10⁶ mL; about 200×10⁵ mL to about 150×10⁶ mL; or the like. In some embodiments, a single dose of any oral dosage form or pharmaceutical composition described herein provides, upon oral administration to an individual (e.g., an androgen deficient human male), a ratio testosterone equivalent dose to mean plasma testosterone Cₘₐₓ that is about 500×10⁶ mL or less. In some embodiments, a single administration provides a testosterone equivalent dose to mean testosterone Cₘₐₓ ratio that is about 500×10⁶ mL, or less; about 4×10⁵ mL, or more; 500×10⁶ mL, or less; 400×10⁶ mL, or less; 300×10⁶ mL, or less; 250×10⁶ mL, or less; 200×10⁶ mL, or less; 150×10⁶ mL, or less; 100×10⁶ mL, or less; about 25×10⁵ mL, or more; about 100×10⁵ mL, or more; about 250×10⁵ mL, or more; about 500×10⁵ mL, or more; about 4×10⁵ mL to about 500×10⁶ mL; about 4×10⁵ mL to about 400×10⁶ mL; about 4×10⁵ mL to about 300×10⁶ mL; about 4×10⁵ mL to about 250×10⁶ mL; about 4×10⁵ mL to about 200×10⁶ mL; about 4×10⁵ mL to about 150×10⁶ mL; about 20×10⁵ mL to about 500×10⁶ mL; about 20×10⁵ mL to about 400×10⁶ mL; about 20×10⁵ mL to about 300×10⁶ mL; about 20×10⁵ mL to about 250×10⁶ mL; about 20×10⁵ mL to about 200×10⁶ mL; about 20×10⁵ mL to about 150×10⁶ mL; about 50×10⁵ mL to about 500×10⁶ mL; about 50×10⁵ mL to about 400×10⁶ mL; about 50×10⁵ mL to about 300×10⁶ mL; about 50×10^{h} mL to about 250×10⁶ mL; about 50×10⁵ mL to about 200×10⁶ mL; about 50×10⁵ mL to about 150×10⁶ mL; about 200×10⁵ mL to about 500×10⁶ mL; about 200×10⁵ mL to about 400×10⁶ mL; about 200×10⁵ mL to about 300×10⁶ mL; about 200×10⁵ mL to about 250×10⁶ mL; about 200×10⁵ mL to about 200×10⁶ mL; about 200×10⁵ mL to about 150×10⁶ mL; or the like. In some embodiments, a single administration provides a testosterone equivalent dose to mean dihydroxytestosterone Cₘₐₓ ratio that is about 350×10⁶ mL, or less; about 20×10⁵ mL, or more; 500×10⁶ mL, or less; 400×10⁶ mL, or less; 300×10⁶ mL, or less; 250×10⁶ mL, or less; 200×10⁶ mL, or less; 150×10⁶ mL, or less; 100×10⁶ mL, or less; about 25×10⁵ mL, or more; about 100×10⁵ mL, or more; about 250×10⁵ mL, or more; about 500×10⁵ mL, or more; about 20×10⁵ mL to about 500×10⁶ mL; about 20×10⁵ mL to about 400×10⁶ mL; about 20×10⁵ mL to about 300×10⁶ mL; about 20×10⁵ mL to about 250×10⁶ mL; about 20×10⁵ mL to about 200×10⁶ mL; about 20×10⁵ mL to about 150×10⁶ mL; about 50×10⁵ mL to about 500×10⁶ mL; about 50×10⁵ mL to about 400×10⁶ mL; about 50×10⁵ mL to about 300×10⁶ mL; about 50×10⁵ mL to about 250×10⁶ mL; about 50×10⁵ mL to about 200×10⁶ mL; about 50×10⁵ mL to about 150×10⁶ mL; about 200×10⁵ mL to about 500×10⁶ mL; about 200×10⁵ mL to about 400×10⁶ mL; about 200×10⁵ mL to about 300×10⁶ mL; about 200×10⁵ mL to about 250×10⁶ mL; about 200×10⁵ mL to about 200×10⁶ mL; about 200×10⁵ mL to about 150×10⁶ mL; or the like. In certain instances, a steroid equivalent dose (e.g., testosterone equivalent dose) of a composition or dosage form described herein is the amount of steroid compound (e.g., testosterone) present (e.g., the steroidal portion of a steroidal compound, such as a testosterone alkyl ester) in the composition or dosage form and can be determined by calculating, e.g., (mass testosterone/mass testosterone alkyl ester)*amount of testosterone alkyl ester in the composition or dosage form.

Provided in certain embodiments herein is a pharmaceutical composition or oral dosage form that provides or is formulated to provide a difference between the mean plasma Cₘₐₓ of testosterone at steady state and mean plasma Cₘᵢₙ of testosterone at steady state that is about 20 ng/mL or less, about 19 ng/mL or less, about 18 ng/mL or less, about 17 ng/mL or less, about 16 ng/mL or less, about 15 ng/mL or less, about 14 ng/mL or less, about 13 ng/mL or less, about 12 ng/mL or less, about 11 ng/mL or less, about 10.8 ng/mL or less, about 2 to about 20 ng/mL, about 2 to about 18 ng/mL, about 2 to about 16 ng/mL, about 2 to about 15 ng/mL, about 2 to about 14 ng/mL, about 2 to about 13 ng/mL, about 2 to about 12 ng/mL, about 2 to about 11 ng/mL, about 5 to about 15 ng/mL, about 5 to about 14 ng/mL, about 5 to about 13 ng/mL, about 5 to about 12 ng/mL, or about 5 to about 11 ng/mL. In specific embodiments, the pharmaceutical composition or oral dosage form provides or is formulated to provide a difference between the mean plasma Cₘₐₓ of testosterone at steady state and mean plasma Cₘᵢₙ of testosterone at steady state that is about 11 ng/mL or less. Furthermore, in some embodiments, provided herein is a pharmaceutical composition or oral dosage form provided herein provides or is formulated to provide a difference between the mean plasma Cₘₐₓ and the mean Cₘᵢₙ of testosterone alkyl ester (e.g., testosterone undecanoate) is about 275 ng/mL or less, about 260 ng/mL or less, about 250 ng/mL or less, about 240 ng/mL or less, about 230 ng/mL or less, about 225 ng/mL or less, about 220 ng/mL or less, about 210 ng/mL or less, about 200 ng/mL or less, about 190 ng/mL or less, or about 180 ng/mL or less. In specific embodiments, provided herein is a pharmaceutical composition or oral dosage form provided herein provides or is formulated to provide a difference between the mean plasma Cₘₐₓ and mean plasma Cₘᵢₙ of testosterone alkyl ester (e.g., testosterone undecanoate) is about 200 ng/mL or less. In specific embodiments, provided herein is a pharmaceutical composition or oral dosage form provided herein provides or is formulated to provide a difference between the mean plasma Cₘₐₓ and mean plasma Cₘᵢₙ of testosterone alkyl ester (e.g., testosterone undecanoate) is about 275 ng/mL or less.

In some embodiments, provided herein is a pharmaceutical composition or oral dosage form that is formulated such that it provides, following a single oral administration, a mean plasma AUC_{0-∞} concentration of testosterone of about 120 ng·h/mL or less, about 110 ng·h/mL or less, about 100 ng·h/mL or less, about 90 ng·h/mL or less, about 80 ng·h/mL or less, about 70 ng·h/mL or less, about 60 ng·h/mL or less, about 20 ng·h/mL to about 110 ng·h/mL, about 20 ng·h/mL to about 100 ng·h/mL, about 20 ng·h/mL to about 90 ng·h/mL, about 20 ng·h/mL to about 80 ng·h/mL, about 20 ng·h/mL to about 70 ng·h/mL, about 20 ng·h/mL to about 60 ng·h/mL, about 30 ng·h/mL to about 110 ng·h/mL, about 30 ng·h/mL to about 100 ng·h/mL, about 30 ng·h/mL to about 90 ng·h/mL, about 30 ng·h/mL to about 80 ng·h/mL, about 30 ng·h/mL to about 70 ng·h/mL, about 30 ng·h/mL to about 60 ng·h/mL, about 40 ng·h/mL to about 110 ng·h/mL, about 40 ng·h/mL to about 100 ng·h/mL, about 40 ng·h/mL to about 90 ng·h/mL, about 40 ng·h/mL to about 80 ng·h/mL, about 40 ng·h/mL to about 70 ng·h/mL, about 40 ng·h/mL to about 60 ng·h/mL, about 50 ng·h/mL to about 110 ng·h/mL, about 50 ng·h/mL to about 100 ng·h/mL, about 50 ng·h/mL to about 90 ng·h/mL, about 50 ng·h/mL to about 80 ng·h/mL, about 50 ng·h/mL to about 70 ng·h/mL, about 60 ng·h/mL to about 110 ng·h/mL, about 60 ng·h/mL to about 100 ng·h/mL, about 60 ng·h/mL to about 90 ng·h/mL, or about 60 ng·h/mL to about 80 ng·h/mL. In certain embodiments, provided herein is a pharmaceutical composition or oral dosage form that is formulated such that, following a single oral administration, it provides a mean plasma AUC_{0-∞} concentration of dihydrotestosterone of about 50 ng·h/mL or less, about 45 ng·h/mL or less, about 40 ng·h/mL or less, about 35 ng·h/mL or less, about 30 ng·h/mL or less, about 25 ng·h/mL or less, about 20 ng·h/mL or less, about 10 ng·h/mL to about 50 ng·h/mL, about 10 ng·h/mL to about 45 ng·h/mL, about 10 ng·h/mL to about 40 ng·h/mL, about 10 ng·h/mL to about 35 ng·h/mL, about 10 ng·h/mL to about 30 ng·h/mL, about 10 ng·h/mL to about 25 ng·h/mL, about 10 ng·h/mL to about 20 ng·h/mL, about 15 ng·h/mL to about 50 ng·h/mL, about 15 ng·h/mL to about 45 ng·h/mL, about 15 ng·h/mL to about 40 ng·h/mL, about 15 ng·h/mL to about 35 ng·h/mL, about 15 ng·h/mL to about 30 ng·h/mL, about 15 ng·h/mL to about 25 ng·h/mL, about 20 ng·h/mL to about 50 ng·h/mL, about 20 ng·h/mL to about 45 ng·h/mL, about 20 ng·h/mL to about 40 ng·h/mL, about 20 ng·h/mL to about 35 ng·h/mL, or about 20 ng·h/mL to about 30 ng·h/mL. In some embodiments, provided herein is a pharmaceutical composition or oral dosage form that is formulated such that, following a single oral administration, it provides a mean plasma AUC_{0-∞} concentration of testosterone alkyl ester (e.g., the one or more testosterone alkyl ester compounds, such as testosterone undecanoate, found in the composition or dosage form) of about 1200 ng·h/mL or less, about 1100 ng·h/mL or less, about 1000 ng·h/mL or less, about 900 ng·h/mL or less, about 850 ng·h/mL or less, about 800 ng·h/mL or less, about 750 ng·h/mL or less, about 100 ng·h/mL to about 1200 ng·h/mL, about 100 ng·h/mL to about 1100 ng·h/mL, about 100 ng·h/mL to about 1000 ng·h/mL, about 100 ng·h/mL to about 900 ng·h/mL, about 100 ng·h/mL to about 850 ng·h/mL, about 100 ng·h/mL to about 800 ng·h/mL, about 100 ng·h/mL to about 750 ng·h/mL, about 150 ng·h/mL to about 1200 ng·h/mL, about 150 ng·h/mL to about 1100 ng·h/mL, about 150 ng·h/mL to about 1000 ng·h/mL, about 150 ng·h/mL to about 900 ng·h/mL, about 150 ng·h/mL to about 850 ng·h/mL, about 150 ng·h/mL to about 800 ng·h/mL, about 150 ng·h/mL to about 750 ng·h/mL, about 200 ng·h/mL to about 1200 ng·h/mL, about 200 ng·h/mL to about 1100 ng·h/mL, about 200 ng·h/mL to about 1000 ng·h/mL, about 200 ng·h/mL to about 900 ng·h/mL, about 200 ng·h/mL to about 850 ng·h/mL, about 200 ng·h/mL to about 800 ng·h/mL, about 200 ng·h/mL to about 750 ng·h/mL, about 250 ng·h/mL to about 1200 ng·h/mL, about 250 ng·h/mL to about 1100 ng·h/mL, about 250 ng·h/mL to about 1000 ng·h/mL, about 250 ng·h/mL to about 900 ng·h/mL, about 250 ng·h/mL to about 850 ng·h/mL, about 250 ng·h/mL to about 800 ng·h/mL, about 250 ng·h/mL to about 750 ng·h/mL, about 300 ng·h/mL to about 1200 ng·h/mL, about 300 ng·h/mL to about 1100 ng·h/mL, about 300 ng·h/mL to about 1000 ng·h/mL, about 300 ng·h/mL to about 900 ng·h/mL, about 300 ng·h/mL to about 850 ng·h/mL, about 300 ng·h/mL to about 800 ng·h/mL, or about 300 ng·h/mL to about 750 ng·h/mL.

Provided in certain embodiments herein is any oral dosage form or pharmaceutical composition described herein that when a single dose is administered to an individual provides a testosterone equivalent dose to mean testosterone AUC_{0-∞} ratio of about 500×10³ mL/h or less. In some embodiments, the testosterone equivalent dose to mean AUC_{0-∞} ratio is about 20×10³ mL/h, or more; about 30×10³ mL/h, or more; about 40×10³ mL/h, or more; about 50×10³ mL/h, or more; about 60×10³ mL/h, or more; about 80×10³ mL/h, or more; about 100×10³ mL/h, or more; about 600×10³ mL/h, or less; about 400×10³ mL/h, or less; about 350×10³ mL/h, or less; about 250×10³ mL/h, or less; about 200×10³ mL/h, or less; about 150×10³ mL/h, or less; about 10 to about 600×10³ mL/h; about 20 to about 500×10³ mL/h; about 30 to about 450×10³ mL/h; about 20 to about 400×10³ mL/h; about 50 to about 300×10³ mL/h; about 50 to about 200×10³ mL/h; or the like.

In certain embodiments, a pharmaceutical composition or oral dosage form described herein achieves steady state upon administration in any manner suitable to achieve the steady state, e.g., once a day, twice a day, three times a day, four times a day, or the like. In specific embodiments, steady state is achieved after a period of time of b.i.d. oral administration (e.g., every 12 hours) of an oral dosage form described herein. In certain embodiments, steady state is obtained after, e.g., 2 days, 3 days, 4 days, 5 days, 6 days, 7 days, 2 weeks or longer, if or as necessary. In specific embodiments, steady state is obtained after b.i.d. oral administration for 5-7 days. Moreover, steady state plasma concentrations of testosterone, testosterone alkyl ester (e.g., testosterone undecanoate), and dihydrotestosterone are obtained, in certain instances, by administration of pharmaceutical compositions comprising about 1 mg to about 1 g, or about 10 mg to about 200 mg of a steroidal compound (e.g., a testosterone alkyl ester, such as testosterone undecanoate). In specific embodiments, a pharmaceutical composition (e.g., for administration to a human male) comprises about 10 mg to about 50 mg, about 15 mg to about 40 mg, about 20 mg, to about 30 mg, or about 25 mg of steroidal compound (e.g., a testosterone alkyl ester, such as testosterone undecanoate). In other embodiments, a pharmaceutical composition (e.g., for administration to a human male) comprises about 70 mg to about 150 mg, about 80 mg to about 140 mg, about 90 mg to about 140 mg, about 100 mg to about 130 mg, about 110 mg to about 130 mg, about 80 mg, or about 120 mg of a steroidal compound (e.g., a testosterone alkyl ester, such as testosterone undecanoate). In some embodiments, steady state of a testosterone, testosterone alkyl ester (e.g., testosterone undecanoate), and dihydrotestosterone are obtained by the administration of about 0.1 mg to about 5 mg of a steroidal compound (e.g., a testosterone alkyl ester, such as testosterone undecanoate) per kg of an individual to whom the oral dosage form is to be administered. In certain embodiments, testosterone, testosterone alkyl ester (e.g., testosterone undecanoate), and dihydrotestosterone are obtained by the oral administration of about 1 mg to about 1 g, about 5 mg to about 500 mg, about 10 mg to about 300 mg, or about 20 to about 250 mg of a steroidal compound (e.g., a testosterone alkyl ester, such as testosterone undecanoate) to an individual upon once a day, twice a day, three times a day, or four times a day. In certain embodiments, the pharmacokinetic and/or pharmacodynamic profiles described herein are obtained as a function of dose of steroidal compound and/or formulation of the pharmaceutical composition. In certain embodiments, an oral dosage form for administration to a human female comprises about 10% as much of a testosterone alkyl ester as does an oral dosage form for administration to a human male. In some embodiments, a pharmaceutical composition for delivery to an adult human female comprises about 5 mg to about 50 mg, about 5 mg to about 30 mg, about 7 mg to about 15 mg, about 8 mg to about 14 mg, about 9 mg to about 14 mg, about 10 mg to about 13 mg, about 11 mg to about 13 mg, about 8 mg, or about 12 mg of a testosterone alkyl ester, such as testosterone undecanoate.

Provided in certain embodiments herein is a pharmaceutical composition or oral dosage form that provides or is formulated to provide a delayed release dosage form. In specific embodiments, any delayed release oral dosage form described herein comprises one or more steroidal compound (e.g., one or more testosterone alkyl ester, such as testosterone undecanoate). In certain embodiments, a delayed release dosage form is one that releases about 90% or less, about 80% or less, about 70% or less, about 60% or less, about 55% or less, about 50% or less, about 45% or less, about 40% or less, about 35% or less, about 5% to about 90%, about 5% to about 80%, about 5% to about 70%, about 5% to about 60%, about 5% to about 55%, about 5% to about 50%, about 5% to about 45%, about 5% to about 40%, about 5% to about 35%, about 20% to about 90%, about 20% to about 80%, about 20% to about 70%, about 20% to about 60%, about 20% to about 55%, about 20% to about 50%, about 20% to about 45%, about 20% to about 40%, or about 20% to about 35% of the steroidal compound (e.g., a testosterone alkyl ester, such as testosterone undecanoate) after 1 hour in an aqueous medium; releases about 90% or less, about 80% or less, about 70% or less, about 60% or less, about 50% or less, about 40% or less, about 30% or less, about 20% or less, about 2% to about 90%, about 2% to about 80%, about 2% to about 70%, about 2% to about 60%, about 2% to about 50%, about 2% to about 40%, about 2% to about 30%, about 2% to about 20%, about 10% to about 90%, about 10% to about 80%, about 10% to about 70%, about 10% to about 60%, about 10% to about 50%, about 10% to about 40%, about 10% to about 30%, or about 10% to about 20% of the steroidal compound (e.g., a testosterone alkyl ester, such as testosterone undecanoate) after 30 minutes in an aqueous medium; releases about 99% or less, about 98% or less, about 97% or less, about 96% or less, about 95% or less, about 90% or less, about 10% to about 99%, about 10% to about 98%, about 10% to about 97%, about 10% to about 96%, about 10% to about 95%, about 10% to about 90%, about 40% to about 99%, about 40% to about 98%, about 40% to about 97%, about 40% to about 96%, about 40% to about 95%, about 40% to about 90%, about 70% to about 99%, about 70% to about 98%, about 70% to about 97%, about 70% to about 96%, about 70% to about 95%, or about 70% to about 90% of the steroidal compound (e.g., a testosterone alkyl ester, such as testosterone undecanoate) after 3 hour in an aqueous medium; and/or releases more than 80% of the steroidal compound (e.g., a testosterone alkyl ester, such as testosterone undecanoate) contained therein within 12, 10, 8, 6, 5, 4, 3, or 2 hours in an aqueous medium. Conversely, in some embodiments an immediate release dosage form (e.g., a fast release dosage form) comprising a steroidal compound (e.g., a testosterone alkyl ester, such as testosterone undecanoate) releases about 90% or more of the steroidal compound (e.g., a testosterone alkyl ester, such as testosterone undecanoate) contained therein within about 15 minutes of exposure to an aqueous medium. In some instances, the aqueous medium is present in a USP Type-II (paddle) apparatus with conditions at 37±0.5° C. and at 100 rpm. In more specific instances, the aqueous medium is about 1 L of DI water having 8% w/v of Triton X-100. Furthermore, in some embodiments, an immediate release dosage form of steroidal compound (e.g., a testosterone alkyl ester, such as testosterone undecanoate) is an oral dosage form (e.g., capsule) comprising the steroidal compound (e.g., a testosterone alkyl ester, such as testosterone undecanoate) formulated in a mixture of castor oil and propylene glycol laurate (e.g., a composition comprising testosterone undecanoate, castor oil and propylene glycol laurate, as currently marketed under the tradename ANDRIOL); or the steroidal compound (e.g., a testosterone alkyl ester, such as testosterone undecanoate) formulated in oleic acid (e.g., a composition comprising testosterone undecanoate and oleic acid, as previously marketed under the tradename ANDRIOL).

Furthermore, provided herein is a delayed release oral dosage form formulated such that it provides, following a single oral administration, a mean plasma concentration of testosterone that is at least 50% lower, at least 40% lower, at least 30% lower, at least 20% lower, at least 10% lower, at least 5% lower, about 50-95% lower, about 40-95% lower, about 30-95% lower, about 20-95% lower, about 50-90% lower, about 40-80% lower, about 30-80% lower, about 20-80% lower, about 40-60% lower, or about 10-95% lower measured after about 1 hour than is provided by a single dose of an immediate release oral dosage form having the same amount of steroidal compound (e.g., a testosterone alkyl ester, such as testosterone undecanoate). In specific embodiments, provided herein is a delayed release oral dosage form formulated such that it provides, following a single oral administration, a mean plasma concentration of testosterone that is at least 20% lower measured after about 1 hour than is provided by a single dose of an immediate release oral dosage form. In some embodiments, provided herein is a delayed release oral dosage form formulated such that it provides, following a single oral administration, a mean plasma concentration of testosterone that is at least 50% lower, at least 40% lower, at least 30% lower, at least 20% lower, at least 10% lower, about 50-95% lower, about 40-95% lower, about 30-95% lower, about 20-95% lower, about 40-60% lower, about 20-80% lower, about 10-60% lower, or about 10-95% lower measured after about 2 hours than is provided by a single dose of an immediate release oral dosage form. In specific embodiments, provided herein is a delayed release oral dosage form formulated such that it provides, following a single oral administration, a mean plasma concentration of testosterone that is at least 20% lower measured after about 2 hours than is provided by a single dose of an immediate release oral dosage form. In certain embodiments, provided herein is a delayed release oral dosage form formulated such that it provides, following a single oral administration, a mean plasma concentration of testosterone that is at least 50% lower, at least 40% lower, at least 30% lower, at least 20% lower, at least 10% lower, about 50-95% lower, about 40-95% lower, about 30-95% lower, about 20-95% lower, about 50-80% lower, about 40-80% lower, about 30-60% lower, about 20-50% lower, about 10-50% lower, or about 10-95% lower measured after about 3 hours than is provided by a single dose of an immediate release oral dosage form. In specific embodiments, provided herein is a delayed release oral dosage form formulated such that it provides, following a single oral administration, a mean plasma concentration of testosterone that is at least 20% lower measured after about 3 hours than is provided by a single dose of an immediate release oral dosage form.

Provided in some embodiments herein is a delayed release oral dosage form formulated such that it provides, following a single oral administration, a mean plasma Cₘₐₓ of testosterone that is at least 25% lower, at least 20% lower, at least 15% lower, at least 10% lower, at least 5% lower, about 25-95% lower, about 20-99% lower, about 15-99% lower, about 10-99% lower, about 25-50% lower, about 20-60% lower, about 15-40% lower, about 10-60% lower, about 5-30% lower, or about 5-99% lower than the mean plasma Cₘₐₓ of testosterone that is provided by a single dose of an immediate release oral dosage form having an identical amount of the steroidal compound (e.g., a testosterone alkyl ester, such as testosterone undecanoate) as is present in the delayed release oral dosage form. In certain embodiments, provided herein is a delayed release oral dosage form that provides or is formulated to provide, following a single oral administration, a mean plasma Cₘₐₓ of the testosterone alkyl ester (e.g., testosterone undecanoate) that is at least 25% lower, at least 20% lower, at least 15% lower, at least 10% lower, at least 5% lower, about 25-95% lower, about 20-99% lower, about 15-99% lower, about 10-99% lower, about 5-99% lower, about 25-90% lower, about 20-80% lower, about 15-60% lower, about 10-60% lower, or about 5-40% lower than the mean plasma Cₘₐₓ of testosterone alkyl ester that is provided by a single dose of an immediate release oral dosage form having an identical amount of the steroidal compound (e.g., a testosterone alkyl ester, such as testosterone undecanoate) as is present in the delayed release oral dosage form. In some embodiments, provided herein is a delayed release oral dosage form that provides or is formulated to provide, following oral administration, a mean plasma Cₘₐₓ of dihydrotestosterone that is at least 10% lower, at least 8% lower, at least 7% lower, at least 6% lower, at least 5% lower, about 10-95% lower, about 8-99% lower, about 7-99% lower, about 6-99% lower, about 5-99% lower, about 5-15% lower, about 10-90% lower, about 8-80% lower, about 7-60% lower, about 10-60% lower, or about 5-40% lower than the mean plasma Cₘₐₓ of dihydrotestosterone provided by a single dose of an immediate release oral dosage form having an identical amount of the steroidal compound (e.g., a testosterone alkyl ester, such as testosterone undecanoate) as is present in the delayed release oral dosage form.

Provided in certain embodiments herein is a delayed release oral dosage form that provides or is formulated to provide a mean plasma Cₘₐₓ at steady state of testosterone alkyl ester that is at least 20% lower, at least 15% lower, at least 10% lower, at least 5% lower, about 20-95% lower, about 15-99% lower, about 10-99% lower, about 20-99% lower, about 15-99% lower, about 10-99% lower, about 5-99% lower, about 20-90% lower, about 20-80% lower, about 15-60% lower, about 10-60% lower, or about 5-40% lower than the mean plasma Cₘₐₓ of steroidal compound (e.g., a testosterone alkyl ester, such as testosterone undecanoate) at steady state provided by an immediate release oral dosage form having an identical amount of the testosterone alkyl ester as is present in the delayed release oral dosage form. In some embodiments, a delayed oral dosage form comprising testosterone alkyl ester provided herein provides or is formulated to provide a mean plasma Cₘₐₓ at steady state of testosterone that is at least 20% lower, at least 15% lower, at least 10% lower, at least 5% lower, about 20-95% lower, about 15-99% lower, about 10-99% lower, about 20-99% lower, about 15-99% lower, about 10-99% lower, about 10-30% lower, about 20-90% lower, about 20-80% lower, about 15-60% lower, about 10-60% lower, or about 10-40% lower than the mean plasma Cₘₐₓ of testosterone at steady state provided by an immediate release oral dosage form having an identical amount of the steroidal compound (e.g., a testosterone alkyl ester, such as testosterone undecanoate) as is present in the delayed release oral dosage form.

In some embodiments, the delayed release oral dosage form provides a fluctuation index of testosterone at steady state that is at least 20% lower, at least 15% lower, at least 10% lower, at least 5% lower, about 20-95% lower, about 15-99% lower, about 10-99% lower, about 20-99% lower, about 15-99% lower, about 10-99% lower, about 5-99% lower, about 20-90% lower, about 20-80% lower, about 15-60% lower, about 10-60% lower, or about 5-40% lower than a fluctuation index of testosterone at steady state of an immediate release oral dosage form having an identical amount of the steroidal compound (e.g., a testosterone alkyl ester, such as testosterone undecanoate) as is present in the delayed release oral dosage form. In certain embodiments, the delayed release oral dosage form provides a fluctuation index of testosterone alkyl ester at steady state that is at least 20% lower, at least 15% lower, at least 10% lower, at least 5% lower, about 20-95% lower, about 15-99% lower, about 10-99% lower, about 20-99% lower, about 15-99% lower, about 10-99% lower, about 5-99% lower, about 20-90% lower, about 20-80% lower, about 15-60% lower, about 10-60% lower, or about 5-40% lower than a fluctuation index of testosterone alkyl ester at steady state of an immediate release oral dosage form having an identical amount of the testosterone alkyl ester as is present in the delayed release oral dosage form. In some embodiments, a pharmaceutical composition or oral dosage form provided herein that does not comprise oleate provides a fluctuation index of testosterone at steady state that is at least 20% lower, at least 15% lower, at least 10% lower, at least 5%, about 20-95% lower, about 15-99% lower, about 10-99% lower, about 20-99% lower, about 15-99% lower, about 10-99% lower, about 5-99% lower, about 20-90% lower, about 20-80% lower, about 15-60% lower, about 10-60% lower, or about 5-40% lower than a fluctuation index of testosterone at steady state provided by an oleate-containing oral dosage form having an identical amount of the steroidal compound (e.g., a testosterone alkyl ester, such as testosterone undecanoate). In certain embodiments, a pharmaceutical composition or oral dosage form provided herein that does not contain castor oil (unmodified by polyoxylation or hydrogenation) provides a fluctuation index of testosterone alkyl ester at steady state that is at least 20% lower, at least 15% lower, at least 10% lower, at least 5% lower, about 20-95% lower, about 15-99% lower, about 10-99% lower, about 20-99% lower, about 15-99% lower, about 10-99% lower, about 5-99% lower, about 20-90% lower, about 20-80% lower, about 15-60% lower, about 10-60% lower, or about 5-40% lower than a fluctuation index of testosterone alkyl ester at steady state of an castor oil-containing oral dosage form having an identical amount of the steroidal compound (e.g., a testosterone alkyl ester, such as testosterone undecanoate). As utilized herein, the fluctuation index is the difference between the mean plasma Cₘₐₓ and mean plasma Cₘᵢₙ values that are achieved after administration of a dosage form.

In some embodiments, provided herein is a delayed oral dosage form that is formulated such that it provides, following a single oral administration, a mean plasma AUC_{0-∞} concentration of testosterone of that is at least 40%, at least 50% or at least 60% of the mean plasma AUC_{0-∞} concentration of testosterone provided by an immediate release dosage form.

### Methods of Treatment

In certain embodiments, provided herein are methods of treating an individual in need of an androgen therapy with any pharmaceutical composition or oral dosage form described herein. In some embodiments, provided are methods of treating androdeficiency in an individual in need thereof by administering to the individual any pharmaceutical composition or dosage form described herein, wherein the pharmaceutical composition or dosage form described herein comprises a therapeutically effective amount of a steroidal compound (e.g., one or more testosterone alkyl ester, such as testosterone undecanoate). In some embodiments, individuals are androdeficient (e.g., hypogonadal, andropausal, or otherwise androdeficient) adult male humans, young male humans who are suffering from delayed puberty (e.g., as a result of being hypogonadal), androdeficient (e.g., postmenopausal or otherwise androdeficient) adult female humans.

In specific embodiments, provided herein are methods of treating testosterone deficiency in male humans by administering to the male human any pharmaceutical composition or dosage form described herein, wherein the pharmaceutical composition or dosage form described herein comprises a therapeutically effective amount of one or more testosterone alkyl ester (e.g., testosterone undecanoate). In more specific embodiments, provided herein are methods of treating testosterone deficiency in hypogonadal male humans (e.g., adult or prepubescent male humans) by administering to the hypogonadal male human any pharmaceutical composition or dosage form described herein, wherein the pharmaceutical composition or dosage form described herein comprises a therapeutically effective amount of one or more testosterone alkyl ester (e.g., testosterone undecanoate). Symptoms of testosterone deficiency may include, by way of non-limiting example, one or more of depression, reduced libido, low energy, anemia, osteoporosis, debilitating muscle weakness, or the like. Therefore, in some embodiments, such symptoms, when caused by or suspected of being caused by andro- or testosterone deficiency, are also treated, either individually or collectively, by administering to a male human in need thereof a pharmaceutical composition or oral dosage form described herein. In some embodiments, provided herein are methods of treating testosterone deficiency in male humans by administering to the male human any pharmaceutical composition or dosage form described herein, wherein the pharmaceutical composition or dosage form described herein comprises a therapeutically effective amount of one or more testosterone alkyl ester (e.g., testosterone undecanoate) co-administered with a 5-alpha reductase enzyme inhibitor (e.g. dutasteride, finesteride, isotertinoin, gallic acid, L-lysine, epigallocatechin gallate, saw palmetto, phytosterol complex, beta sitosterol, green tea extract, polyphenols etc.). In more specific embodiments, the enzyme inhibitor can be co-administered as a separate composition or be a part of the same testosterone alkyl ester-containing composition.

In some embodiments, provided herein are methods of treating sexual dysfunction in an individual in need thereof by administering the individual any pharmaceutical composition or dosage form described herein, wherein the pharmaceutical composition or dosage form described herein comprises a therapeutically effective amount of a steroidal compound (e.g., one or more testosterone alkyl ester, such as testosterone undecanoate). In certain embodiments, the individual is a male adult human. In some embodiments, the individual is a female adult human.

In specific embodiments, provided herein are methods of treating andro-deficiency in female humans (e.g., adult female humans) by administering to the female human any pharmaceutical composition or dosage form described herein, wherein the pharmaceutical composition or dosage form described herein comprises a therapeutically effective amount of one or more testosterone alkyl ester (e.g., testosterone undecanoate). In some embodiments, provided herein are methods of maintaining muscle and/or bone mass in female humans (e.g., adult female humans) by administering to the female human any pharmaceutical composition or dosage form described herein, wherein the pharmaceutical composition or dosage form described herein comprises a therapeutically effective amount of one or more testosterone alkyl ester (e.g., testosterone undecanoate).

Provided in various embodiments of the methods described herein, administered are pharmaceutical compositions comprising therapeutically effective amounts of one or more steroidal compound (e.g., one or more testosterone alkyl ester, such as testosterone undecanoate). In some embodiments, a therapeutically effective amount is between about 1 mg and about 1 g, or about 10 mg to about 200 mg of one or more steroidal compound (e.g., one or more testosterone alkyl ester, such as testosterone undecanoate). In specific embodiments, a therapeutically effective amount is about 10 mg to about 50 mg, about 15 mg to about 40 mg, about 20 mg, to about 30 mg, or about 25 mg of one or more steroidal compound (e.g., one or more testosterone alkyl ester, such as testosterone undecanoate). In other embodiments, a therapeutically effective amount is about 70 mg to about 150 mg, about 80 mg to about 140 mg, about 90 mg to about 140 mg, about 100 mg to about 130 mg, about 110 mg to about 130 mg, or about 120 mg of one or more steroidal compound (e.g., one or more testosterone alkyl ester, such as testosterone undecanoate). In some embodiments, a therapeutically effective amount is about 0.1 mg to about 5 mg per kg of an individual to whom the oral dosage form is administered. In certain embodiments, a therapeutically effective amount is about 1 mg to about 1 g, about 5 mg to about 500 mg, about 10 mg to about 300 mg, or about 20 to about 250 mg of a steroidal compound (e.g., a testosterone alkyl ester, such as testosterone undecanoate) per day.

In certain embodiments, the methods described herein a plasma Cₘₐₓ of testosterone that is less than 1500 ng/dL, about 100 ng/dL to about 1500 ng/dL, or about 500 ng/dL to about 1500 ng/dL in at least 85% of a population of individuals (following administration of a single dose and/or in the steady state). In some embodiments the methods described herein provide a plasma Cₘₐₓ of testosterone that is less than 1800 ng/dL, about 100 ng/dL to about 1800 ng/dL, or about 500 ng/dL to about 1800 ng/dL in at least 95% of a population of individuals (following administration of a single dose and/or in the steady state). In some embodiments, the methods described herein provide a plasma Cₘₐₓ of testosterone that is less than 2500 ng/dL, or about 100 ng/dL to 2500 ng/dL in all individuals (following administration of a single dose and/or in the steady state). In certain embodiments, the methods described herein provide a plasma concentration of testosterone at steady state that is between about 200 ng/dL and 1300 ng/dL in at least 75%, at least 80%, at least 85%, at least 90%, at least 95%, or at least 99% of a population of individuals. In some embodiments, the methods described herein provide a plasma concentration of testosterone at steady state that is between about 200 ng/dL and 1100 ng/dL in at least 75%, at least 80%, at least 85%, at least 90%, at least 95%, or at least 99% of a population of individuals. In certain embodiments, the methods described herein provide a plasma concentration of testosterone at steady state that is between about 300 ng/dL and 1000 ng/dL in at least 50%, at least 60%, at least 70%, at least 75%, at least 80%, at least 85%, at least 90%, at least 95%, or at least 99% of a population of individuals. Similarly, in various embodiments, the methods described herein provide any of the pharmacokinetic or pharmacodynamic profiles described for the pharmaceutical compositions or dosage forms described herein.

In various embodiments, the pharmaceutical compositions or dosage forms described herein are administered orally. In some embodiments, pharmaceutical compositions described herein comprise or are divided into one or more oral dosage forms described herein. Thus, in some embodiments, methods described herein comprise and/or pharmacokinetic or pharmacodynamic profiles described herein are achieved by administration of a plurality of oral dosage forms simultaneously, sequentially or in a substantially simultaneous manner. Furthermore, administration of pharmaceutical compositions or oral dosage forms described herein is achieved in any therapeutically effective manner. In some embodiments, the pharmaceutical composition or oral dosage form is administered once a day, twice a day, three times a day, four times a day, or the like. In some embodiments, a pharmaceutical composition or oral dosage form described herein is administered in the fed state. In certain embodiments, a pharmaceutical composition or oral dosage form described herein is administered with a meal, within 30 minutes of a meal, within 1 hour of a meal, or within 2 hours of a meal. In more specific embodiments, a pharmaceutical composition or oral dosage form described herein is administered with a meal, within 30 minutes after a meal, within 1 hour after a meal, or within 2 hours after a meal. In some embodiments, provided herein is a reduced food effect pharmaceutical composition or dosage form, the pharmaceutical composition or dosage form comprising the components as set forth in any embodiment described herein. In some embodiments, the reduced food effect pharmaceutical composition or dosage form provides, when orally administered in the fasted state, a maximum plasma concentration (Cₘₐₓ) of testosterone that is at least 90%, at least 80%, at least 70%, .at least 60%, at least 50%, at least 40%, at least 30%, at least 20%, at least 15%, at least 10%, or at least 5% of the maximum plasma concentration (Cₘₐₓ) of testosterone provided when the same or identical pharmaceutical composition or dosage form is administered in the fed state. In certain embodiments, the reduced food effect pharmaceutical composition or dosage form provides, when orally administered in the fasted state, a maximum plasma concentration (Cₘₐₓ) of testosterone alkyl ester that is at least 90%, at least 80%, at least 70%, .at least 60%, at least 50%, at least 40%, at least 30%, at least 20%, at least 15%, at least 10%, or at least 5% of the maximum plasma concentration (Cₘₐₓ) of testosterone alkyl ester provided when the same or identical pharmaceutical composition or dosage form is administered in the fed state. In some embodiments, the reduced food effect pharmaceutical composition or dosage form provides, when orally administered in the fasted state, a maximum plasma concentration (Cₘₐₓ) of dihydrotestosterone that is at least 90%, at least 80%, at least 70%, .at least 60%, at least 50%, at least 40%, at least 30%, at least 20%, at least 15%, at least 10%, or at least 5% of the maximum plasma concentration (Cₘₐₓ) of dihydrotestosterone provided when the same or identical pharmaceutical composition or dosage form is administered in the fed state.

In certain embodiments, provided herein is a method of treating androgen deficiency in an individual, or a disorder associated therewith, the method comprising administering to an individual in need thereof a therapeutically effective amount of any composition described herein. In some embodiments, a composition administered according to a method described herein is formulated so as to provide any pharmacokinetic and/or pharmacodynamic effect described herein. In certain embodiments, methods provided herein comprise the administration of a sufficient amount of a composition described herein so as to provide any pharmacokinetic or pharmacodynamic effect described herein. In various embodiments, any protocol described herein for the administration of compositions is optionally utilized in any methods described herein.

### Carriers

Provided herein are pharmaceutical compositions comprising a steroidal compound (e.g., one or more testosterone alkyl ester, such as testosterone undecanoate) and at least one pharmaceutically acceptable carrier. In certain embodiments, the at least one pharmaceutically acceptable carrier comprises at least one hydrophilic carrier (e.g., hydrophilic surfactant or additive), at least one lipophilic carrier (e.g., lipophilic surfactant or additive), and/or at least one viscosity enhancer or solidifying agent. In specific embodiments, the at least one pharmaceutically acceptable carrier is a hydrophilic carrier. In more specific embodiments, the at least one pharmaceutically acceptable carrier comprises or further comprises a lipophilic carrier. In further embodiments, the at least one pharmaceutically acceptable carrier comprises at least one hydrophilic carrier, at least one lipidic and/or lipophilic carrier, and at least one viscosity enhancer or solidifying agent.

In certain embodiments, hydrophilic carriers include, by way of non-limiting example, a hydrophilic surfactant. In various instances, hydrophilic surfactants are used to provide any one or more of several advantageous characteristics to the compositions, including, by way of non-limiting example: increased solubility of the active ingredient in at least one of the fractions of the carrier that is a solid carrier; improved dissolution of the active ingredient; improved dispersion and/or dissolution of the lipidic carrier; improved solubilization of the active ingredient upon dissolution; enhanced absorption and/or bioavailability of the active ingredient, particularly a hydrophilic, hydrophobic, or lipophilic active ingredient; and improved stability, both physical and chemical, of the active ingredient. In various embodiments, the hydrophilic surfactant includes either a single hydrophilic surfactant or a mixture of hydrophilic surfactants. Hydrophilic surfactants also include both ionic or non-ionic surfactants.

In some embodiments, lipophilic carriers include or further include, by way of non-limiting example, one or more lipophilic surfactant, including one or more lipophilic surfactant, one or more mono-, di-, or triglyceride, or mixtures thereof. In various instances, lipophilic surfactants provide any one or more of the advantageous characteristics listed above for hydrophilic surfactants, and/or enhance the function of other (e.g., hydrophilic) surfactants present in the pharmaceutical composition.

The terms “hydrophilic” and “lipophilic” are relative terms. Hydrophilicity and/or lipophilicity are determined in any manner suitable. In one instances, an empirical parameter is used to characterize the relative hydrophilicity and lipophilicity of the carriers described herein. For example, in one manner, the hydrophilicity and/or lipophilicity non-ionic amphiphilic compounds is the hydrophilic-lipophilic balance (the “HLB” value). Carriers or surfactants with lower HLB values are more lipophilic, and have greater solubility in oils, whereas surfactants with higher HLB values are more hydrophilic, and have greater solubility in aqueous mediums. This measure is suitable for the surfactants described herein because, generally, surfactants are amphiphilic as they comprise both a polar moiety (e.g., a polar non-charged or charged moiety) and a lipophilic moiety (e.g., an aliphatic group).

Using HLB values as a rough guide, hydrophilic surfactants are generally considered to be those compounds having an HLB value greater than about 10, as well as non-ionic, anionic, cationic, or zwitterionic compounds for which the HLB scale is not generally applicable. Similarly, lipophilic surfactants are compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values are not always authoritative indicators of the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Thus, keeping these considerations involved, a person having ordinary skill in the art (PHOSITA) is able to utilize HLB values and the identity of a given product to determine surfactants for suitable lipophilicity and/or hydrophilicity for use in the pharmaceutical compositions described herein.

As used herein, useful surfactants include any surfactant that is pharmaceutically acceptable and is suitable for use in a pharmaceutical composition. Suitable surfactants include anionic, cationic, zwitterionic and non-ionic surfactants. Provided herein (e.g., in the Tables) are several general classes of surfactants. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the present inventors, is more reliable.

Surfactants described in the Tables are illustrative and are provided as non-limiting examples. For example, refined, distilled or fractionated surfactants, purified fractions thereof, or re-esterified fractions, are also within the scope of surfactants described herein, although they are not specifically listed in the Tables.

In some embodiments, surfactants described herein include polyoxylated fatty acids, such as polyethoxylated fatty acids (i.e., PEG-fatty acid esters). Provided in Table 1 is a list of illustrative and non-limiting examples of polyethoxylated fatty acid monoester surfactants.

TABLE 1
PEG-Fatty Acid Monoester Surfactants

| | COMMERCIAL | |
|---|---|---|
| COMPOUND | PRODUCT (Supplier) | HLB |
| PEG 4-100 | Crodet L series (Croda) | >9 |
| monolaurate | | |
| PEG 4-100 | Crodet O series (Croda) | >8 |
| monooleate | | |
| PEG 4-100 | Crodet S series (Croda), | >6 |
| monostearate | Myrj Series (Atlas/ICI) | |
| PEG 400 distearate | Cithrol 4DS series (Croda) | >10 |
| PEG 100, 200, 300 | Cithrol ML series (Croda) | >10 |
| monolaurate | | |
| PEG 100, 200, 300 | Cithrol MO series (Croda) | >10 |
| monooleate | | |
| PEG 400 dioleate | Cithrol 4DO series (Croda) | >10 |
| PEG 400-1000 | Cithrol MS series (Croda) | >10 |
| monostearate | | |
| PEG-1 stearate | Nikkol MYS-IEX (Nikko), | 2 |
| | Coster KI (Condea) | |
| PEG-2 stearate | Nikkol MYS-2 (Nikko) | 4 |
| PEG-2 oleate | NikkoI MYO-2 (Nikko) | 4.5 |
| PEG-4 laurate | Mapeg ® 200 ML (PPG), | 9.3 |
| | Kessco ® PEG 200ML | |
| | (Stepan), LIPOPEG 2L | |
| | (LIPO Chem.) | |
| PEG-4 oleate | Mapeg ® 200 MO (PPG), | 8.3 |
| | Kessco ® PEG200 MO (Stepan) | |
| PEG-4 stearate | Kessco ® PEG 200 MS (Stepan), | 6.5 |
| | Hodag 20 S (Calgene), Nikkol | |
| | MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), | 8.5 |
| | Kessco ® PEG 300 MO (Stepan), | |
| | Nikkol MYO-6 (Nikko), | |
| | Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), | 13 |
| | LIPOPEG 4DL(Lipo Chem.) | |
| PEG-8 oleate | Mapeg ® 400 MO (PPG), | 12 |
| | Emulgante A8 (Condea); | |
| | Kessco PEG 400 MO (Stepan) | |
| PEG-8 stearate | Mapeg ® 400 MS (PPG), Myrj 45 | 12 |
| PEG-9 oleate | Emulgante A9 (Condea) | >10 |
| PEG-9 stearate | Cremophor 59 (BASF) | >10 |
| PEG-10 laurate | Nikkol MYL-10 (Nikko), | 13 |
| | Lauridac 10 (Croda) | |
| PEG-10 oleate | Nikkol MYO-10 (Nikko) | 11 |
| PEG-10 stearate | Nikkol MYS-10 (Nikko), | 11 |
| | Coster K100 (Condea) | |
| PEG-12 laurate | Kessco ® PEG 600ML (Stepan) | 15 |
| PEG-12 oleate | Kessco ® PEG 600MO (Stepan) | 14 |
| PEG-12 ricinoleate | (CAS #9004-97-1) | >10 |
| PEG-12 stearate | Mapeg ® 600 MS (PPG), | 14 |
| | Kessco ® PEG 600MS (Stepan) | |
| PEG-15 stearate | Nikkol TMGS-15 (Nikko), | 14 |
| | Koster K15 (Condea) | |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), | 16 |
| | Kessco ® PEG 1000 MS (Stepan), | |
| | Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | > 10 |
| PEG-40 laurate | Crodet L40 (Croda) | 17.9 |
| PEG-40 oleate | Crodet 040 (Croda) | 17.4 |
| PEG-40 stearate | Myrj 52, Emerest ® 2715 (Henkel), | >10 |
| | Nikkol MYS-40 (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet 0-100 (Croda) | 18.8 |
| PEG-100 stearate | Myrj 59, Arlacel ® 165 (ICI) | 19 |
| PEG-200 oleate | Albunol 200 MO (Taiwan Surf.) | >10 |
| PEG-400 oleate | LACTOMUL (Henkel), | >10 |
| | Albunol 400 MO (Taiwan Surf.) | |
| PEG-600 oleate | Albunol 600 MO (Taiwan Surf) | >10 |

Furthermore, in some embodiments, surfactants described herein include, by way of non-limiting example, polyethylene glycol (PEG) fatty acid diesters. Illustrative and non-limiting examples of PEG-fatty acid diesters are shown in Table 2.

TABLE 2
PEG-Fatty Acid Diester Surfactants

| | COMMERCIAL | |
|---|---|---|
| COMPOUND | PRODUCT (Supplier) | HLB |
| PEG-4 dilaurate | Mapeg ® 200 DL (PPG), | 7 |
| | Kessco ® PEG 200 DL (Stepan), | 6 |
| | LIPOPEG 2-DL (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), | 11 |
| | Kessco ® PEG 400 DL (Stepan), | |
| | LIPOPEG 4 DL (Lipo Chem.) | |
| PEG-8 dioleate | Mapeg ® 400 DO (PPG), | 8.8 |
| | Kessco ® PEG 400 DO (Stepan), | |
| | LIPOPEG 4 DO(Lipo Chem.) | |
| PEG-8 distearate | Mapeg ® 400 DS (PPG), | 11 |
| | CDS 400 (Nikkol) | |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), | 10 |
| | Kessco ® 600 DO(Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | > 10 |

As discussed above, in some embodiments, pharmaceutical compositions described herein comprise mixtures of surfactants, including, e.g., mixtures of two or more commercial surfactant products. Several PEG-fatty acid esters are marketed commercially as mixtures or mono- and diesters. Illustrative and non-limiting examples of surfactant mixtures are shown in Table 3.

TABLE 3
PEG-Fatty Acid Mono-and Diester Mixtures

| | | |
|---|---|---|
| | COMMERCIAL | |
| COMPOUND | PRODUCT (Supplier) | HLB |

| | |
|---|---|
| PEG 4-150 mono, | Kessco ® PEG 200-6000 mono, dilaurate |
| dilaurate | (Stepan) |
| PEG 4-150 mono, | Kessco ® PEG 200-6000 mono, dioteate |
| dioleate | (Stepan) |
| PEG 4-150 mono, | Kessco ® 200-6000 mono, distearate |
| distearate | (Stepan) |

In some embodiments, surfactants described herein include, by way of non-limiting example, polyethylene glycol glycerol fatty acid esters (PEG glycerol fatty acid esters). Illustrative and non-limiting examples of PEG glycerol fatty acid esters are shown in Table 4.

TABLE 4
PEG Glycerol Fatty Acid Esters

| | COMMERCIAL | |
|---|---|---|
| COMPOUND | PRODUCT (Supplier) | HLB |
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), | 13 |
| | Aldo ® MS-20 KFG (Lonza) | |
| PEG-20 glyceryl oleate | Tagat ® ( (Goldschmidt) | >10 |
| PEG-30 glyceryl oleate | Tagat ® 02 (Goldschmidt) | >10 |

In certain embodiments, surfactants of different degrees of lipophilicity or hydrophilicity are prepared by reaction of alcohols or polyalcohols with a variety of natural and/or hydrogenated oils. In some embodiments, the oils used are castor oil or hydrogenated castor oil or an edible vegetable oil such as corn oil, olive oil, peanut oil, palm kernel oil, apricot kernel oil, or almond oil. In specific embodiments, alcohols include glycerol, propylene glycol, ethylene glycol, polyethylene glycol, sorbitol, and pentaerythritol. In certain embodiments, such surfactants are utilized in the pharmaceutical compositions described herein. Illustrative and non-limiting examples of surfactants of this class suitable for use in the pharmaceutical compositions described herein are shown in Table 5.

TABLE 5
Transesterification Products of Oils and Alcohols

| | COMMERCIAL | |
|---|---|---|
| COMPOUND | PRODUCT (Supplier) | HLB |
| PEG-3 castor oil | Nikkol CO-3 (Nikko) | 3 |
| PEG-5, 9, and 16 | ACCONON CA series (ABITEC) | 6-7 |
| castor oil | | |
| PEG-20 castor oil | Emalex C-20 (Nihon Emulsion), | 11 |
| | Nikkol CO-20 TX (Nikko) | |
| PEG-23 castor oil | Emulgante EL23 | >10 |
| PEG-30 castor oil | Emalex C-30 (Nihon Emulsion), | 11 |
| | Alkamuls ® EL 620 (Rhone- | |
| | Poulenc), Incrocas 30 (Croda) | |
| PEG-35 castor oil | Cremophor EL and EL-P (BASF), | |
| | Emulphor EL, Incrocas-35 | |
| | (Croda), Emulgin RO 35 (Henkel) | |
| PEG-38 castor oil | Emulgante EL 65 (Condea) | |
| PEG-40 castor oil | Emalex C-40 (Nihon Emulsion), | 13 |
| | Alkamuls ® EL 719 (Rhone- | |
| | Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated | Nikkol HCO-5 (Nikko) | 6 |
| castor oil | | |
| PEG-7 hydrogenated | Simusol ® 989 (Seppic), | 6 |
| castor oil | Cremophor WO7 (BASF) | |
| PEG-10 hydrogenated | Nikkol HCO-10 (Nikko) | 6.5 |
| castor oil | | |
| PEG-20 hydrogenated | Nikkol HCO-20 (Nikko) | 11 |
| castor oil | | |
| PEG-25 hydrogenated | Simulsol ® 1292 (Seppic), | 11 |
| castor oil | Cerex ELS 250 (Auschem SpA) | |
| PEG-30 hydrogenated | Nikkol HCO-30 (Nikko) | 11 |
| castor oil | | |
| PEG-40 hydrogenated | Cremophor RH 40 (BASF), | 13 |
| castor oil | Croduret (Croda), Emulgin HRE 40 | |
| | (Henkel) | |
| PEG-45 hydrogenated | Cerex ELS 450 (Auschem Spa) | 14 |
| castor oil | | |
| PEG-50 hydrogenated | Emalex HC-50 (Nihon Emulsion) | 14 |
| castor oil | | |
| PEG-60 hydrogenated | Nikkol HCO-60 (Nikko); | 15 |
| castor oil | Cremophor RH 60 (BASF) | |
| PEG-80 hydrogenated | Nikkol HCO-80 (Nikko) | 15 |
| castor oil | | |
| PEG-100 | Nikkol HCO-100 (Nikko) | 17 |
| hydrogenated | | |
| castor oil | | |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot | Labrafil ® M 1944 CS (Gattefosse) | 4 |
| kernel oil | | |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated | Labrafil ® M 2130 BS (Gattefosse) | 4 |
| palm | | |
| kernel oil | | |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn | Crovol M40 (Croda) | 10 |
| glycerides | Crovol A40 (Croda) | 10 |
| PEG-20 almond | | |
| glycerides | | |
| PEG-25 trioleate | TAGAT ® TO (Goldschmidt) | 11 |
| PEG-40 palm | Crovol PK-70 | >10 |
| kernel oil | | |
| PEG-60 corn | Crovol M70(Croda) | 15 |
| glycerides | | 15 |
| PEG-60 almond | Crovol A70 (Croda) | |
| glycerides | | |
| PEG-4 caprylic/capric | Labrafac ® Hydro (Gattefosse), | 4-5 |
| triglyceride | | |
| PEG-8 caprylic/capric | Labrasol (Gattefosse), | >10 |
| glycerides | Labrafac CM 10 (Gattefosse) | |
| PEG-6 caprylic/capric | SOFTIGEN ® 767 (Hüls), | 19 |
| glycerides | Glycerox 767 (Croda) | |
| Lauroyl macrogol-32 | GELUCIRE 44/14 (Gattefosse) | 14 |
| glyceride | | |
| Stearoyl macrogol | GELUCIRE 50/13 (Gattefosse) | 13 |
| glyceride | | |
| Mono, di, tri, tetra | SorbitoGlyceride (Gattefosse) | <10 |
| esters of | | |
| vegetable oils | | |
| and sorbitol | | |
| Pentaerythrityl | Crodamol PTIS (Croda) | <10 |
| tetraisostearate | | |
| Pentaerythrityl | Albunol DS (Taiwan Surf.) | <10 |
| distearate | | |
| Pentaerythrityl | Liponate PO-4 (Lipo Chem.) | <10 |
| tetraoleate | | |
| Pentaerythrityl | Liponate PS-4 (Lipo Chem.) | <10 |
| tetrastearate | | |
| Pentaerythrityl | Liponate PE-810 (Lipo Chem.), | <10 |
| tetracaprylate/ | Crodamol PTC (Croda) | |
| tetracaprate | | |
| Pentaerythrityl | Nikkol Pentarate 408 (Nikko) | |
| tetraoctanoate | | |

In some embodiments, surfactants utilized in the pharmaceutical compositions described herein include, by way of non-limiting example, polyglycerized fatty acids. Illustrative and non-limiting examples of suitable polyglyceryl esters are shown in Table 6.

TABLE 6
Polyglycerized Fatty Acids

| | COMMERCIAL | |
|---|---|---|
| COMPOUND | PRODUCT (Supplier) | HLB |
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 isostearate | Nikkol DGMIS (Nikko) | 5-7 |
| Polyglyceryl-3 oleate | Caprol ® 3G0 (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), | 9 |
| | Nikkol Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 ricinoleate | Nikkol Hexaglyn PR-15 (Nikko) | |
| Polyglyceryl-10 linoleate | Nikkol Decaglyn I-LN (Nikko) | 12 |
| Polyglyceryl-6 pentaoleate | Nikkol Hexaglyn S-O (Nikko) | <10 |
| Polyglyceryl-3 dioleate | Cremophor G032 (BASF) | <10 |
| Polyglyceryl-3 distearate | Cremophor GS32 (BASF) | <10 |
| Polyglyceryl-4 pentaoleate | Nikkol Tetraglyn 5-O (Nikko) | <10 |
| Polyglyceryl-6 dioleate | Caprol ® 6G20 | 8.5 |
| | (ABITEC); Hodag PGO-62 | |
| | (Calgene), PLUROL | |
| | OLEIQUE CC 497 (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 pentaoleate | Nikkol Decaglyn 5-O (Nikko) | 3.5 |
| Polyglyceryl-10 septaoleate | Nikkol Decagtyn 7-O (Nikko) | 3 |
| Polyglyceryl-10 tetraoleate | Caprol ® 10G40 (ABITEC); | 6.2 |
| | Hodag PGO-62 (CALGENE), | |
| | Drewpol 10-4-O (Stepan) | |
| Polyglyceryl-10 decaisostearate | Nikkol Decaglyn 10-IS (Nikko) | <10 |
| Polyglyceryl-10 decaoleate | Drewpol 10-10-O (Stepan), | 3.5 |
| | Caprol 10G100 (ABITEC), | |
| | Nikkol Decaglyn 10-O | |
| Polyglyceryl-10 mono, | Caprol ® PGE 860 (ABITEC) | 11 |
| dioleate | | |
| Polyglyceryl polyricinoleate | Polymuls (Henkel) | 3-20 |

In some embodiments, surfactants utilized in the pharmaceutical compositions described herein include, by way of non-limiting example esters of propylene glycol and fatty acids. Illustrative and non-limiting examples of surfactants of this class are given in Table 7.

TABLE 7
Propylene Glycol Fatty Acid Esters

| | COMMERCIAL | |
|---|---|---|
| COMPOUND | PRODUCT (Supplier) | HLB |
| Propylene glycol monocaprylate | Capryol ® 90 (Gattefosse), | <10 |
| | Nikkol Sefsol 218 (Nikko) | |
| Propylene glycol monolaurate | Lauroglycol 90 (Gattefosse), | <10 |
| | Lauroglycol FCC (Gattefosse) | |
| Propylene glycol oleate | Lutrol OP2000 (BASF) | <10 |
| Propylene glycol myristate | Mirpyl | <10 |
| Propylene glycol monostearate | ADM PGME-03 (ADM), | 3-4 |
| | LIPO PGMS (Lipo Chem.), | |
| | Aldo ® PGHMS (Lonza) | |
| Propylene glycol hydroxy stearate | | <10 |
| Propylene glycol ricinoleate | PROPYMULS (Henkel) | <10 |
| Propylene glycol isostearate | | <10 |
| Propylene glycol monooleate | Myverol ® P-06 (Eastman) | <10 |
| Propylene | glycol Captex ® | >6 |
| dicaprylate/dicaprate | 200 (ABITEC), | |
| | Miglyol ® 840 (Huls), | |
| | Neobee ® M-20 | |
| | (Stepan) | |
| Propylene glycol dioctanoate | Captex ® 800 (ABITEC) | |
| Propylene | glycol LABRAFAC | >6 |
| caprylate/caprate | PG (Gattefosse) | |
| Propylene glycol dilaurate | | >6 |
| Propylene glycol distearate | Kessco ® PGDS (Stepan) | >6 |
| Propylene glycol dicaprylate | Nikkol Sefsol 228 (Nikko) | >6 |
| Propylene glycol dicaprate | Nikkol PDD (Nikko) | >6 |

As discussed above, mixtures of surfactants are also used, in some embodiments, in the pharmaceutical compositions described herein. Mixtures of surfactants include, by way of non-limiting example, mixtures of propylene glycol fatty acid esters and glycerol fatty acid esters are suitable and are commercially available. Illustrative and non-limiting examples of such mixtures of surfactants include, by way of non-limiting example, those shown in Table 8.

TABLE 8
Glycerol/Propylene Glycol Fatty Acid Esters

| | COMMERCIAL | |
|---|---|---|
| COMPOUND | PRODUCT (Supplier) | HLB |
| Oleic | ATMOS 300, Arlacel ® 186 (ICI) | 3-4 |
| Stearic | ATMOS 150 | 3-4 |

In certain embodiments, an important class of surfactants includes the class of mono- and diglycerides. These surfactants are generally lipophilic. Illustrative and non-limiting examples of these surfactants are given in Table 9.

TABLE 9
Mono- and Diglyceride Surfactants

| | COMMERCIAL | |
|---|---|---|
| COMPOUND | PRODUCT (Supplier) | HLB |
| Monopalmitolein (C16:1) | (Larodan) | <10 |
| Monoelaidin (C18:1) | (Larodan) | <10 |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl | Nikkol MGM (Nikko) | 3-4 |
| monomyristate (C14) | | |
| Glyceryl monooleate | PECEOL (Gattefosse), | 3-4 |
| (C18:1) | Hodag GMO-D, | |
| | Nikkol MGO (Nikko) | |
| Glyceryl monooleate | RYLO series (Danisco), | 3-4 |
| | DIMODAN series (Danisco), | |
| | EMULDAN (Danisco), | |
| | ALDO ® MO FG (Lonza), | |
| | Kessco GMO (Stepan), | |
| | MONOMULS ® series (Henkel), | |
| | TEGIN O, DREWMULSE | |
| | GMO (Stepan), Atlas G-695 (ICI), | |
| | GMOrphic 80 (Eastman), | |
| | ADM DMG-40, 70, and 100 (ADM), | |
| | Myverol ® (Eastman) | |
| Glycerol monooleate/ | OLICINE (Gattefosse) | 3-4 |
| linoleate | | |
| Glycerol monolinoleate | Maisine (Gattefosse), | 3-4 |
| | Myverol ® 18-92, | |
| | Myverol ® 18-06 (Eastman) | |
| Glyceryl | Softigen ® 701 (Huls), | 6 |
| ricinoleate | HODAG GMR-D (Calgene), | |
| | ALDO ® MR (Lonza) | |
| Glyceryl | ALDO ® MLD (Lonza), | 6.8 |
| monolaurate | Hodag GML (Calgene) | |
| Glycerol monopalmitate | Emalex GMS-P (Nihon) | 4 |
| Glycerol | Capmul ® GMS. (ABITEC), | 5-9 |
| monostearate | Myvaplex (Eastman), | |
| | IMWITOR ® 191 | |
| | (Hüls), CUTINA GMS, | |
| | Aldo ® MS (Lonza), | |
| | Nikkol MGS series | |
| | (Nikko) | |
| Glyceryl | Capmul ® GMO-K (ABITEC) | <10 |
| mono-, dioleate | | |
| Glyceryl | CUTINA MD-A, ESTAGEL-G18 | <10 |
| palmitic/stearic | | |
| Glyceryl acetate | Lamegin ® EE (Grünau GmbH) | <10 |
| Glyceryl laurate | Inwitor ® 312 (Hüls), | 4 |
| | Monomuls ® 90-45 (Grünau | |
| | GmbH), Aldo ® | |
| | MLD (Lonza) | |
| Glyceryl citrate/ | Imwitor ® 375 (Hüls) | <10 |
| lactate/oleate/linoleate | | |
| Glyceryl caprylate | Imwitor ® 308 (Hüls), | 5-6 |
| | Capmul ® MCMC8 (ABITEC) | |
| Glyceryl | Capmul ® MCM (ABITEC) | 5-6 |
| caprylate/caprate | | |
| Caprylic acid mono, | Imwitor ® 988 (Hüls) | 5-6 |
| diglycerides | | |
| Caprylic/capric | Imwitor ® 742 (Hüls) | <10 |
| glycerides | | |
| Mono-and diacetylated | Myvacet ® 9-45, | 3.8-4 |
| monoglycerides | Myvacet ® 9-40, | |
| | Myvacet ® 9-08 (Eastman), | |
| | Lamegin ® (Grünau) | |
| Glyceryl monostearate | Aldo ® MS, Arlacel ® 129 (ICI), | 4.4 |
| | LIPO GMS (Lipo Chem.), Imwitor ® | |
| | 191 (Hüls), Myvaplex (Eastman) | |
| Lactic acid esters of | LAMEGIN GLP (Henkel) | <10 |
| mono, diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of | GELUCIRE 39/01 (Gattefosse), | 1 |
| fatty acids | GELUCIRE 43/01 (Gattefosse) | 6 |
| | GELUCIRE 37/06 (Gattefosse) | |
| Dipalmitolein (C16:1) | (Larodan) | |
| 1,2 and 1,3-diolein | (Larodan) | <10 |
| (C18:1) | | |
| Dielaidin (C18:1) | (Larodan) | <10 |
| Dilinolein (C18:2) | (Larodan) | <10 |

In some embodiments, surfactants utilized in the pharmaceutical compositions described herein include sterols and derivatives of sterols. In various embodiments, these surfactants are hydrophilic or lipophilic. Illustrative and non-limiting examples of surfactants of this class are shown in Table 10.

TABLE 10
Sterol and Sterol Derivative Surfactants

| | COMMERCIAL | |
|---|---|---|
| COMPOUND | PRODUCT (Supplier) | HLB |
| Cholesterol, sitosterol, | | <10 |
| lanosterol | | |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-S (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

In some embodiments, surfactants useful in the pharmaceutical compositions described herein include a variety of PEG-sorbitan fatty acid esters. In general, these surfactants are hydrophilic, although several lipophilic surfactants of this class can be used. Illustrative and non-limiting examples of these surfactants are shown in Table 11.

TABLE 11
PEG-Sorbitan Fatty Acid Esters

| | COMMERCIAL | |
|---|---|---|
| COMPOUND | PRODUCT (Supplier) | HLB |
| PEG-10 sorbitan laurate | Liposorb L-10 (Lipo Chem.) | >10 |
| PEG-20 sorbitan monolaurate | Tween-20 (Atlas/ICI), | 17 |
| | Crillet 1 (Croda), | |
| | DACOL MLS 20 (Condea) | |
| PEG-4 sorbitan monolaurate | Tween-21 (Atlas/ICI), | |
| | Crillet 11 (Croda) | |
| PEG-80 sorbitan monolaurate | Hodag PSML-80 (Calgene); | >10 |
| | T-Maz 28 | |
| PEG-6 sorbitan monolaurate | Nikkol GL-1 (Nikko) | 16 |
| PEG-20 sorbitan monopalmitate | Tween-40 (Atlas/ICI), | 16 |
| | Crillet 2 (Croda) | |
| PEG-20 sorbitan monostearate | Tween-60 (Atlas/ICI), | 15 |
| | Crillet 3 (Croda) | |
| PEG-4 sorbitan monostearate | Tween-61 (Atlas/ICI), | 9.6 |
| | Crillet 31 (Croda) | |
| PEG-8 sorbitan monostearate | DACOL MSS (Condea) | >10 |
| PBG-6 sorbitan monostearate | Nikkol TS106 (Nikko) | 11 |
| PEG-20 sorbitan tristearate | Tween-65 (Atlas/ICI), | 11 |
| | Crillet 35 (Croda) | |
| PEG-6 sorbitan tetrastearate | Nikkol GS-6 (Nikko) | 3 |
| PEG-60 sorbitan tetrastearate | Nikkol GS-460 (Nikko) | 13 |
| PEG-5 sorbitan monooleate | Tween-81 (Atlas/ICI), | 10 |
| | Crillet 41 (Croda) | |
| PEG-6 sorbitan monooleate | Nikkol TO-106 (Nikko) | 10 |
| PEG-20 sorbitan monooleate | Tween-80 (Atlas/ICI), | 15 |
| | Crillet 4 (Croda) | |
| PEG-40 sorbitan oleate | Emalex ET 8040 | 18 |
| | (Nihon Emulsion) | |
| PEG-20 sorbitan trioleate | Tween-85 (Atlas/ICI), | 11 |
| | Crillet 45 (Croda) | |
| PEG-6 sorbitan tetraoleate | Nikkol GO-4 (Nikko) | 8.5 |
| PEG-30 sorbitan tetraoleate | Nikkol GO-430 (Nikko) | 12 |
| PEG-40 sorbitan tetraoleate | Nikkol GO-440 (Nikko) | 13 |
| PEG-20 | sorbitan Tween-120 (Atlas/ICI), | >10 |
| monoisostearate | Crillet 6 (Croda) | |
| PEG sorbitol hexaoleate | Atlas G-1086 (ICI) | 10 |
| PEG-6 sorbitol hexastearate | Nikkol GS-6 (Nikko) | 3 |

In some embodiments, surfactants utilized herein include ethers of polyethylene glycol and alkyl alcohols. Illustrative and non-limiting examples of these surfactants are shown in Table 12.

TABLE 12
Polyethylene Glycol Alkyl Ethers

| | COMMERCIAL PRODUCT | |
|---|---|---|
| COMPOUND | (Supplier) | HLB |
| PEG-2 oleyl ether, oleth-2 | Brij 92/93 (Atlas/ICI) | 4.9 |
| PEG-3 oleyl ether, oleth-3 | Volpo 3 (Croda) | <10 |
| PEG-5 oleyl ether, oleth-5 | Volpo 5 (Croda) | <10 |
| PEG-10 oleyl ether, oleth-10 | Volpo 10 (Croda), | 12 |
| | Brij 96/97 (Atlas/ICI) | |
| PEG-20 oleyl ether, oleth-20 | Volpo 20 (Croda), | 15 |
| | Brij 98/99 (Atlas/ICI) | |
| PEG-4 lauryl ether, laureth-4 | Brij 30 (Atlas/ICI) | 9.7 |
| PEG-9 lauryl ether | | >10 |
| PEG-23 lauryl ether, laureth-23 | Brij 35 (Atlas/ICI) | 17 |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

In certain embodiments, surfactants utilized in the pharmaceutical compositions described herein include esters of sugars. Illustrative and non-limiting examples of such surfactants are shown in Table 13.

TABLE 13
Sugar Ester Surfactants

| COMPOUND | COMMERCIAL PRODUCT (Supplier) | HLB |
|---|---|---|
| Sucrose distearate | SUCRO ESTER 7 (Gattefosse), | 3 |
| | Crodesta F-10 (Croda) | |
| Sucrose distearate/ | SUCRO ESTER 11 (Gattefosse), | 12 |
| monostearate | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate | 15 |
| | 1695 (Mitsubishi-Kasei) | |

In some embodiments, surfactants utilized in the pharmaceutical compositions described herein include polyethylene glycol alkyl phenols, e.g., hydrophilic PEG-alkyl phenol surfactants. Illustrative and non-limiting examples of these surfactants are shown in Table 14.

TABLE 14
Polyethylene Glycol Alkyl Phenol Surfactants

| COMPOUND | COMMERCIAL PRODUCT (Supplier) | HLB |
|---|---|---|
| PEG-10-100 | Triton X series (Rohm & Haas), | >10 |
| nonyl phenol | Igepal CA series (GAF, USA), | |
| | Antarox CA series (GAF, UK) | |
| PEG-15-100 | Triton N-series (Rohm & Haas), | >10 |
| octyl phenol ether | Igepal CO series (GAF, USA), | |
| | Antarox CO series (GAF, UK) | |

In certain embodiments, surfactants utilized in pharmaceutical compositions described herein include polyoxyethylene-polyoxypropylene block copolymers. POE-POP block copolymers are a unique class of polymeric surfactants. The unique structure of the surfactants, with hydrophilic POE and lipophilic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. The generic term for these polymers is “poloxamer” (CAS 9003-11-6). These polymers have the formula: HO(C₂H₄O)ₐ(C₃H₆O)_{b}(C₂H₄O)ₐH; wherein the terms “a” and “b” denote the number of polyoxyethylene and polyoxypropylene units, respectively.

Illustrative and non-limiting examples of suitable surfactants of this class are shown in Table 15. Since the compounds are widely available, commercial sources are not listed in the Table. The compounds are listed by generic name, with the corresponding “a” and “b” values.

TABLE 15
POE-POP Block Copolymers

| | a, b values in | |
|---|---|---|
| COMPOUND | HO(C₂H₄O)ₐ(C₃H₆O)_{b}(C₂H₄O)ₐH | HLB |
| Poloxamer 105 | a = 11; b = 16 | 8 |
| Poloxamer 108 | a = 46; b = 16 | >10 |
| Poloxamer 122 | a = 5; b = 21 | 3 |
| Poloxamer 123 | a = 7; b = 21 | 7 |
| Poloxamer 124 | a = 11; b = 21 | >7 |
| Poloxamer 181 | a = 3; b = 30 | |
| Poloxamer 182 | a = 8; b = 30 | 2 |
| Poloxamer 183 | a = 10; b = 30 | |
| Poloxamer 184 | a = 13; b = 30 | |
| Poloxamer 185 | a = 19; b = 30 | |
| Poloxamer 188 | a = 75; b = 30 | 29 |
| Poloxamer 212 | a = 8; b = 35 | |
| Poloxamer 215 | a = 24; b = 35 | |
| Poloxamer 217 | a = 52; b = 35 | |
| Poloxamer 231 | a = 16; b = 39 | |
| Poloxamer 234 | a = 22; b = 39 | |
| Poloxamer 235 | a = 27; b = 39 | |
| Poloxamer 237 | a = 62; b = 39 | 24 |
| Poloxamer 238 | a = 97; b = 39 | |
| Poloxamer 282 | a = 10; b = 47 | |
| Poloxamer 284 | a = 21; b = 47 | |
| Poloxamer 288 | a = 122; b = 47 | >10 |
| Poloxamer 331 | a = 7; b = 54 | 0.5 |
| Poloxamer 333 | a = 20; b = 54 | |
| Poloxamer 334 | a = 31; b = 54 | |
| Poloxamer 335 | a = 38; b = 54 | |
| Poloxamer 338 | a = 128; b = 54 | |
| Poloxamer 401 | a = 6; b = 67 | |
| Poloxamer 402 | a = 13; b = 67 | |
| Poloxamer 403 | a = 21; b = 67 | |
| Poloxamer 407 | a = 98; b = 67 | |

In some embodiments, surfactants utilized in pharmaceutical compositions described herein include sorbitan esters of fatty acids. Illustrative and non-limiting examples of such surfactants are shown in Table 16.

TABLE 16
Sorbitan Fatty Acid Ester Surfactants

| COMPOUND | COMMERCIAL PRODUCT (Supplier) | HLB |
|---|---|---|
| Sorbitan monolaurate | Span-20 (Atlas/ICI), Crill 1 (Croda), | 8.6 |
| | Arlacel ® 20 (ICI) | |
| Sorbitan monopalmitate | Span-40 (Atlas/ICI), Crill 2 (Croda), | 6.7 |
| | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C ® (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan monoisostearate | Crill 6 (Croda), Nikkol SI-10 (Nikko) | 4.7 |
| Sorbitan sesquistearate | Nikkol SS-15 (Nikko) | 4.2 |

In certain embodiments, surfactants utilized in pharmaceutical compositions described herein include esters of lower alcohols (C₂ to C₄) and fatty acids (C₈ to C₁₈). Illustrative and non-limiting examples of these surfactants are shown in Table 17.

TABLE 17
Lower Alcohol Fatty Acid Ester Surfactants

| COMPOUND | COMMERCIAL PRODUCT (Supplier) | HLB |
|---|---|---|
| Ethyl oleate | Crodamol EO (Croda), | <10 |
| | Nikkol EOO (Nikko) | |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

In some embodiments, hydrophilic surfactants utilized in pharmaceutical compositions described herein include ionic surfactants (e.g., cationic, anionic and zwitterionic surfactants). In specific embodiments, anionic surfactants include fatty acid salts and bile acid salts. In certain specific embodiments, cationic surfactants include carnitines. In some specific embodiments, ionic surfactants include, by way of non-limiting example, sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Illustrative and non-limiting examples of such surfactants are shown in Table 18. For simplicity, exemplary counterions are shown in the entries in the Table. In various embodiments, such counterions are optionally substituted with any suitable counterion. For example, although the fatty acids are shown as sodium salts, other cation counterions are optionally used, such as alkali metal cations or ammonium. Unlike certain non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in the Table.

TABLE 18
Ionic Surfactants

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glyco cheno deoxycholate | |
| Sodium cholylsarcosinate | |
| Sodium N-methyl taurocholate | |
| Sodium lithocholate | |
| PHOSPHOLIPIDS | |
| Egg/Soy lecithin [Epikuron ™ (Lucas Meyer), | |
| Ovothin ™ (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol | |
| ethoxylates with phosphoric acid or anhydride | |
| CARBOXYLATES | |
| Ether carboxylates (by oxidation of terminal OH group of fatty | |
| alcohol ethoxylates) | |
| Succinylated monoglycerides [LAMEGIN ZE (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids | |
| calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA-sulfosuccinate | |
| CATIONIC Surfactants | >10 |
| Lauroyl carnitine | |
| Palmitoyl carnitine | |
| Myristoyl carnitine | |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine): | |
| Lauryl betaine (N-lauryl, N,N-dimethylglycine) | |
| Ethoxylated amines: | |
| Polyoxyethylene-15 coconut amine | |

In some embodiments, surfactants utilized in pharmaceutical compositions described herein include ionizable surfactants. In certain embodiments, ionizable surfactants, when present in their unionized (neutral, non-salt) form, are lipophilic surfactants suitable for use in the compositions of the present invention. Particular examples of such surfactants include free fatty acids, particularly C₆-C₂₂ fatty acids, and bile acids. More specifically, suitable unionized ionizable surfactants include the free fatty acid and bile acid forms of any of the fatty acid salts and bile salts shown in Table 18.

In some instances, derivatives of oil-soluble vitamins, such as vitamins A, D, E, K, etc., are also useful surfactants for use in the pharmaceutical compositions described herein. An example of such a derivative is tocopheryl PEG-1000 succinate (TPGS, available from Eastman).

In specific embodiments, surfactants or mixtures of surfactants that solidify (e.g., form a solid, a semi-solid, a gel, a jelly, a paste, or the like) at ambient room temperature are utilized in the pharmaceutical compositions described herein. In certain specific embodiments, surfactants or mixtures of surfactants utilized in the pharmaceutical compositions described herein solidify (e.g., form a solid, a semi-solid, a gel, a jelly, a paste, or the like) at ambient room temperature when combined with additional agents (e.g., particular lipophilic components, such as triglycerides, vitamins (e.g., Vitamin E), or the like, viscosity modifiers, stabilizers, solidifying agents, binders, thickeners, or the like). Such additional agents are optionally utilized in the pharmaceutical compositions described herein. In certain embodiments, pharmaceutical compositions described herein comprise a hydrophilic carrier (e.g., a hydrophilic surfactant), a lipophilic carrier, and/or a viscosity modifier or solidifying agent.

In some specific embodiments, non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; polyethoxylated fat-soluble vitamins or derivatives; and mixtures thereof.

In certain specific embodiments, the non-ionic hydrophilic surfactant is selected from, by way of non-limiting example, polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. In various embodiments, the glyceride is a monoglyceride, diglyceride, triglyceride, or a mixture thereof.

In some specific embodiments, non-ionic hydrophilic surfactants are the products of reaction mixtures of polyols and fatty acids, glycerides, vegetable oils, hydrogenated vegetable oils or sterols. These reaction mixtures are largely composed of the transesterification products of the reaction, along with often complex mixtures of other reaction products. In more specific embodiments, the polyol is glycerol, ethylene glycol, polyethylene glycol, sorbitol, propylene glycol, pentaerythritol, or a saccharide.

In certain specific embodiments, the hydrophilic surfactant is or includes an ionic surfactant. Specific ionic surfactants include alkyl ammonium salts; bile acids and salts, analogues, and derivatives thereof; fusidic acid and derivatives thereof; fatty acid derivatives of amino acids, oligopeptides, and polypeptides; glyceride derivatives of amino acids, oligopeptides, and polypeptides; acyl lactylates; mono-,diacetylated tartaric acid esters of mono-,diglycerides; succinylated monoglycerides; citric acid esters of mono-,diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

In some specific embodiments, ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono-,diacetylated tartaric acid esters of mono-,diglycerides; succinylated monoglycerides; citric acid esters of mono-diglycerides; carnitines; and mixtures thereof. In more specific embodiments, ionic surfactants include, by way of non-limiting example, lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof. In more specific embodiments, ionic surfactants are selected from lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

In various embodiments, lipophilic surfactants are selected from, by way of non-limiting example, alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils. As with the hydrophilic surfactants, lipophilic surfactants are optionally the products of reaction mixtures of polyols and fatty acids, glycerides, vegetable oils, hydrogenated vegetable oils, and sterols. In specific embodiments, lipophilic surfactants are selected from fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols. In certain specific embodiments, lipophilic surfactants are selected from lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof, with glycerol fatty acid esters and acetylated glycerol fatty acid esters being most preferred. Among the glycerol fatty acid esters, the esters are, e.g., mono- or diglycerides, or mixtures of mono- and diglycerides, where the fatty acid moiety is a C₆ to C₂₂fatty acid. In some specific embodiments, lipophilic surfactants are selected from the products of reaction mixture of polyols and fatty acids, glycerides, vegetable oils, hydrogenated vegetable oils, and sterols. In more specific embodiments, polyols are polyethylene glycol, sorbitol, propylene glycol, and pentaerythritol.

In certain embodiments, pharmaceutical compositions described herein include a lipophilic component or carrier. In some embodiments, the lipophilic carrier is selected from lipophilic surfactants, triglycerides, and Vitamin E compounds (e.g., dl-α-tocopherol). In specific embodiments, triglycerides utilized in the pharmaceutical compositions described herein are those that solidify (e.g., form a solid, a semi-solid, a gel, a jelly, a paste, or the like) at ambient room temperature, with or without addition of appropriate additives, or those which in combination with particular surfactants and/or active ingredients solidify at room temperature. Illustrative and non-limiting examples of triglycerides suitable for use in the pharmaceutical compositions described herein are shown in Table 19. In general, these triglycerides are readily available from commercial sources. For several triglycerides, representative commercial products and/or commercial suppliers are listed.

TABLE 19
Triglycerides

| Triglyceride | Commercial Source |
|---|---|
| Aceituno oil | |
| Almond oil | Super Refined Almond Oil (Croda) |
| Araehis oil | |
| Babassu oil | |
| Beeswax | |
| Blackcurrant seed oil | |
| Borage oil | |
| Buffalo ground oil | |
| Candlenut oil | |
| Canola oil | Lipex 108 (Abitec) |
| Castor oil | |
| Chinese vegetable tallow oil | |
| Cocoa butter | |
| Coconut oil | Pureco 76 (Abitec) |
| Coffee seed oil | |
| Corn oil | Super Refined Corn Oil (Croda) |
| Cottonseed oil | Super Refined Cottonseed Oil |
| | (Croda) |
| Crambe oil | |
| Cuphea species oil | |
| Evening primrose oil | |
| Grapeseed oil | |
| Groundnut oil | |
| Hemp seed oil | |
| Illipe butter | |
| Kapok seed oil | |
| Linseed oil | |
| Menhaden oil | Super Refined Menhaden Oil |
| | (Croda) |
| Mowrah butter | |
| Mustard seed oil | |
| Oiticica oil | |
| Olive oil | Super Refined Olive Oil (Croda) |
| Palm oil | |
| Palm kernel oil | |
| Peanut oil | Super Refined Peanut Oil (Croda) |
| Poppy seed oil | |
| Rapeseed oil | |
| Rice bran oil | |
| Safflower oil | Super Refined Safflower Oil |
| | (Croda) |
| Sal fat | |
| Sesame oil | Super Refined Sesame Oil (Croda) |
| Shark liver oil | Super Refined Shark Liver Oil |
| | (Croda) |
| Shea nut oil | |
| Soybean oil | Super Refined Soybean Oil |
| | (Croda) |
| Stillingia oil | |
| Sunflower oil | |
| Tall oil | |
| Tea seed oil | |
| Tobacco seed oil | |
| Tung oil (China wood oil) | |
| Ucuhuba | |
| Vernonia oil | |
| Wheat germ oil | Super Refined Wheat Germ Oil |
| | (Croda) |
| Hydrogenated castor oil | Castorwax |
| Hydrogenated coconut oil | Pureco 100 (Abitec) |
| Hydrogenated cottonseed oil | Dritex C (Abitec) |
| Hydrogenated palm oil | Dritex PST (Abitec); Softisan 154 |
| | (Hüls) |
| Hydrogenated soybean oil | Sterotex HM NF (Abitec); Dritex S |
| | (Abitec) |
| Hydrogenated vegetable oil | Sterotex NF (Abitec); |
| | Hydrokote M (Abitec) |
| Hydrogenated cottonseed and castor | Sterotex K (Abitec) |
| oil | |
| Partially hydrogenated soybean oil | Hydrokote AP5 (Abitec) |
| Partially hydrogenated soy and | Apex B (Abitec) |
| cottonseed oil | |
| Glyceryl mono-, di-, tri-behenate | Compritol 888 |
| Glycerol tributyrate | (Sigma) |
| Glyceryl tricaproate | (Sigma) |
| Glyceryl tricaprylate | (Sigma) |
| Glyceryl tricaprate | Captex ® 1000 (Abitec) |
| Glyceryl triundecanoate | Captex ® 8227 (Abitec) |
| Glyceryl trilaurate | (Sigma) |
| Glyceryl trimyristate | Dynasan 114 (Hüls) |
| Glyceryl tripalmitate | Dynasan 116 (Hüls) |
| Glyceryl tristearate | Dynasan 118 (Hüls) |
| Glyceryl triarchidate | (Sigma) |
| Glyceryl trimyristoleate | (Sigma) |
| Glyceryl tripalmitoleate | (Sigma) |
| Glyceryl trioleate | (Sigma) |
| Glyceryl trilinoleate | (Sigma) |
| Glyceryl trilinolenate | (Sigma) |
| Glyceryl tricaprylate/caprate | Captex ® 300 (Abitec); Captex ® 355 |
| | (Abitec); Miglyol 810 (Hüls); |
| | Miglyol 812 (Hüls) |
| Glyceryl tricaprylate/caprate/laurate | Captex ® 350 (Abitec) |
| Glyceryl tricaprylate/caprate/linoleate | Captex ® 810 (Abitec); |
| | Miglyol 818 (Hüls) |
| Glyceryl tricaprylate/caprate/stearate | Softisan 378 (Hüls); (Larodan) |
| Glyceryl tricaprylate/laurate/stearate | (Larodan) |
| Glyceryl 1,2-caprylate-3-linoleate | (Larodan) |
| Glyceryl 1,2-caprate-3-stearate | (Larodan) |
| Glyceryl 1,2-laurate-3-myristate | (Larodan) |
| Glyceryl 1,2-myristate-3-laurate | (Larodan) |
| Glyceryl 1,3-palmitate-2-butyrate | (Larodan) |
| Glyceryl 1,3-stearate-2-caprate | (Larodan) |
| Glyceryl 1,2-linoleate-3-caprylate | (Larodan) |

In certain embodiments, the triglycerides utilized in the pharmaceutical compositions described herein include fractionated triglycerides, modified triglycerides, synthetic triglycerides, and mixtures of triglycerides are also within the scope of the invention. In specific embodiments, triglycerides include, by way of non-limiting example, vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants”, may be suitable to provide all or part of the triglyceride component for the compositions of the present invention. Examples of commercial surfactant compositions containing triglycerides include some members of the surfactant families Gelucires (Gattefosse), Maisines (Gattefosse), and Imwitors (Hills). Specific examples of these compositions are: Gelucire 44/14 (saturated polyglycolized glycerides); Gelucire 50/13 (saturated polyglycolized glycerides); Gelucire 53/10 (saturated polyglycolized glycerides); Gelucire 33/01 (semi-synthetic triglycerides of C₈-C₁₈ saturated fatty acids); Gelucire 39/01 (semi-synthetic glycerides); other Gelucires, such as 37/06, 43/01, 35/10, 37/02, 46/07, 48/09, 50/02, 62/05, or the like; Maisine 35-I (linoleic glycerides); and Imwitor 742 (capiylic/capric glycerides).

### Additional Agents

The pharmaceutical compositions described herein optionally include one or more additional agents or additives. In certain instances, suitable additives include those that facilitate formulating a pharmaceutical composition described herein as an oral dosage form and include, e.g., coatings and capsule components. Further additives include, by way of non-limiting example, solubilizers, enzyme inhibitors, anti-foaming agents, antioxidants, binders, buffering agents, chelating agents, diluents, disintegrants, flavoring agents, preservatives, sweeteners, thickeners, or the like.

In some embodiments, pharmaceutical compositions provided herein optionally include one or more solubilizers, i.e., additives to increase the solubility of the pharmaceutical active ingredient or other composition components in the solid carrier. Suitable solubilizers for use in the compositions of the present invention include: alcohols, polyols, ethers of polyethylene glycols, amides, esters or the like. Alcohols and polyols include, by way of non-limiting example, ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives. Ethers of polyethylene glycols include those having an average molecular weight of about 200 to about 6000, such as, by way of non-limiting example, tetrahydrofurfuryl alcohol PEG ether (glycofurol, available commercially from BASF under the trade name Tetraglycol) and methoxy PEG (Union Carbide). Amides include, by way of non-limiting example, 2-pyrrolidone, 2-piperidone, F-caprolactam, N-alkylpyrrolidone, N-hydroxyalkylpyrrolidone, N-alkylpiperidone, N-alkylcaprolactam, dimethylacetamide, and polyvinylpyrrolidone. Esters include, by way of non-limiting example, ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof. Other solubilizers include, by way of non-limiting example, dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water. Mixtures of solubilizers are also within the scope of the present disclosure. Except as indicated, these compounds are readily available from standard commercial sources. In specific embodiments, solubilizers include, by way of non-limiting example, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. In certain specific embodiments, solubilizers include sorbitol, glycerol, triacetin, ethyl alcohol, PEG-400, glycofurol and propylene glycol. The amount of solubilizer included in the pharmaceutical compositions described herein is any suitable amount.

Anti-adherents (anti-sticking agents, glidants, flow promoters, lubricants) include, by way of non-limiting example, talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate. Antioxidants include, by way of non-limiting example, BHT, BHA, gallic acid, propyl gallate, ascorbic acid, ascorbyl palmitate, 4-hydroxymethyl-2,6-di-tert-butyl phenol, and tocopherol. Binders (adhesives), i.e., agents that impart cohesive properties to powdered materials through particle-particle bonding, include, by way of non-limiting example, matrix binders (dry starch, dry sugars), film binders (PVP, starch paste, celluloses, bentonite, sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose). Buffering agents, include an acid and a base, wherein the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid. Chelating agents include, by way of non-limiting example, EDTA and EDTA salts. Colorants or opaquants include, by way of non-limiting example, titanium dioxide, food dyes, lakes, natural vegetable colorants, iron oxides, silicates, sulfates, magnesium hydroxide and aluminum hydroxide. Diluents or fillers include, by way of non-limiting example, lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose. Disintegrants and super disintegrants include, by way of non-limiting example, croscarmellose sodium, starch, starch derivatives, clays, gums, cellulose, cellulose derivatives, alginates, crosslinked polyvinypyrrolidone, sodium starch glycolate and microcrystalline cellulose. Flavorants or desensitizers include, by way of non-limiting example, spray-dried flavors, essential oils and ethyl vanillin. Plasticizers include, by way of non-limiting example, polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate. Preservatives include, by way of non-limiting example, ascorbic acid, boric acid, sorbic acid, benzoic acid, and salts thereof, parabens, phenols, benzyl alcohol, and quaternary ammonium compounds. Solvents include, by way of non-limiting example, alcohols, ketones, esters, chlorinated hydrocarbons and water. Sweeteners include, by way of non-limiting example, natural sweeteners such as maltose, sucrose, glucose, sorbitol, glycerin and dextrins, and artificial sweeteners, such as aspartame, saccharine and saccharine salts. Thickeners (viscosity modifiers, thickening agents) include, by way of non-limiting example, sugars, polyvinylpyrrolidone, cellulosics, polymers, high molecular weight polyethylene glycols (e.g., PEG 8000), and alginates. Additives also include, by way of non-limiting example, proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate trimellitate, carboxymethylethyl cellulose, hydroxypropylmethyl cellulose phthalate); inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives can be readily determined by one skilled in the art, according to the particular properties desired.

### Dosage Forms

In various embodiments, pharmaceutical compositions described herein are formulated as oral dosage forms. Oral dosage forms are prepared by any suitable process including one or more steps of, by way of non-limiting example, agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, solvent dehydration, sonication, spheronization, spray chilling, spray congealing, spray drying, or the like.

In some embodiments, a pharmaceutical composition described herein is formulated with a substrate to form an oral dosage form. In various embodiments, substrates useful for formulating pharmaceutical compositions described herein as oral dosage forms include or comprise, by way of non-limiting example, a powder or a multiparticulate (e.g., one or more granule, one or more pellet, one or more bead, one or more spherule, one or more beadlet, one or more microcapsule, one or more millisphere, one or more mini capsule, one or more microcapsule, one or more nanocapsule, one or more nanosphere, one or more microsphere, one or more minitablet, one or more tablet, one or more capsule, or one or more combinations thereof). In certain instances, a powder constitutes a finely divided (milled, micronized, nanosized, precipitated) form of an active ingredient or additive molecular aggregates or a compound aggregate of multiple components or a physical mixture of aggregates of an active ingredient and/or additives.

Substrates are prepared from any suitable material including, by way of non-limiting example, sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin. Furthermore, the substrate is optionally composed of active ingredients, surfactants, triglycerides or additives described herein. In one particular embodiment, the substrate is a solid form of an additive, an active ingredient, a surfactant, or a triglyceride; a complex of an additive, surfactant or triglyceride and an active ingredient; a coprecipitate of an additive, surfactant or triglyceride and an active ingredient, or a mixture thereof.

In various embodiments, pharmaceutical compositions and substrates described herein provide or are formulated to provide an oral dosage from selected from, by way of non-limiting example, a minicapsule, a capsule, a tablet, an implant, a troche, a lozenge (minitablet), a temporary or permanent suspension, a wafer, a chewable tablet, a quick or fast dissolving tablet, an effervescent tablet, a buccal or sublingual solid, a granule, a film, a sprinkle, a pellet, a bead, a pill, a powder, a triturate, a strip or a sachet.

In specific embodiments, the oral dosage form described herein is a capsule. Suitable capsule forms include, by way of non-limiting example, hard or soft gelatin capsules, starch capsules, and cellulosic capsules. In more specific embodiments, oral dosage forms described herein are in the form of hard or soft gelatin capsules. In some embodiments, the oral dosage form is a capsule comprising a jelly, solid, semi-solid, glassy or paste-like composition, wherein the testosterone alkyl ester is formulated into the composition.

In specific embodiments, a pharmaceutical composition described herein is formulated as an oral dosage form by (i) heating a pharmaceutical compositions described herein until pharmaceutical composition has an ability to flow (e.g., it is a homogeneous solution, an emulsion, a slurry or the like); and (ii) depositing the pharmaceutical composition with an ability to flow on a substrate. In more specific embodiments, the pharmaceutical composition that has an ability to flow is a homogeneous solution. In further or alternative embodiments, the substrate is one or more capsule, one or more microcapsule, or one or more nanocapsule. In more specific embodiments, the substrate is a hard gelatin capsule or a soft gelatin capsule. In still more specific embodiments, the substrate is a hard gelatin capsule.

### EXAMPLES

### Example 1

In certain instances, oral dosage forms are prepared in the following manner:
Step 1: transfer the selected amounts of carriers and additives into a clean container and heat the combination until a molten solution is obtained;
Step 2: transfer the selected amount of steroidal compound (e.g., testosterone undecanoate) to the molten solution obtained in Step 1 and homogenize;
Step 3: maintain the mixture of Step 2 at an elevated temperature until used in Step 4; and
Step 4: encapsulation of the mixture of Step 3 (e.g., in a hard gelatin capsule).

Using the preceding process, the following capsules are prepared:

TABLE 20
Capsule 1

| Component | % w/w |
|---|---|
| Testosterone undecanoate | 15 |
| Polyoxy1 40 Hydrogenated Castor Oil, NF | 16 |
| Glyceryl Monolinoleate, NF (Maisine 35-1) | 63 |
| Polyethylene Glycol 8000, USP | 6 |
| Total | 100 |

TABLE 21
Capsule 2

| Component | % w/w |
|---|---|
| Testosterone undecanoate | 25 |
| Polyoxyl 35 Castor Oil, NF | 21 |
| Vitamin E, USP (d,l-α-tocopherol) | 48 |
| Polyethylene Glycol 8000, USP | 6 |
| Total | 100 |

TABLE 22
Capsule 3

| Component | % w/w |
|---|---|
| Testosterone undecanoate | 22 |
| Vitamin E Polyethylene Glycol Succinate, NF | 22 |
| Vitamin E, USP (d,l-tocopherol) | 34 |
| Polyethylene Glycol 8000, USP | 4 |
| Hypromellose (100 cP, K100 Premium LV) | 18 |
| Total | 100 |

TABLE 23
Capsule 4

| Component | % w/w |
|---|---|
| Testosterone undecanoate | 22 |
| Vitamin E Polyethylene Glycol Succinate, NF | 22 |
| Vitamin E, USP (d,l-tocopherol) | 34 |
| Polyethylene Glycol 8000, USP | 4 |
| Hypromellose (4,000 cP, K4M) | 18 |
| Total | 100 |

### Example 2

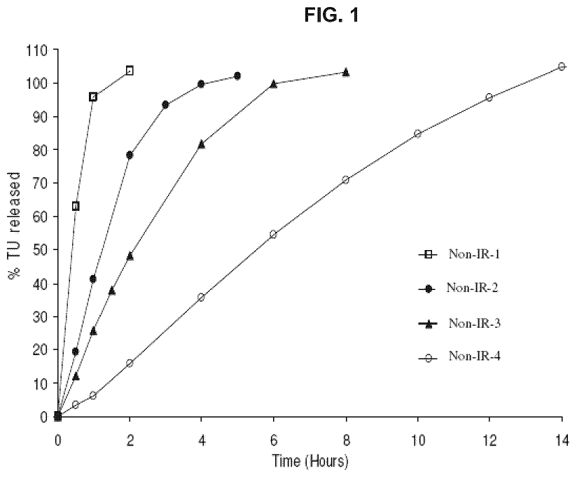

Capsules 1-4 are subjected to USP Type-II (paddle) apparatus conditions at 37±0.5° C., at 100 rpm (i.e., deposited in 1 L of DI water having 8% w/v of Triton X-100). FIG. 1 illustrates the release profiles of Capsules 1-4.

### Example 3

### Clinical Trial Protocol

Study Population: Healthy volunteers (N=24) with a BMI of 18-30 kg/m² and having a pre-trial screening total T concentration of less than 1.3 ng/mL (4.5 nmol/L). Healthy volunteers include post-menopausal women aged 45 or greater.

Study Design: Phase-I, single center, randomized, open-label, study of Capsules 1-4 and 3× an immediate release oral dosage form comprising 40 mg of testosterone undecanoate (so as to provide the same 120 mg dose as Capsules 1-4) formulated in a mixture of castor oil and propylene glycol laurate available under the tradename ANDRIOL.

Mode of administration: Orally with 240 mL of water about 30 minutes after starting a standardized, high fat, high calorie breakfast preceded by a 10 hour fast. Duration between treatments: minimum of 7 days between the start of each treatment period.

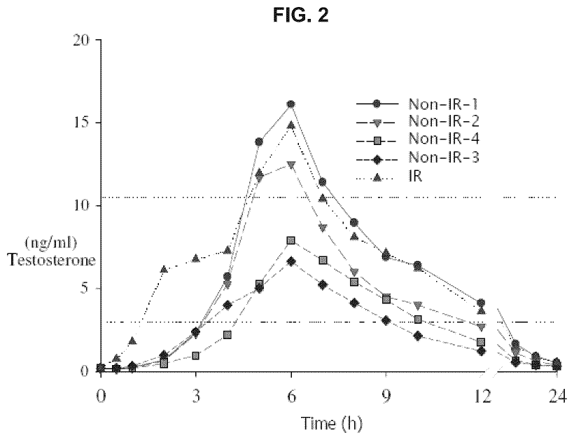

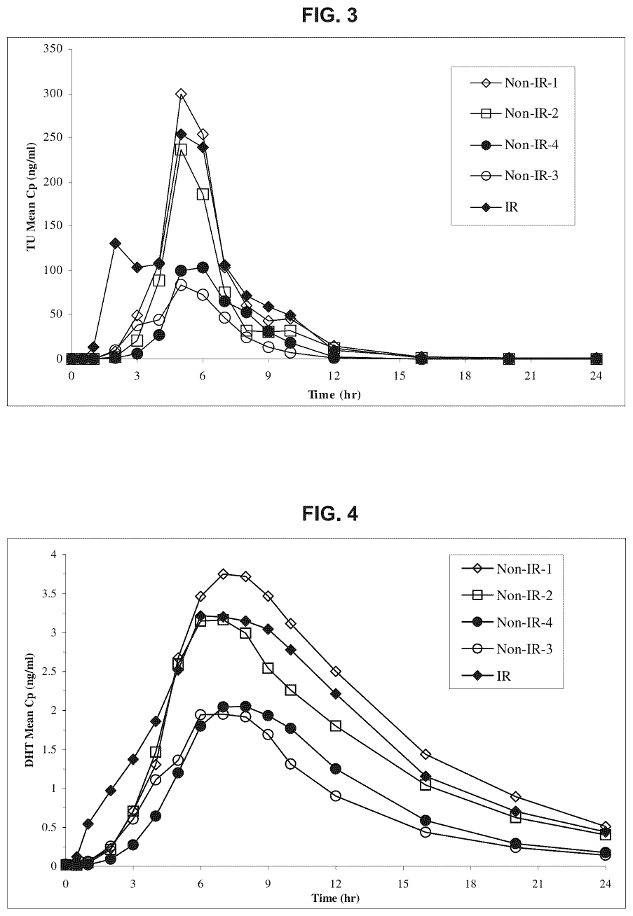

FIG. 2 illustrates the mean plasma testosterone concentrations following administration of Capsules 1-4 and 3× a 40 mg immediate release oral dosage form (for a total of a 120 mg immediate release dose). FIG. 3 illustrates the mean plasma testosterone undecanoate concentrations following administration of Capsules 1-4 and 3× a 40 mg immediate release oral dosage form (for a total of a 120 mg immediate release dose). FIG. 4 illustrates the mean plasma dihydrotestosterone concentrations following administration of Capsules 1-4 and 3× a 40 mg immediate release oral dosage form (for a total of a 120 mg immediate release dose).

Tables 24-26 illustrate the concentration levels of single administration and simulated steady state levels of testosterone, testosterone undecanoate, and dihydrotestosterone obtained.

TABLE 24
Mean Plasma Testosterone Levels

| | Capsule 1 | Capsule 2 | Capsule 3 | Capsule 4 | IR |
|---|---|---|---|---|---|
| Mean Cₘₐₓ (ng/mL) single dose | 18.2 | 13.6 | 7.5 | 8.8 | 19.7 |
| Mean Cₘₐₓ (ng/mL) steady state | 17.0 | 13.1 | 6.7 | 8.6 | 15.7 |
| Mean Cₘᵢₙ (ng/mL) steady state | 0.35 | 2.3 | 1.0 | 1.4 | 3.4 |

TABLE 25
Mean Plasma Testosterone Undecanoate Levels

| | Capsule 1 | Capsule 2 | Capsule 3 | Capsule 4 | IR |
|---|---|---|---|---|---|
| Mean Cₘₐₓ (ng/mL) single dose | 384.8 | 264.0 | 126.9 | 156.1 | 407.8 |
| Mean Cₘₐₓ (ng/mL) steady state | 260.1 | 187.1 | 78.7 | 111.5 | 240.8 |
| Mean Cₘᵢₙ (ng/mL) steady state | 15.0 | 8.9 | 1.1 | 1.8 | 9.6 |

TABLE 26
Mean Plasma Dihydrotestosterone Levels

| | Capsule 1 | Capsule 2 | Capsule 3 | Capsule 4 | IR |
|---|---|---|---|---|---|
| Mean Cₘₐₓ (ng/mL) single dose | 4.4 | 3.6 | 2.3 | 2.4 | 3.9 |
| Mean Cₘₐₓ (ng/mL) steady state | 4.6 | 4.0 | 2.3 | 2.3 | 4.1 |
| Mean Cₘᵢₙ (ng/mL) steady state | 2.2 | 1.6 | 0.9 | 1.0 | 2.2 |

### Methods of Preparation

### Testosterone Undecanoate

Commercially available testosterone undecanoate (TU) dosage forms such as, Andriol Testocaps, Jatenzo, Kyzatrex, are available as a one-piece soft gelatin capsule (softgel) dosage form with fully solubilized TU in liquid compositions filled in the capsule. These liquid filled softgel compositions, despite faster release of TU from the dosage form in a typical release media resulted in suboptimal testosterone replacement efficiency with shelf life/storage limitations, an important factor for inventory management. For the purposes of this application, shelf life shall be understood to be a period of (storage) time during which the pharmaceutical or drug product is considered fit for use and effective.

Manufacturing of a non-solid or liquid comprising composition in a hard gelatin capsule dosage form is less labor and time intensive relative to a softgel capsule dosage form, and often preferred. Moreover, for relatively water insoluble drug such as TU, a fully solubilized compositions in a softgel capsule are prone to precipitation or crusting of the TU during processing probably due to higher pre-drying gelatin ribbon water content, thereby, limiting the TU loading strength of a fully solubilized composition. Furthermore, often desired for variety of reasons, a semi-solid, a gel, a jelly, a paste, or the like forming compositions at room temperature may require higher than typical filling temperature utilized for filling into a softgel to prevent manufacturing disruption from clogging of the machinery and increased downtime. Due to filling temperature limitations softgel are not suitable for high temperature filling necessary for compositions that are a semi-solid, a gel, a jelly, a paste, or the like or non-liquid at room temperature. Compositions that are liquid at higher temperature but transform into a semi-solid, a gel, a jelly, a paste, or the like at room temperature rendering often better chemical stability, but slower release with possibly lower oral absorption/bioavailability relative to fully solubilized liquid compositions.

A non-solid (e.g. liquid, a semi-solid, a gel, a jelly, a paste, or the like) filled hard gelatin capsule is a type of oral dosage form used to deliver medications that are not easily administered in other forms. The capsule consists of two halves made of hard gelatin that are filled with a liquid medication and then sealed. While the pre-fabricated (off-the-shelf) hard gelatin capsule offers several advantages, it presents challenges with regards to fill volume, acceptable physical (no leaking) and chemical stability in conjunction with superior bioperformance.

There remains an unmet need for a testosterone undecanoate oral dosage form product that is palatable, easy to swallow, low resource intensive, easy to manufacture, of adequate oral bioavailability and bioperformance, and of acceptable or enhanced stability/extended shelf life.

It was surprisingly found that a semi-solid, a gel, a jelly, a paste, or like compositions of TU when manufactured and filled with unique controlled processing and filling into a hard gelatin capsule confers slower in vitro release, but superior bioperformance and stability with extended shelf life relative to fully solubilized liquid filled TU comprising compositions in a softgel capsule. Moreover, it was also surprisingly found that the TU in the inventive composition (e.g. in semi-solid, gel, jelly, paste, or like dosage forms) when prepared under controlled conditions of this invention resulted in non-crystalline TU in the compositions while meeting or exceeding the typical required performance criteria.

An exemplary commercial embodiment of the therapeutic composition disclosed herein is a capsulized therapeutic composition or “Capsule”. More specifically, a “Capsule” comprises a two piece capsule having a capsule body, a capsule cap that fits over the capsule body, wherein the capsule body and capsule cap are in a closed sealing relationship to each other, and wherein the therapeutic composition is disposed within the closed capsule, and wherein the a band is preferably adhered to the capsule body and capsule cap so as to improve the seal of the closed capsule (see for instance the entry for “Capsule (pharmacy)” on Wikipedia's website). Various variables in the manufacture of such Capsule have been surprisingly and unexpectedly discovered to yield optimal results therefore.

As a background, an exemplary process of manufacturing the “Capsule” is as follows. A quantity of therapeutic composition mixed at a predetermined temperature, and empty capsules (e.g. a capsule body, a capsule cap, and a capsule band) are provided, with the therapeutic composition being adjusted to a predetermined temperature. The therapeutic composition is placed within a (formerly) empty capsule body, a (formerly) empty capsule cap is pressed into a sliding/sealing engagement with the (now filled) capsule body, and the capsule body and capsule cap are pressed together, and a capsule band is placed in sealing/retaining engagement around the capsule body and capsule cap. In commercial (higher volume) manufacturing, the noted process of manufacturing the “Capsule” is performed on relatively high speed filling and banding machines. Exemplary of such machines are for instance a Bosch model GKF 1500 Capsule Filler (an example of which may be seen on the website of AmerEquip International) and a Qualicaps QUALI-SEAL S-100 Two Piece capsule sealing banding machine (an example of which may be seen on the website of LabX). The noted Bosch model GKF 1500 Capsule Filler includes a holding tank or hopper in which the mixed therapeutic composition is placed. As will be appreciated, a full tank of therapeutic composition cannot be processed into “Capsules” instantaneously, and therefore there is a predetermined preferred and maximum hold time and temperature for a given mixture of a composition.

The manufacturing variable of a capsule fill amount is a measurement of the amount of physical volume inside of a capsule which is occupied by the composition, with the remainder (the reciprocal or inverse of the fill volume)—the “head space” volume—being occupied by the atmosphere (e.g. air) present when filling the capsule. Capsule fill amount is typically expressed as a percentage of a capsule body that is occupied by the fill. Thus for instance if a capsule has a fill volume of 80%, it indicates that 80% of the physical volume of the capsule body is occupied by the therapeutic composition. When the “Capsule” is assembled, remaining “head space” would be that portion of the total volume of the assembled capsule not occupied by the fill. Ideally, in order to minimize the total volume/size of a capsule, the fill volume would be maximized or would approach 100% as close as possible. However, it has been shown that an excessive fill volume can result in leakage of the capsule and excessive closed lengths of the capsule (i.e. the capsule body and cap have insufficient overlap when in the closed configuration) due to among other things excessive buildup of pressure within the capsule. A related “real world” constraint is that capsules are typically not commercially available in finite size increments but rather are available in discrete size offerings (e.g. 000, 00EL, 00, 0, 1, 2, 3, 4 and 5) (see for instance the capsule size chart available on LFA Capsule Fillers website).

The manufacturing variable of composition mix temperature, especially for compositions that include solidifying/thickening or viscosity enhancing additives, may be critical in that insufficient temperature may result in a non-homogenous therapeutic composition (which can result in a therapeutic composition having an insufficient amount of a first component/excipient, and an excessive amount of a second component/excipient), and an excessive mix temperature may result in a leaking “Capsule” or a degraded therapeutic composition.

The manufacturing variable of therapeutic composition viscosity may be critical in that too high of a therapeutic composition viscosity (e.g. too thick) may result in poor filing of the “Capsule” and too low of a therapeutic composition viscosity (e.g. too thin) may result in a leaking “Capsule”. It is noted that therapeutic composition viscosity is a function of both the excipients/ingredients of the therapeutic composition and the temperature of the therapeutic composition.

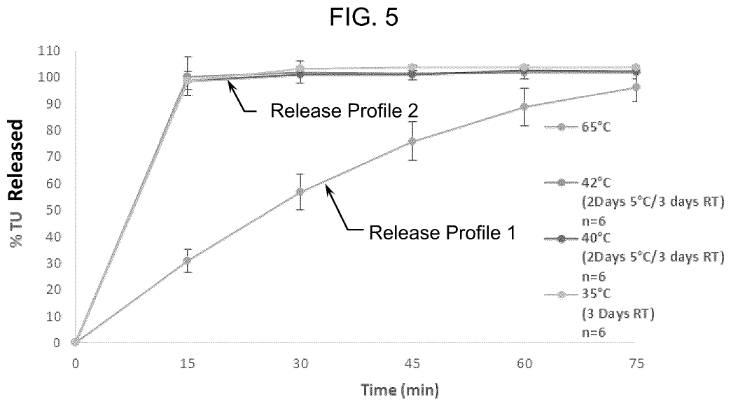

In practice, it was surprisingly and unexpectedly discovered that preferably using a size 00 capsule, a fill volume in the range of 79% to 93%, a mix temperature in the range of 63±5° C. for a duration in the range of 102 to 112 minutes, a fill/hold time and temperature in the range of 60.5±2.5° C. for no more than 119 hours (or alternatively 200 hours, 150 hours, 140 hours, 130 hours, 125 hours, 120 hours, 115 hours, 110 hours, 105 hours, and 100 hours, 95 hours, 90 hours, 85 hours, 80 hours, 75 hours, 72 hours, 70 hours, 68 hours, and 65 hours), and a viscosity in the range of 34 cP to 57 cP (cP=centipoise), resulted a high production efficiency with substantially no leaking or otherwise scrap Capsules and in Capsules that satisfied a required release profile as shown in FIG. 5 (Release Profile 1). The release profile of TU (Release Profile 1 of FIG. 5) has been determined to have release characteristics that include being at about or in between 39% and 59% released at 30 mins and or greater than at or about 80% released at 75 mins. Release profiles for four exemplary mix temperatures of 35° C., 40° C., 42° C., and 65° C. are shown in FIG. 5, where the percent (%) of TU that is released is measured in some instances using dissolution methodology following USP <711>. Conditions for measuring the release profile include an aqueous medium present in a USP Type-II (paddle) apparatus with conditions at 37±0.5° C. and at 100 rpm. In more specific instances, the aqueous medium is about 1 L of DI water having 1% w/w of Triton X-100. The percent (%) of TU that is dissolved is measured every 15 minutes for TU capsules. As shown in FIG. 5, the use of a mix temperature of 65° C. in preparing a capsule changes the resultant release profile for TU, when compared to other exemplary mix temperatures. The mix temperature of 65° C. shows an exemplary ideal release of about 31% after fifteen minutes, 57% after 30 minutes and greater than 80% after 75 mins, while all other exemplary mix temperatures show complete or near complete release within the first 15 minutes. Such release values are slower than those of typical fully solubilized liquid filled compositions (e.g. softgel dosage forms). Various combinations of manufacturing variables outside of the noted ranges resulted in leaking and scrap Capsules in varying amounts and in Capsules that failed to satisfy a required release profile. More specifically, by way of example only, when employing a Bosch model 1500 Capsule Filler machine running at 21-28 spm (Strokes per Minute) and an 5-100 Two Piece capsule sealing banding machine, production rates of 15,000 to 20,000 112.5 mg API Capsules per hour could be achieved with substantially no leaking or otherwise scrap Capsules.

The manufacturing variable of composition pre-fill in a capsule mix or holding temperature may further be critical in that insufficient temperature may result in at least a portion resulting in a crystalline or partially crystalline solid TU comprising composition (which can result in a dosage form having a poor therapeutic release). The manufacturing variable of composition pre-fill in a capsule mix or holding temperature may also be critical to obtaining an adequate oral bioavailability and bioperformance with acceptable or enhanced stability/extended shelf life. Optimal composition pre-fill mix/holding temperature may enable a pharmaceutical dosage form with a semi-solid, a gel, a jelly, a paste, or like composition inside a capsule at room temperature with non-crystalline or amorphous TU which could allow for an acceptable release profile. In one aspect, the capsule and the fill material maintains physical stability (e.g., a non-crystalline material maintains non-crystallinity over an extended shelf life such as >2.5 years).

The pharmaceutical composition with the semi-solid, a gel, a jelly, a paste, or like fill at room temperature, non-crystalline TU may be identified by comparing a DSC (Differential Scanning Calorimetry profile) for crystalline TU, and the pharmaceutical composition with TU and without TU, that is, a placebo form of the composition where the composition differs by the absence of TU when compared to the pharmaceutical composition with TU. Differences in the DSC will show an absence of crystalline or semicrystalline TU signal in the pharmaceutical composition with TU that is observed in the crystalline TU DSC, and additionally no significant differences for any other semicrystalline signal that is observed in the placebo DSC. DSC may additionally be used to characterize oxidative and structural/physical stability of the pharmaceutical composition with TU.

A PHOSITA will appreciate that the determination of an optimal mix temperature or mix time may require a higher temperature, or a longer time when modification to the pharmaceutical composition is performed. For example, if the amount of 40 hydrogenated castor oil is increased (e.g., 20-25% w/w %), a higher temperature, a longer time, or a higher temperature and a longer time may be required to achieve an optimal release profile, similar to the exemplary release profile provided in FIG. 5. By way of other example, if the pharmaceutical composition selected employs an exemplary replacement of a lipophilic carrier (e.g., reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), PEG-20 almond glycerides (Crovol A40), lipophilic polyoxyethylene-polyoxypropylene block co-polymers (Pluronic® L92, L101, L121 etc.); propylene glycol fatty acid esters, such as propylene glycol monolaurate (Lauroglycol FCC), propylene glycol ricinoleate (Propymuls), propylene glycol monooleate (Myverol® P-06), propylene glycol dicaprylate/dicaprate (Captex® 200), and propylene glycol dioctanoate (Captex® 800), propylene glycol mono-caprylate (Capryol® 90); propylene glycol oleate (Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (Arlacel® 186); sterol and sterol derivatives such as cholesterol, sitosterol, phytosterol, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (Arlacel® 20), sorbitan monopalmitate (Span-40), sorbitan monooleate (Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; and mixtures thereof) with another lipophilic carrier (e.g., propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (Arlacel® 20), sorbitan monopalmitate, sorbitan monooleate, sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof) a higher temperature, a longer time, or a higher temperature and a longer time may be required to achieve an optimal release profile, similar to the exemplary release profile provided in FIG. 5. By way of other example, if the pharmaceutical composition selected employs an exemplary replacement of a solidifying agent (e.g., polyethylene glycols; sorbitol; gelatin; stearic acid; cetyl alcohol; cetosterayl alcohol; paraffin wax; polyvinyl alcohol; glyceryl stearates; glyceryl distearate; glyceryl monostearate; glyceryl palmitostearate; glyceryl behenate; waxes; hydrogenated castor oil; hydrogenated vegetable oil; bees wax, microcrystalline wax; sterols; phytosterols; cholesterol and mixtures thereof with another solidifying agent (e.g., polyethylene glycol; gelatin; stearic acid; polyvinyl alcohol; glyceryl stearates; glyceryl distearate; glyceryl monostearate; glyceryl palmitostearate; hydrogenated castor oil; hydrogenated vegetable oil and cholesterol.) a higher temperature, a longer time, or a higher temperature and a longer time may be required to achieve an optimal release profile, similar to the exemplary release profile provided in FIG. 5. By way of other example, if the pharmaceutical composition selected employs an exemplary replacement of a hydrophilic surfactant (e.g., polysorbates, polyoxyethylene hydrogenated vegetable oils, polyoxyethylene vegetable oils; polyoxyethylene sorbitan fatty acid esters; polyoxyethylene-polyoxypropylene block copolymers; polyglycerol fatty acid esters; polyoxyethylene glycerides; polyoxyethylene sterols, derivatives and analogues thereof; reaction mixtures of polyols and at least one member of the group consisting of fatty acids, glycerides, vegetable oils, hydrogenated vegetable oils, fractionated oils and sterols; tocopheryl polyethylene glycol succinates; sugar esters; sugar ethers; sucroglycerides; mixtures thereof; alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers such as poloxamer-108, 188, 217, 238, 288, 338, 407, 124, 182, 183, 212, 331, or 335, or combinations thereof) with another hydrophilic surfactant (e.g., ionic hydrophilic surfactants such as sodium dodecyl sulphate, docusate sodium; bile acid, cholic acid, deoxycholic acid, chenodeoxycholic acid, salts thereof, and mixtures thereof) a higher temperature, a longer time, or a higher temperature and a longer time may be required to achieve an optimal release profile, similar to the exemplary release profile provided in FIG. 5. It is noted that glyceryl monooleate is described as “consists of mono-, di- and triglycerides of mainly linoleic (C_{18:2}) and oleic (C_{18:1}) acids, the diester fraction being predominant”. By way of example only, mix time may be about or greater than 102 minutes, about or greater than 112 minutes, about or greater than 122 minutes, about or greater than 132 minutes, about or greater than 142 minutes, about or greater than 152 minutes, or about or greater than 182 minutes. Mix times of shorter durations may also be possible. Similarly, by way of example only, mix temperature may be about or greater than 58° C., about or greater than 65° C., about or greater than 70° C., about or greater than 75° C., or about or greater than 80° C. Lower mix temperatures may also be possible.

What is claimed is:

1. A method of preparing a pharmaceutical product comprising:
providing a pharmaceutical composition and a capsule, wherein said capsule comprises a capsule body and a capsule cap, and wherein said pharmaceutical composition comprises TU in an amount of at least one of about 112.5±25 mg, 112.5±20 mg, 112.5±15 mg, 112.5±10 mg, 112.5±7.5 mg, and 112.5±5 mg;
inserting a predetermined quantity of said pharmaceutical composition at a predetermined insertion temperature and at a predetermined TU loading strength into said capsule body, said predetermined insertion temperature being in the range of at least one of about 60.5±5° C., about 60.5±3.5° C., about 60.5±2.5° C., about 60.5±2.0° C., about 60.5±1.5° C., and about 60.5±1.0° C., and said predetermined TU loading strength comprising in a w/w % of total composition fill weight, at least one of about 10-20, about 12-18, and about 14-16; and
combining said capsule body with said capsule cap so as to substantially seal said pharmaceutical composition therein, thereby resulting in said prepared pharmaceutical product.

2. The method of claim 1, wherein said pharmaceutical composition further comprises at least one of:
(i) a lipophilic carrier in a w/w % of said pharmaceutical composition amount of at least one of about 40-80, about 45-75, about 50-70, about 55-65, and about 57-63;
(ii) a hydrophilic surfactant in a w/w % of said pharmaceutical composition amount of at least one of about 2-24, about 5-30, about 8-25, about 10-20, about 12-18, and about 14-16; and
(iii) a solidifying agent.

3. The method of claim 1, wherein said pharmaceutical composition comprises a non-liquid pharmaceutical composition.

4. The method of claim 3, wherein said non-liquid pharmaceutical composition comprises at least one of a semi-solid, a gel, a jelly, and a paste.

5. The method of claim 3, wherein said pharmaceutical product, in comparison to a comparable pharmaceutical product having a liquid pharmaceutical composition, has a shelf life that is greater than said comparable pharmaceutical product by more than at least one of 10%, 15%, 25%, 50%, 75%, 100%, 125%, 150%, 200%, and 250%.

6. The method of claim 1, wherein said pharmaceutical product has a shelf life of greater than at least one of 2.5 years, 3.0 years, 4.0 years, and 5.0 years.

7. The method of claim 1, wherein administration of said pharmaceutical product to a subject results in a serum testosterone level average concentration (C_{avg}) of at least one of about 200 ng/dL to about 800 ng/dL, about 300 ng/dL to about 700 ng/dL, about 400 ng/dL to about 600 ng/dL, about 450 ng/dL to about 550 ng/dL, and about 475 ng/dL to about 525 ng/dL.

8. The method of claim 1, wherein said method includes a holding step, said holding step comprising holding said pharmaceutical composition prior to said inserting step for a predetermined holding duration, said predetermined holding duration comprising a duration of less than at least one of 200 hours, 150 hours, 140 hours, 130 hours, 125 hours, 120 hours, 115 hours, 110 hours, 105 hours, and 100 hours, 95 hours, 90 hours, 85 hours, 80 hours, 75 hours, 72 hours, 70 hours, 68 hours, and 65 hours.

9. The method of claim 1, wherein said combining comprises the step of banding, said banding step comprising adhering a band to a joint formed between said capsule body and said capsule cap.

10. The method of claim 1, wherein said method comprises the step of mixing said pharmaceutical composition prior to said inserting.

11. The method of claim 10, wherein said mixing comprises at least one of mixing at a temperature range of about 63±° 5 C, mixing until said pharmaceutical composition is substantially clear, and mixing until said pharmaceutical composition is substantially homogeneous.

12. The method of claim 1, wherein when said capsule comprises a size of 00, said pharmaceutical product comprises a capsule fill volume in the range of about 89% to 93%.

13. The method of claim 1, wherein said lipophilic carrier comprises at least one of glyceryl monolinoleate, an amount of about 50-70 w/w % of said pharmaceutical composition, and glyceryl monolinoleate in an amount of about 50-70 w/w % of said pharmaceutical composition.

14. The method of claim 1, wherein said pharmaceutical product comprises at least one of:
an acceptable release profile,
a scrap rate of less than at least one of 5%, 4%, 3%, 2.75%, 2.5%, 2.25%, and 2.0%,
and a production rate of pharmaceutical products per hour per capsule fill machine of more than at least one of 12,500, 15,000, 17,500, 20,000, 25,000, 30,000, 35,000, 40,000, 45,000, and 50,000.

15. The method of claim 1, wherein when at least one of said predetermined quantity is outside of the range of about 750±25 mg, said predetermined insertion temperature is outside of the range of 60.5±5.0° C., and said predetermined TU loading strength is outside of the range of about 10% to 20%, said pharmaceutical product comprises at least one of an unacceptable release profile, a scrap rate of more than 5%, and a production rate of less than about 12,500 of said pharmaceutical products per hour per capsule fill machine.

16. The method of claim 1, wherein said TU comprises a non-crystalline form TU.

17. A method of preparing a pharmaceutical product comprising:
providing a non-liquid pharmaceutical composition and a capsule, wherein said capsule comprises a capsule body and a capsule cap, and wherein said non-liquid pharmaceutical composition comprises TU in an amount of about 112.5±11.25 mg;
inserting a predetermined quantity of said non-liquid pharmaceutical composition at a predetermined insertion temperature and TU loading strength into said capsule body, said predetermined insertion temperature being in the range of about 60.5±2.5° C., and said predetermined TU loading strength comprising about 12-18 w/w % of total composition fill weight;
combining said capsule body with said capsule cap so as to substantially seal said non-liquid pharmaceutical composition therein, thereby resulting in said prepared pharmaceutical product.

18. The method of claim 17, wherein said pharmaceutical composition further comprises at least one of:
(i) a lipophilic carrier in a w/w % of said pharmaceutical composition amount of about 50-70;
(ii) a hydrophilic surfactant in a w/w % of said pharmaceutical composition amount of about 10-20; and
(iii) a solidifying agent.

19. The method of claim 17, wherein said non-liquid pharmaceutical composition comprises at least one of a semi-solid, a gel, a jelly, and a paste.

20. The method of claim 17, wherein said pharmaceutical product, in comparison to a comparable pharmaceutical product having a liquid pharmaceutical composition, has a shelf life that is greater than said comparable pharmaceutical product by more than at least one of 10%, 15%, 25%, 50%, 75%, 100%, 125%, 150%, 200%, and 250%.

21. The method of claim 17, wherein said pharmaceutical product has a shelf life of greater than at least one of 2.5 years, 3.0 years, 4.0 years, and 5.0 years.

22. The method of claim 17, wherein administration of said pharmaceutical product to a subject results in a serum testosterone level average concentration (C_{avg}) of at least one of about 200 ng/dL to about 800 ng/dL, about 300 ng/dL to about 700 ng/dL, about 400 ng/dL to about 600 ng/dL, about 450 ng/dL to about 550 ng/dL, and about 475 ng/dL to about 525 ng/dL.

23. The method of claim 17, wherein said method includes a holding step, said holding step comprising holding said non-liquid pharmaceutical composition prior to said inserting step for a predetermined holding duration, said predetermined holding duration comprising a duration of less than at least one of 200 hours, 150 hours, 140 hours, 130 hours, 125 hours, 120 hours, 115 hours, 110 hours, 105 hours, and 100 hours, 95 hours, 90 hours, 85 hours, 80 hours, 75 hours, 72 hours, 70 hours, 68 hours, and 65 hours.

24. The method of claim 17, wherein said combining comprises the step of banding, said banding step comprising adhering a band to a joint formed between said capsule body and said capsule cap.

25. The method of claim 17, wherein said method comprises the step of mixing said non-liquid pharmaceutical composition prior to said inserting.

26. The method of claim 25, wherein said mixing comprises at least one of mixing at a temperature range of about 63±° 5 C, mixing until said non-liquid pharmaceutical composition is substantially clear, and mixing until said non-liquid pharmaceutical composition is substantially homogeneous.

27. The method of claim 17, wherein when said capsule comprises a size of 00, said pharmaceutical product comprises a fill volume in the range of about 89% to 93%.

28. The method of claim 17, wherein said lipophilic carrier comprises glyceryl monolinoleate in an amount of about 50-70 w/w % of said pharmaceutical composition.

29. The method of claim 17, wherein said pharmaceutical product comprises at least one of:
an acceptable release profile,
a scrap rate of less than at least one of 5%, 4%, 3%, 2.75%, 2.5%, 2.25%, and 2.0%,
and a production rate of pharmaceutical products per hour per capsule fill machine of more than at least one of 12,500, 15,000, 17,500, 20,000, 25,000, 30,000, 35,000, 40,000, 45,000, and 50,000.

30. The method of claim 17, wherein when at least one of said predetermined quantity is outside of the range of about 750±25 mg, said predetermined insertion temperature is outside of the range of 60.5±5.0° C., and said predetermined TU loading strength is outside of the range of about 12% to 18%, said pharmaceutical product comprises at least one of an unacceptable release profile, a scrap rate of more than 5%, and a production rate of less than about 12,500 of said pharmaceutical products per hour per capsule fill machine.

31. The method of claim 17, wherein said TU comprises a non-crystalline form TU.

32. A method of preparing a pharmaceutical product comprising the ordered steps of:
providing a non-liquid pharmaceutical composition and a capsule, wherein said capsule comprises a capsule body and a capsule cap, and wherein said non-liquid pharmaceutical composition comprises TU in an amount of about 112.5±11.25 mg;
mixing said non-liquid pharmaceutical composition;
holding said non-liquid pharmaceutical composition for no more than 115 hours;
inserting a predetermined quantity of said non-liquid pharmaceutical composition at a predetermined insertion temperature and TU loading strength into said capsule body, said predetermined insertion temperature being in the range of about 60.5±2.0° C., and said predetermined TU loading strength comprising about 14-16 w/w % of total composition fill weight;
combining said capsule body with said capsule cap; and
banding said capsule body with said capsule cap to a joint formed between said capsule body and said capsule cap so as to substantially seal said non-liquid pharmaceutical composition therein, thereby resulting in said prepared pharmaceutical product.

33. The method of claim 32, wherein said pharmaceutical composition further comprises at least one of:
(i) a lipophilic carrier in a w/w % of said pharmaceutical composition amount of about 55-65;
(ii) a hydrophilic surfactant in a w/w % of said pharmaceutical composition amount of about 12-18; and
(iii) a solidifying agent.

34. The method of claim 32, wherein said non-liquid pharmaceutical composition comprises at least one of a semi-solid, a gel, a jelly, and a paste.

35. The method of claim 32, wherein said pharmaceutical product, in comparison to a comparable pharmaceutical product having a liquid pharmaceutical composition, has a shelf life that is greater than said comparable pharmaceutical product by more than at least one of 10%, 15%, 25%, 50%, 75%, 100%, 125%, 150%, 200%, and 250%.

36. The method of claim 32, wherein said pharmaceutical product has a shelf life of greater than at least one of 2.5 years, 3.0 years, 4.0 years, and 5.0 years.

37. The method of claim 32, wherein administration of said pharmaceutical product to a subject results in a serum testosterone level average concentration (C_{avg}) of at least one of about 200 ng/dL to about 800 ng/dL, about 300 ng/dL to about 700 ng/dL, about 400 ng/dL to about 600 ng/dL, about 450 ng/dL to about 550 ng/dL, and about 475 ng/dL to about 525 ng/dL.

38. The method of claim 32, wherein said mixing comprises at least one of mixing at a temperature range of about 63±° 5 C, mixing until said non-liquid pharmaceutical composition is substantially clear, and mixing until said non-liquid pharmaceutical composition is substantially homogeneous.

39. The method of claim 32, wherein when said capsule comprises a size of 00, said pharmaceutical product comprises a fill volume in the range of about 89% to 93%.

40. The method of claim 32, wherein said lipophilic carrier comprises glyceryl monolinoleate in an amount of about 55-65 w/w % of said pharmaceutical composition.

41. The method of claim 32, wherein said pharmaceutical product comprises at least one of:
an acceptable release profile,
a scrap rate of less than at least one of 5%, 4%, 3%, 2.75%, 2.5%, 2.25%, and 2.0%,
and a production rate of pharmaceutical products per hour per capsule fill machine of more than at least one of 12,500, 15,000, 17,500, 20,000, 25,000, 30,000, 35,000, 40,000, 45,000, and 50,000.

42. The method of claim 32, wherein when at least one of said predetermined quantity is outside of the range of about 750±25 mg, said predetermined insertion temperature is outside of the range of 60.5±5.0° C., and said predetermined TU loading strength is outside of the range of about 12% to 18%, said pharmaceutical product comprises at least one of an unacceptable release profile, a scrap rate of more than 5%, and a production rate of less than about 12,500 of said pharmaceutical products per hour per capsule fill machine.

43. The method of claim 32, wherein said TU comprises a non-crystalline form TU.
